(19) United States
(12) Patent Application Publication
(10) Pub. No.: US 2009/0142271 A1
(43) Pub. Date: Jun. 4, 2009

(54) DENDRITIC CELLS

(75) Inventors: Karen Bulloch, Hillsborough, NJ (US); Bruce McEwen, Hillsborough, NJ (US); Ralph Steinman, Westport, CT (US); Ulrike Waltraud Kaunzner, New York, NY (US); Jennifer C. Felger, New York, NY (US); Judit Gal Toth, White Plains, NY (US); Melinda M. Miller, New York, NY (US); Andres C. Gottfried-Blackmore, New York, NY (US)

Correspondence Address: CHOATE, HALL & STEWART LLP, TWO INTERNATIONAL PLACE, BOSTON, MA, 02110, US

(73) Assignee: The Rockefeller University, New York, NY (US)

(21) Appl. No.: 12/211,019

(22) Filed: Sep. 15, 2008

Related U.S. Application Data
(60) Provisional application No. 60/972,397, filed on Sep. 14, 2007.
(60) Provisional application No. 61/052,015, filed on May 9, 2008.

(51) Int. Cl. A61K 49/00; C12N 5/00; C12N 5/02; C12Q 1/68; C12Q 1/02

(52) U.S. Cl. 424/9.1; 435/325; 435/378; 435/6; 435/28

(57) ABSTRACT

The present invention provides systems for isolating, characterizing, and modulating brain dendritic cells. The invention may allow a better understanding of and opportunities to modulate neurodevelopmental processes (such as, for example, neurogenesis) and neurological diseases (such as, for example, Alzheimer's Disease).

### RELATED APPLICATIONS

This application claims the benefit of, and priority to, U.S. Provisional Patent Applications 60/972,397, filed Sep. 14, 2007 and 60/052,015, filed May 9, 2008, the contents of which are hereby incorporated by reference.

### BACKGROUND

Dendritic cells (DCs) of the immune system process antigens and display them to other cells of the immune system. These specialized antigen-presenting cells are involved in initiating immune responses and inducing T cell tolerance to self antigens. Such events are known to occur in the periphery (i.e., in regions outside the central nervous system).

### SUMMARY

DCs have also been found in the brain during central nervous system inflammation and may be involved in the autoimmune diseases in the brain. It was believed that dendritic cells are only recruited into the brain during inflammation and/or disease.

The present invention encompasses the discovery that dendritic cells are present in a normal, non-inflamed, and non diseased brain. The inventors have discovered that these newly discovered dendritic cells, termed “brain dendritic cells” (bDC), can be found in early embryonic stages (for example, as early as embryonic stage E10 in mice). The inventors have also discovered that bDC are present in both embryonic and adult brain in regions associated with neurogenesis. Without wishing to be bound by any particular theory, early presence of bDC in embryonic brains suggests that they may originate and reside there, rather than only infiltrating into the brain during conditions such as disease, inflammation, and/or trauma to the brain. Resident bDC may be involved in certain biological processes such as neurogenesis.

The potential roles for bDC in both normal biology and in disease/injury states suggest that such cells may be useful in diagnostic and therapeutic applications. The present invention provides systems for characterizing, isolating, and/or identifying modulators of such brain dendritic cells that may be useful in developing such diagnostic and therapeutic applications.

In one aspect, the invention provides isolated brain dendritic cells obtained from a mammalian brain that is not inflamed or diseased. In some embodiments, such isolated brain dendritic cells are characterized as having a marker phenotype selected from the group consisting of CD11c+, CD11b+, Iba-1+, CD45+, F4/80+, NeuN−, DCX−, NG2−, GFAP−, and combinations thereof. Isolated brain dendritic cells may have a marker phenotype that is {CD11c+, CD11b+, Iba-1+, CD45+, F4/80+, NeuN−, DCX−, NG2−, and GFAP−}.

In another aspect, the invention provides methods for identifying brain dendritic cells. In certain embodiments, methods of identifying brain dendritic cells comprise steps of providing a transgenic animal that expresses a detectable agent under the control of the CD11c promoter; detecting the agent in brain cells; determining, based on the presence of the agent in a given cell, that the cell is a brain dendritic cell. The detectable agent may be a fluorescent molecule, such as green fluorescent protein or a derivative thereof (such as yellow fluorescent protein). In some embodiments, the method further comprises determining expression of CD11b, Iba-1, CD45, F4/80, or a combination thereof, wherein expression of one or more such markers is an indicator that the cell is a brain dendritic cells.

In certain embodiments, methods for identifying brain dendritic cells in a tissue sample are provided. Such methods generally comprise steps of providing a map that indicates the distribution of brain dendritic cells in the brain of an animal at a given age; providing an image of the tissue sample from a test animal, wherein the tissue sample comprises at least one cell suspected of being a brain dendritic cells; comparing the image of the tissue sample with a map from the atlas, wherein the map indicates distribution of brain dendritic cells at an age comparable to that of the age of the test animal; and identifying, based on the comparison, that the at least one cell is a brain dendritic cell. The map may be part of a series of maps comprising an atlas, wherein maps in the series represent distribution maps of brain dendritic cells at various ages. The tissue sample may have been processed to detect a marker selected from the group consisting of CD11c, CD11b, Iba-1, CD45, F4/80, and combinations thereof. Provided also are methods of isolating brain dendritic cells comprising steps of identifying brain dendritic cells using an atlas as described above and isolating the identified brain dendritic cells from the sample.

In yet another aspect, the invention provides methods of isolating brain dendritic cells. In certain embodiments, such methods comprise steps of providing a transgenic mammal that expresses a detectable agent under the control the CD11c promoter; detecting the agent in brain cells or brain tissue; and isolating cells that express the agent from cells that do not express the agent. In some such embodiments, the detectable agent is a fluorescent molecule, such as, for example, yellow fluorescent protein. The step of isolating may comprise performing fluorescence-activated cell sorting and/or laser capture microdissection. In certain embodiments, methods of isolating generally comprise steps: providing a starting population of cells obtained from brain tissue; and sorting the population of cells into subpopulations based on characteristics of brain dendritic cells, wherein at least one of the subpopulations comprises a substantially higher proportion of brain dendritic cells than that of the starting population.

In another aspect, the invention provides methods of identifying genes that are specifically expressed in brain dendritic cells comprising steps of obtaining RNA from brain dendritic cells, and detecting or identifying one or more genes that are differentially regulated in the sample as compared to a control sample. Brain dendritic cells may be isolated during particular periods of development, and/or during a period during which a particular developmental process is known to take place. For example, isolation may be performed during neurogenesis, gliogenesis, synaptogenesis, embryogenesis, and/or apoptosis. Alternatively or additionally, brain dendritic cells may be isolated from an animal with a condition, such as ischemic injury, excitotoxic injury, autoimmune disorders, and combinations thereof. In another aspect, the invention provides methods of detecting or identifying genes involved in a neurological disease comprising a step of detecting or identifying one or more genes that are differentially regulated in an animal that is a model for a neurodegenerative disease and that contains detectably labeled brain dendritic cells as compared to a control sample, wherein the control sample comprises RNA obtained from cells from an animal that is not a model for the neurological disease.

In some embodiments, the transgenic animal bears a transgene for a detectable agent under the control of the CD11c promoter. The detectable agent may be, for example, a fluorescent molecule such as yellow fluorescent protein. In some embodiments, the neurological disease is Alzheimer's Disease, Parkinson's Disease, Huntington's Disease, multiple sclerosis, or combinations thereof.

In another aspect, the invention provides methods of identifying agents that modulated brain dendritic cells comprising steps of providing a sample that contains brain dendritic cells; contacting the sample with a test agent; determining whether the test agent modulates one or more aspects of brain dendritic cell development, activity, gene expression, and localization; and identifying, based on the determination, that the test agent as a modulator of brain dendritic cells. In some embodiments, the aspect comprises an activity selected from the group consisting of expression of MHC (major histocompatibility complex) molecules, cytokines, cytokine receptors, and combinations thereof. The expression may be induced by a cytokine, such as, for example, interferon gamma. The activity may comprise expression of MHC class II molecules, and/or it may comprise expression of a cytokine such as TNFα, IL-6, nitric oxide, or combinations thereof. In some embodiments, the activity comprises expression of a gene selected from the group consisting of resistin-like alpha, CCL17, CxCL9, CD209a (DC-Sign), H2-Eb1, Spp1 (Osteopontin), Axl, H2-Aa, H2-Al, CxCl2 (MIP-2), Clec7a (Dectin-1), CCR2, Itgax (CD11c), IGF-1, CD36, or a combinations thereof.

These and other aspects of the invention will be appreciated by those of ordinary skill reviewing the present specification.

### BRIEF DESCRIPTION OF THE DRAWING

The patent application or file contains at least one drawing executed in color. Copies of this patent or patent application publication with color drawings will be provided by the Office upon request and payment of the necessary fee.

FIG. 1 depicts photomicrographs showing three morphologies of EYFP+bDC within different brain regions of the healthy developing and adult EYFP−CD11c mouse. (A) Examples of the ovoid morphology in the PN2 brain (arrow). These cells were the most common shape found during development. (B) Photomicrograph of bipolar-like cells in the adult choroid plexus. Note that the more ovoid cell (arrow) has begun to take on a bipolar shape by extending its processes. (C) Asymmetrical, stellate/dendriform EYFP+bDC were evident in the subgranular zone and granular cell layer (gcl) of the adult hippocampal dentate gyrus. Their processes extended throughout the gcl, terminating at the border between the gcl and inner molecular layer (iml) and at the interface between the hilus (hil) and the gcl. (D) Photomicrograph illustrating the stellate morphology exhibited by EYFP+bDC in layer II of the piriform cortex and many other regions throughout the parenchyma of the adult brain. Scale bars=50 μm in (A), (C), and (D) and 25 μm in (B).

FIG. 2 depicts confocal fluorescence photomicrographs demonstrating colocalization of immune cell/microglia marker antibodies with EYFP+bDC in different regions of the brain. EYFP+bDC colocalized with antibodies against (A) Iba-1 in layer II of the piriform cortex, (B) Mac-1 (CD11b) in the subventricular zone (SVZ), and (C) F4/80 in the cerebellum. EYFP+bDC did not colocalize in any region examined with the neuronal marker NeuN, as shown in the interstitial nucleus of the posterior limb of the anterior commissure (IPAC) (D), the postmitotic neuronal marker doublecortin (Dcx), as noted in the hippocampal granular cell layer (gcl) (E), the astrocyte expressing marker GFAP, as noted in the SVZ (F), and the proteoglycan cell marker NG2, in the arcuate nucleus (G). Scale bars=50 μm.

FIG. 3 depicts ultrastructural photomicrographs of EYFP+ bDC in the granule cell layer of the hippocampal dentate gyrus and in the striatum. (A) EYFP− immunopositive (gold particles, arrow) bDC juxtaposed to an unlabeled terminal (ut). (B) Gold particle-labeled EYFP+ bDC showed an abundance of rough endoplasmic reticulum (ER) and mitochondria (M). (C) EYFP+ bDC gold-labeled cell body (double arrow) embedded between granule cells (gc) and juxtaposed to Dcx peroxidase-labeled postmitotic immature neuron processes (single arrow). The EYFP+ bDC had an elongated nucleus with clusters of heterochromatin and scant cytoplasm. The images in A and C were taken from the dentate gyrus; the image in B was taken from the striatum. Scale bars=100 nm in A; 500 nm in B; 2 μm in C.

FIG. 4 shows results from FACS analyses of PN2 CD11c-EYFP brain cells cultured for 2 weeks in media containing GM-CSF (5 ng/ml). EYFP+ bDC responded to the GM-CSF with a marked increase (63%) over control, and the CD11c integrin was expressed in 44% of these cells in a form recognized by the antibody N418. (A) Isotype staining in control cells. (B) CD11c staining in control cells. (C) Isotype staining of GM-CSF controls. (D) CD11c staining in GM-CSF cells (n=2).

FIG. 5 shows relative RNA expression levels of CD11c in EYFP+ bDC as compared to that of EYFP− brain cells. EYFP+ bDC and EYFP− cells were sorted from adult mouse brain parenchyma by FACS. After RNA extraction and first-strand synthesis, mRNA expression was quantified by gene chip and RT-PCR analysis. (A) Data from three separate groups of animals analyzed in three separate gene chip assays revealed a sevenfold increase in Itgax (CD11c) mRNA expression in EYFP+ bDC compared with EYFP− brain cells. (B) These findings were confirmed by RT-PCR on three separate occasions using RNA from a single group of animals. Data are represented as the mean±SE. *P<0.001.

FIG. 6 depicts schematic representations of the distribution EYFP+ bDC. Anterior to posterior coronal diagrams were generated and adapted from Atlas Navigator (2000). Each mark represents approximately two cells. Depicted diagrams represent areas where concentrations of EYFP+ bDC were consistently noted in the young adult male Itgax (CD11c)-EYFP transgenic mouse.

FIG. 7 depicts photomicrographs of EYFP+ bDC detected by immunocytochemistry using anti-GFP antibody (Table 1) and the ABC method. Regions of interest correspond to the diagrams in FIG. 6A-M. (A) EYFP+ bDC were present in the olfactory bulb in the anterior end of the rostral migratory stream within the collapsed ventricles (arrow) and in the olfactory glomerular layer (double arrows). (B) The stellate type of EYFP+ bDC were noted in layer II of the piriform cortex. (C) EYFP+ bDC were embedded in the anterior forceps of the corpus callosum and extended into the subventricular zone. (D) Stellate/dendriform EYFP+ bDC were present in the hippocampal gcl occupying nonoverlapping zones (see also FIG. 1C). (E) Cross-section of the anterior commissure showing bipolar EYFP+ bDC (arrow). (F) Many stellate EYFP+ bDC are noted in the IPAC and ventral palladium (VP), where there is a convergence of sensory nerves. (G) EYFP+ bDC lined the arcuate nucleus. Stellate EYFP+ bDC were consistently evident in the periaqueductal gray (H), in the cerebellum (I), and in the ventral lateral nuclei of the spinal cord (J). The circumventricular organs displayed all three forms of EYFP+ bDC (ovoid, elongated/bipolar-like, and stellate/dendriform) as seen in the subfornical organ (K) and the area postrema (L). Scale bars=200 μm in A, C, and F and 50 μm in B, D, E, and G-L.

FIG. 8 depicts photomicrographs of EYFP+ bDC in mouse embryonic stage E10 brain tissue by staining for GFP antibody (which recognizes EYFP) and visualization with DAB staining. EYFP+ bDC were present in E10 brains including in regions associated with neurogenesis.

FIG. 9 depicts photomicrographs showing EYFP+ bDC stained by the ABC anti-GFP immunocytochemical method in E16 brains. (A) EYFP+ bDC lined the aqueduct and third ventricle (arrow). (B) Ovoid (arrow) and bipolar-like EYFP+ bDC were noted within the developing choroid plexus. (C) Stellate (arrow) EYFP+ bDC were observed within the developing vitreous humor region of the eye. Scale bar=50 μm in A, 25 μm in B, and 100 μm in C.

FIG. 10 depicts photomicrographs of EYFP+ bDC in P0 brain tissue by staining for GFP antibody and visualization using the ABC method. EYFP+ bDC were present in P0 brains including in regions associated with neurogenesis.

FIG. 11 depicts photomicrographs showing EYFP+ bDC stained by the ABC anti-GFP immunocytochemical method in PN2 brains. (A) Fluorescent photomicrograph showing an example of EYFP+ bDC in PN2 cultures. (B) Stained sections revealed EYFP+ bDC with an ovoid (arrow) and stellate/dendriform morphology within the parenchyma of PN2 brains. (C) Bipolar-like (arrow) and stellate EYFP+ bDC were also observed along and near the lateral ventricle of the brain. Scale bars=25 μm in A; 50 μm in B and C.

FIG. 12 depicts photographs showing the difference in the cell distribution and number of bDC in young versus aged animals. Photomicrographs were taken as negatives with a light-table (Northern light, Research incorp./Kaiser, Germany) and show CD11c/EYFP+ cells stained by anti-GFP antibodies and visualized by the ABC method in brains of young (A) versus aged (B) mouse brain. In young brains, CD11c/EYFP+ cells are scattered in distinct regions, like the piriform cortex, dentate gyrus and Hypothalamus, subventricular zone and hypothalamus in the young animal. In aged brains, CD11c/EYFP+ cells show a significant overall increase, and an increase in special regions like the fiber tracts and several cortex regions in the aged animals. 1: Dentate gyrus; 2: Piriform cortex; 3: Median Eminence.

FIG. 13 depicts photomicrographs showing the difference between numbers of bDC in the young versus aged cortex. Tissue sections were stained with anti-GFP antibodies and visualized using the ABC method. (A1 and A2) show sections from prefrontal conrtex and (B1 and B2) show sections from entorhinal/perirhinal/ectorhinal cortex from young (A1 and B1) and aged (A2 and B2) animals. Scale bar=200 μm. M2=second. motor cortex; Cgl=cingulate cortex, area 1; PrL=prelimbic cortex; B1: Ect=ectorhinal cortex; PRh=perirhinal cortex; LEnt=lat. entorhinal cortex.

FIG. 14 shows stereological cell counts of EYFP+ bDC in young and aged cortex. Graphs show the difference in cell number of EYFP+ cells in the (A) prefrontal and (B) entorhinal cortices of young versus aged brains. Brains from aged mice had roughly two and half as many times the number of EYFP+ bDC in both cortical areas as did brains from young mice. **=p<0.001

FIG. 15 illustrates the difference between numbers of bDC in young versus aged corpus callosum, forceps. (A) and (B) depict photomicrographs of tissue sections stained with anti-GFP antibodies and visualized using the ABC method; EYFP+ cells stain positively. Scale bar=200 μm; (C) shows stereological cell counts of EYFP+ bDC in young and aged corpus callosum. Aged corpous callosum had roughly twice as many EYFP+ bDC as did young corpus callosum. *=p<0.01, LV=lateral ventricle.

FIG. 16 illustrates the difference between numbers of bDC in young versus aged cerebellum. (A) and (B) depict photomicrographs of tissue sections stained with anti-GFP antibodies and visualized using the ABC method; EYFP+ cells stain positively. Scale bar=200 μm; (C) shows stereological cell counts of EYFP+ bDC in young and aged cerebellum. Cerebellum from aged mice had roughly twice as many EYFP+ bDC as did cerebellum from young mice. **=p<0.001. Scale bar=200 μm.

FIG. 17 illustrates the difference between numbers of bDC in young versus aged hippocampus and dentate gyrus. (A) and (B) depict photomicrographs of tissue sections stained with anti-GFP antibodies and visualized using the ABC method; EYFP+ cells stain positively. Scale bar=200 μm; (C) shows stereological cell counts of EYFP+ bDC in young and aged cerebellum. Dentate gyrus from aged mice had roughly half as many EYFP+ bDC as did dentate gyrus from young mice. **p<0.001. Scale bar=200 μm.

FIG. 18 shows the difference in numbers of EYFP+ bDC versus microglia in the young versus aged corpus callosum, forceps. (A) and (B) depict photomicrographs showing microglia stained with anti-Iba-1 antibodies and EYFP+ bDC stained with anti-GFP antibodies in the corpus callosum of young versus aged animals (A,B). Scale bar=200 μm; (C) shows a graph showing the stereological difference in the cell number of CD11c/EYFP+ cells in the corpus callosum of young versus aged brains, *=p<0.01; red indicates Iba-1 staining; green indicates EYFP staining.

FIG. 19 depicts photomicrographs comparing the morphological activation of EYFP+ bDC with EGFP+ microglia from the cfms (CSF-1R)-EGFP mouse in the hippocampal formation 48 hours following kainic acid (KA)-induced seizures. (A) Photomicrograph of the CA3 region of the hippocampus in a KA-treated cfms (CSF-1R)-EGFP transgenic mouse, showing EGFP+ microglia in the region of neuronal damage. Activated morphology was displayed by these cells, as indicated by their shortened processes and hypertrophied cell bodies. (B) Large, activated stellate/dendriform EYFP+ bDC clustered around the CA3 region where neuronal cell loss occurs. (C) Photomicrograph of a cfms (CSF-1R) EGFP brain showing EGFP+ microglia outside of the KA-damaged zones (hippocampal dentate gyrus) maintained an alert to steady-state morphology. (D) Photomicrograph showing a dramatic relocation of EYFP+ bDC to the subgranular zone and in hilus of the dentate gyrus. Scale bars=100 μm.

FIG. 20 depicts photomicrographs showing EYFP+ bDC in mice following a medial cerebral artery occlusion (MCAO) model of stroke. By 6 hours after reperfusion, EYFP+ bDC can be detected in the cortex in the ischemic hemisphere. By 3 days post-reperfusion, bDC are present in the core region and prominent in the penumbra region.

FIG. 21 depicts photomicrographs of tissues stained with immune markers (Iba-1, CD45, Cd11c, and MHC class II) expressed by bDC in the penumbra and core regions 3 day post-MCAO and reperfusion. bDC showed different marker staining patterns in the penumbra and core regions.

FIG. 22 shows relative expression levels of genes involved in secretion/extracellular space. SPP1 (osteopontin) and Apoe (apolipoprotein E) were each overexpressed by approximately 19.5 and 5.4 fold in EYFP+ bDC as compared to EYFP− microglia. Lpl (liporpotein lipoase) was overexpressed by approximately 6.8 fold in EYFP+ bDC. Retnla-1 (resistin like alpha) is overexpressed by over 142 fold in EYFP+ bDC.

FIG. 23 shows relative expression levels of cell surface receptor genes in EYFP+ bDC as compared to EYFP− microglia. CD72 (C-type lectin (Lyb-2), Axl (a receptor tyrosine kinase), CD11c (Integrin αX, Itgax), CD209a (DC-SIGN), Alcom (CD166)m and Ccr2 (chemokine (C—C motif) receptor 2) were each overexpressed several fold in EYFP+ bDC.

FIG. 24 shows relative expression levels of genes encoding products involved in antigen presentation in EYFP+ bDC as compared to EYFP− microglia. H2-EB1, H2-Abl, and H2-Aa each encode MHC class II (major histocompatibility 2, class II antigen) molecules and were each overexpressed by at least 15 fold in EYFP+ bDC.

FIG. 25 shows relative expression levels of cytokine genes in EYFP+ bDC as compared to EYFP− microglia. Cxcl9, CCL-17, and Cxcl2 were all overexpressed in EYFP+ bDC by over 43, over 36 and over 13 fold respectively.

FIG. 26 shows levels of LPS-stimulated production of cytokines by EYFP+ bDC and EYFP− microglia. Production of TNFα, IL-6, and nitric oxide (NO) are shown.

FIG. 27 depict Western blot analyses of MHC class II and IL-1β expression in LPS-stimulated EYFP+ bDC and EYFP− microglia.

FIG. 28 shows experimental results demonstrating that bDC are induced by IFNγ to express MHC class II. (A) Western blot showing MHC class II expression in a mixed culture of both microglia and bDC following IFNγ induction. Loaded in the first lane is a control sample from spleen; loaded in the second and third lanes are samples from an unstimulated mixture of brain microglia/bDC; loaded in the last three lanes are a mixture of microglia and bDC cells stimulated with IFN-gamma (B-E) Immunostaining experiments show colocalization of MHC II staining with EYFP staining.

FIG. 29 depicts photomicrographs of brain tissue after intracerebral injection of INFγ. Tissue sections were stained with anti-GFP antibodies and processed with the ABC method to visualize EYFP+ bDC. INFγ is a potent inducer of bDC in vivo.

FIG. 30 depicts photomicrographs showing morphological differences between bDC in the basal ganglia of (A) a EYFP+ animal and (B) an irradiated wildtype animal restored with bEYFP+ bone marrow.

FIG. 31 depicts photomicrographs from bone marrow chimera experiments performed to determine the origin of INFγ-activated bDC. In brain tissue from EYFP+ hosts transplanted with WT bone marrow (left panel), activated bDC are visible as EYFP+. The right panel shows photomicrographs of brain tissue sections from WT hosts transplanted with EYFP+ bone marrow.

### ABBREVIATIONS AND DEFINITIONS

### Abbreviations

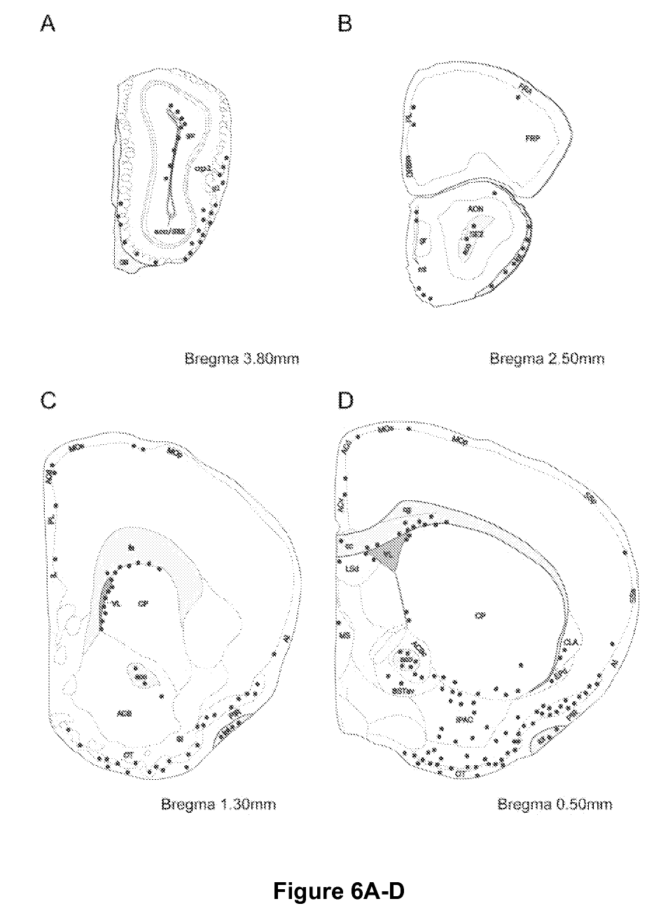

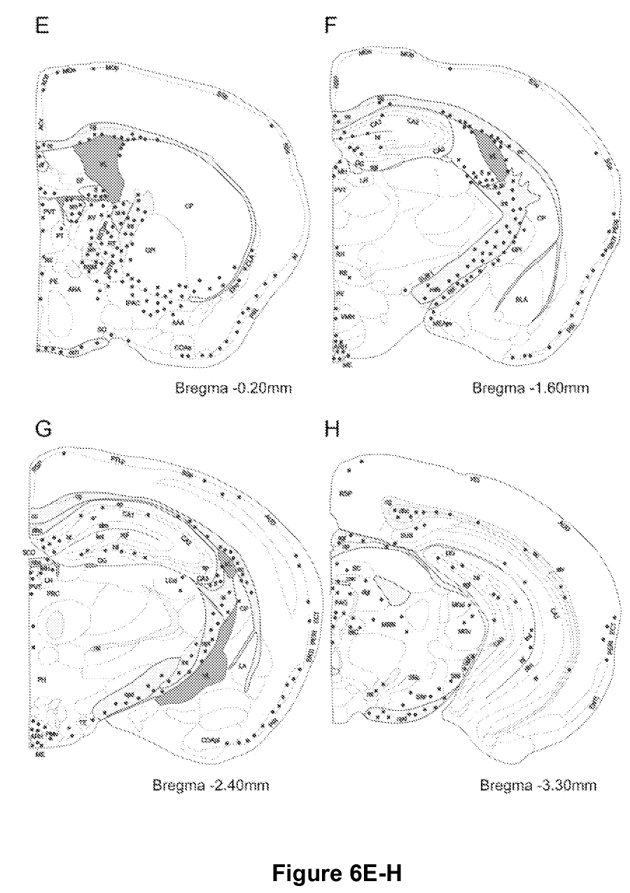

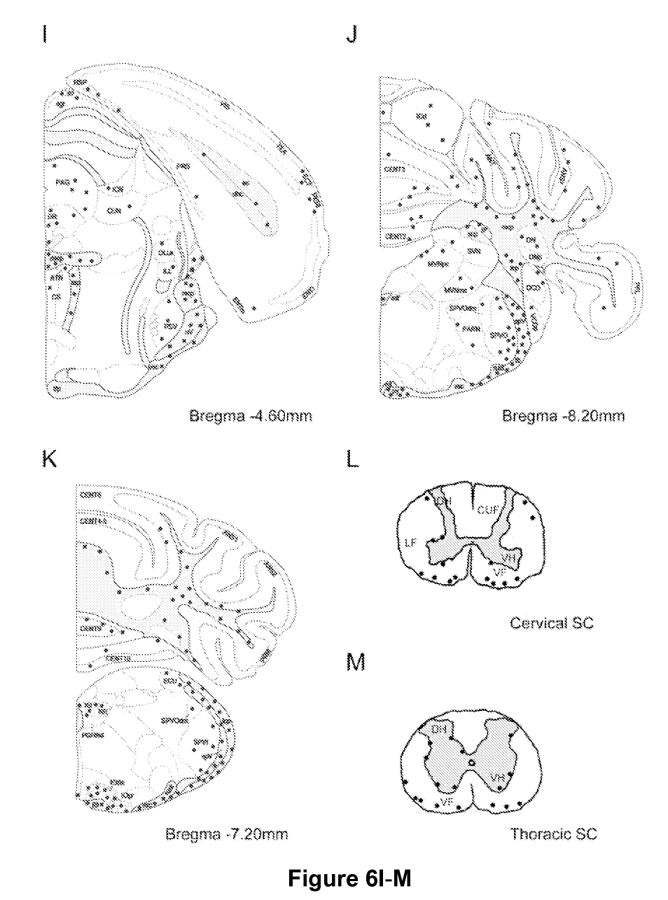

In FIG. 6, certain abbreviations are employed that are described as below.

AAA: anterior amygdaloid area

ACB: nucleus accumbens

ACBc: nucleus accumbens, core

ACd: anterior cingulate cortex, dorsal part

aco: anterior commissure, olfactory limb

Acv: anterior cingulate cortex, ventral part

AHA: anterior hypothalamic area

Al: agranular insula cortex

alv: alveus

ANS1: ansiform lobe of the cerebellum, crus 1

ANS2: ansiform lobe of the cerebellum, crus 2

ANT: anterior lobe of the cerebellum

AON: anterior olfactory nucleus

ARH: arcuate nucleus of the hypothalamus

ATN: anterior tegmental nucleus

AUD: auditory cortex

AV: anteroventral nucleus of the thalamus

BLA: basolateral nucleus of the amygdala

BSM: bed nucleus of the stria medullaris

BSTad: bed nucleus of the stria terminalis, anterodorsal part

BSTif: bed nucleus of the stria terminalis, interfascicular part

BSTpm: bed nucleus of the stria terminalis, posteromedial part

CA1: CA1 field of the Ammon's horn

CA2: CA2 field of the Ammon's horn

CA3: CA3 field of the Ammon's horn

cc: corpus callosum

CENT10: central lobe of the cerebellum, lobule 10 (=nodulus)

CENT2: central lobe of the cerebellum, lobule 2

CENT3: central lobe of the cerebellum, lobule 3

CENT4: central lobe of the cerebellum, lobule 4 (=culmen)

CENT4-5: central lobe of the cerebellum, lobules 4-5 (=culmen)

CENT6: central lobe of the cerebellum, lobule 6 (=declive)

CENT9: central lobe of the cerebellum, lobule 9 (=uvula)

cg: cingulum bundle

CLA: claustrum

COAa: cortical nucleus of the amygdala, anterior part

COApl: cortical nucleus of the amygdala, posterolateral part

CP: caudate-putamen

cpd: cerebral peduncle

csc: commissure of the superior colliculus

CSI: central superior nucleus of the raphe

CUF: cuneate fasciculus, spinal cord

CUN: cuneiform nucleus

DCO: dorsal cochlear nucleus

df: dorsal fornix

DG: dentate gyrus

dgr: deep gray layer of the superior colliculus

DH: dorsal horn of the spinal cord

dhc: dorsal hippocampal commissure

DLL: dorsal nucleus of the lateral lemniscus

DN: dentate nucleus

DNp: dentate nucleus, parvicellular part

DR: dorsal nucleus of the raphe

dscp: decussation of the superior cerebellar peduncle

ec: external capsule

ECT: ectorhinal cortex

ECU: external cuneate nucleus

ENTI: entorhinal cortex, lateral part

ENTm: entorhinal cortex, medial part

EPd: endopiriform nucleus, dorsal part

fa: corpus callosum, anterior forceps

fi: fimbria

FRA: frontal association area

FRP: frontal pole

gcl: granule cell layer, hippocampus

gl: glomerular layer, olfactory bulb

GPI: globus pallidus, lateral part

gr: granule cell layer, olfactory bulb

GU: gustatory cortex

hbc: habenular commissure

hf: hippocampal fissure

hil: hilus, dentate gyrus

ICd: inferior colliculus, dorsal nucleus

ICN: intercollicular nucleus

icp: inferior cerebellar peduncle

IL infralimbic area

ILL: intermediate nucleus of the lateral lemniscus

iml: intramolecular layer, dentate gyrus

INC: interstitial nucleus of Cajal

int: internal capsule

IOda: inferior olivary complex, dorsal accessory nucleus

IOpr: inferior olivary complex, principal olive

IP: nucleus interpositus

IPAC: interstitial nucleus of the posterior limb of the anterior commissure

ipl: internal plexiform layer

LA: lateral nucleus of the amygdala

LF: lateral funiculus, spinal cord

LGd: lateral geniculate nucleus, dorsal part

LH: lateral habenula

lot: lateral olfactory tract

LSd: lateral septum nucleus, dorsal part

mcp: middle cerebellar peduncle

ME: median eminence

MEApv: medial nucleus of the amygdala, posteroventral part

mfb: medial forebrain bundle

MGd: medial geniculate nucleus, dorsal part

MGv: medial geniculate nucleus, ventral part

MH: medial habenula

ml medial lemniscus

mlf medial longitudinal fasciculus

MOp: primary motor cortex

MOs: secondary motor cortex

MRN: mesencephalic reticular nucleus

MS: medial septal nucleus

MVNmc: medial vestibular nucleus, magnocellular part

MVNpc: medial vestibular nucleus, parvicellular part

NR: nucleus of Roller

nV: trigeminal nerve

och: optic chiasm

ON: olfactory nerve (nI)

op: optic layer of the superior colliculus

opl: outer plexiform layer

opt: optic tract

ORBm: orbital cortex, medial part

OT: olfactory tubercle

PAG: periaqueductal gray matter

PARN: parvicellular reticular nucleus

PE: periventricular nucleus of the hypothalamus

PERI: perirhinal cortex

PFL: paraflocculus

PGRNd: paragigantocellular reticular nucleus, dorsal part

PH: posterior hypothalamic area

PIR: piriform cortex

PL: prelimbic area

PM: premammillary nucleus, ventral

PRC: precommissural nucleus

PRM: paramedian lobule of the cerebellum

PRS: presubiculum

PSV: principal sensory nucleus of the trigeminal nerve

PT: paratenial nucleus of the thalamus

PTLp: parietal association cortex, posterior area

PVT: nucleus of the thalamus

py: pyramidal tract

RE: reuniens nucleus of the thalamus

RH: rhomboid nucleus of the thalamus

RSP: retrosplenial cortex

RT: reticular nucleus of the thalamus

rust: rubrospinal tract

SC: superior colliculus

SCO: subcommissural organ

scp: superior cerebellar peduncle

sgl: subgranular layer, dentate gyrus

SEZ: subependymal zone

SF: septofimbrial nucleus

sgr: superficial gray layer of the superior colliculus

slm: stratum lacunosum moleculare, hippocampus

sm stria medullaris

SNc: substantia nigra, compact part

SNl: substantia nigra, lateral part

SNr: substantia nigra, reticular part

SO: supraoptic nucleus

sp: stratum pyramidale, hippocampus

spV: spinal tract of the trigeminal nerve

SPVI: spinal nucleus of the trigeminal nerve, interpolar part

SPVOdm: spinal nucelus of the trigeminal nerve, oral dorsomedial part

sr: stratum radiatum, hippocampus

SSp: primary somatosensory cortex

SSs: secondary somatosensory cortex

st: stria terminalis

SUB: subiculum

SUBI: subincertal nucleus

SVN: superior vestibular nucleus

tb: trapezoid body

TE: terete nucleus of the hypothalamus

TEA: temporal association cortex

tfp: transverse fibers of the pons

tsp: tectospinal tract

VCOp: ventral cochlear nucleus, posterior part

VF: ventral funiculus, spinal cord

VH: ventral horn of the spinal cord

VIS: visual cortex

VL: lateral ventricle

VMHc: ventromedial nucleus of the hypothalamus

vsc: ventral spinocerebellar tract

XII: hypoglossal nucleus

zo: zonal layer of the superior colliculus

### DEFINITIONS

Throughout the specification, several terms are employed that are defined in the following paragraphs.

As used herein, the terms “about” and “approximately,” in reference to a number, is used herein to include numbers that fall within a range of 20%, 10%, 5%, or 1% in either direction (greater than or less than) the number unless otherwise stated or otherwise evident from the context (except where such number would exceed 100% of a possible value).

As used herein, the term “complementary” refers to nucleic acid sequences that base-pair according to the standard Watson-Crick complementary rules, or that are capable of hybridizing to a particular nucleic acid segment under relatively stringent conditions. Nucleic acid polymers are optionally complementary across only portions of their entire sequences.

As used herein, the term “dendritic cell” (abbreviated as “DC”) refers to specialized antigen presenting cells that are involved in initiating immune responses and maintaining tolerance of the immune system to self antigens. Dendritic cells may be found in both lymphoid and non-lymphoid organs and are generally thought to arise from lymphoid or myeloid lineages, though this may not always be the case. The term “peripheral dendritic cell” refers to dendritic cells that are found in the periphery, that is, outside the central nervous system. The term “brain dendritic cell” (abbreviated as “bDC”) refers to a newly characterized class of dendritic cells that are found in the brain, irrespective of its origin in the body or lineage. For example, a dendritic cell that has originated in the periphery but is found in the brain is, unless otherwise noted, a “brain dendritic cell.” Brain dendritic cells may be classified as “resident bDC” (having been present in the brain prior to an incident of interest, such as, for example injury to the brain) or “peripheral bDC” (having infiltrated into the brain from the periphery during or after an incident of interest).

As used herein, the terms “fluorophore”, “fluorescent moiety”, “fluorescent label”, “fluorescent dye” and “fluorescent labeling moiety” are used herein interchangeably. They refer to a molecule which, in solution and upon excitation with light of appropriate wavelength, emits light back. Numerous fluorescent dyes of a wide variety of structures and characteristics are suitable for use in the practice of this invention. Similarly, methods and materials are known for fluorescently labeling nucleic acids (see, for example, R. P. Haugland, “Molecular Probes: Handbook of Fluorescent Probes and Research Chemicals 1992-1994”, 5^{th} Ed., 1994, Molecular Probes, Inc.). In choosing a fluorophore, it is preferred that the fluorescent molecule absorbs light and emits fluorescence with high efficiency (i.e., high molar absorption coefficient and fluorescence quantum yield, respectively) and is photostable (i.e., it does not undergo significant degradation upon light excitation within the time necessary to perform the analysis).

As used herein, the term “gene” refers to a discrete nucleic acid sequence responsible for a discrete cellular product and/or performing one or more intracellular or extracellular functions. In some embodiments, the term “gene” refers to a nucleic acid that includes a portion encoding a protein and optionally encompasses regulatory sequences, such as promoters, enhancers, terminators, and the like, which are involved in the regulation of expression of the protein encoded by the gene of interest. Such gene and regulatory sequences may be derived from the same natural source, or may be heterologous to one another. In some embodiments, a gene does not encode proteins but rather provide templates for transcription of functional RNA molecules such as tRNAs, rRNAs, etc. Alternatively or additionally, in some embodiments, a gene may define a genomic location for a particular event/function, such as the binding of proteins and/or nucleic acids.

As used herein, the term “gene expression” refers to the conversion of the information, contained in a gene, into a gene product. A gene product can be the direct transcriptional product of a gene (e.g., mRNA, tRNA, rRNA, antisense RNA, ribozyme structural RNA or any other type of RNA), or the product of subsequent downstream processing events (e.g., splicing, RNA processing, translation). In some embodiments, a gene product is a protein produced by translation of an mRNA. In some embodiments, gene products are RNAs that are modified by processes such as capping, polyadenylation, methylation, and editing, proteins post-translationally modified, and proteins modified by, for example, methylation, acetylation, phosphorylation, ubiquitination, ADP-ribosylation, myristilation, and glycosylation.

As used herein, the term “isolated” when applied to cells or a population of cells, refers to cells which (a) by virtue of their origin or manipulation, are separated from at least some of the tissues and/or other cells with which they are commonly associated in their natural state; and/or (b) have been manipulated by the hand of man.

As used herein, the terms “labeled”, “labeled with a detectable agent” and “labeled with a detectable moiety” are used interchangeably. They can be used to specify that a cell from a sample can be visualized and/or distinguished from cells that are not labeled. The label may arise from a gene product or molecule present on and/or in the cell. For example, a particular cell type may be “labeled” by a fluorescent protein (such as, for example, yellow fluorescent protein or green fluorescent protein) that is present only in a subset of cell types or in that particular cell type. As another example, a cell may produce a molecule that can be detected by methods such as, for example, staining with antibodies, cleavage of certain substrates that result in detectable cleavage products, etc. Alternatively or additionally, molecules such as antibodies, nucleic acids, etc. may themselves by “labeled” such that they may be visualized by methods known in the art. The detectable agent or moiety may be selected such that it generates a signal whose intensity can be measured. The detectable agent or moiety may be selected such that it generates a localized signal, thereby allowing spatial resolution of the signal from other labels within a tissue, collection of cells, etc. In hybridization array experiments, it may be desirable for the detectable agent or moiety to generate a localized signal to allow spatial resolution between spots on the array. Methods for labeling molecules such as antibodies and nucleic acids are known in the art. Labeled molecules can be prepared by incorporation of or conjugation to a label, that is directly or indirectly detectable by spectroscopic, photochemical, biochemical, immunochemical, electrical, optical, or chemical means. Suitable detectable agents include, but are not limited to: various ligands, radionuclides, fluorescent dyes, chemiluminescent agents, microparticles, enzymes, calorimetric labels, magnetic labels, and haptens. Detectable moieties can also be biological molecules such as molecular beacons and aptamer beacons.

As used herein, the term “marker” refers to its meaning as understood in the art. The term can refer to an indicator that provides information as to the type of cell that expresses or does not express the marker. The term “marker” can also refer to a molecule that is the subject of an assay or measurement the result of which provides information about a cell type. For example, an elevated expression level of a particular gene can be an indicator that a cell is of a certain type, a certain lineage, and of a certain activation or resting state. The expression level of the gene, an elevated expression level of the gene, and the gene expression product (such as a protein) itself, can all be referred to as “markers.”

As used herein, the term “messenger RNA” or “mRNA” refers a form of RNA that serves as a template for protein biosynthesis. In many embodiments, the amount of a particular mRNA (i.e., having a particular sequence, and originating from a particular same gene) reflects the extent to which the gene encoding the mRNA has been “expressed.”

As used herein, the term “neurodevelopmental disease” (used interchangeable with “neurodevelopmental disorder”) refers to a subset of neurological disorders that involve impairment of growth and/or development of the nervous system. Neurodevelopmental diseases include, but are not limited to, autism, Rett syndrome, schizophrenia, Prader-Willi syndrome, Angelman syndrome, Williams syndrome, Down syndrome, Fragile X syndrome, etc.

As used herein, the term “neurodegenerative disease” refers to a subset of neurological diseases (see below) in which degeneration of tissues of the nervous system occurs. For example, cells of the brain and/or spinal cord may be lost. Examples of neurodegenerative diseases include, but are not limited to, Alexander's disease, Alper's disease, Alzheimer's disease, Amyotrophic lateral sclerosis, Ataxia telangiectasia, Batten disease (also known as Spielmeyer-Vogt-Sjogren-Batten disease), Bovine spongiform encephalopathy (BSE), Canavan disease, Cockayne syndrome, Corticobasal degeneration, Creutzfeldt-Jakob disease, Frontotemporal lobar degeneration, Huntington's disease, HIV-associated dementia, Kennedy's disease, Krabbe's disease, Lewy body dementia, Machado-Joseph disease (Spinocerebellar ataxia type 3), Multiple System Atrophy, multiple sclerosis, Neuroborreliosis, Parkinson's disease, Pelizaeus-Merzbacher Disease, Pick's disease, primary lateral sclerosis, prion diseases, progressive Supranuclear Palsy, Refsum's disease, Sandhoffs disease, Schilder's disease, subacute combined degeneration of spinal cord secondary to Pernicious Anaemia, Spielmeyer-Vogt-Sjogren-Batten disease (also known as Batten disease), spinocerebellar ataxia, spinal muscular atrophy, Steele-Richardson-Olszewski disease, Tabes dorsalis, etc.

As used herein, the term “neurological disease” may be used interchangeably with “encephalopathy” and refers to a disorder of the nervous system. Such disorders include, but are not limited to and Alzheimer's Disease, Parkinson's Disease, Huntington's Disease, amyotrophic lateral sclerosis (Lou Gehrig's disease), multiple sclerosis, kuru, schizophrenia, etc. Disorders characterized by certain symptoms such as ataxia, dyskinesia, chorea, athetosis, nerve compression, paralysis, atopgnosia, atopognosis, etc. are also generally considered neurological disease.

As used herein, the terms “nucleic acid” and “nucleic acid molecule” are used herein interchangeably. They refer to a deoxyribonucleotide or ribonucleotide polymer in either single- or double-stranded form, and unless otherwise stated, encompass known analogs of natural nucleotides that can function in a similar manner as naturally occurring nucleotides. The terms encompass nucleic acid-like structures with synthetic backbones, as well as amplification products.

As used herein, the term “oligonucleotide” refers to usually short strings of DNA or RNA to be used as hybridizing probes or nucleic acid molecule array elements. These short stretches of sequence are often chemically synthesized. The size of the oligonucleotide depends on the function or use of the oligonucleotides. When used in microarrays for hybridization, oligonucleotides can comprise natural nucleic acid molecules or synthesized nucleic acid molecules and comprise between 5 and 150 nucleotides, preferably between about 15 and about 100 nucleotides, more preferably between 15 and 30 nucleotides and most preferably, between 18 and 25 nucleotides complementary to mRNA.

As used herein, the term “RNA transcript” refers to the product resulting from transcription of a DNA sequence. When the RNA transcript is the original, unmodified product of a RNA polymerase catalyzed transcription, it is referred to as the primary transcript. An RNA transcript that has been processed (e.g., spliced, etc.) will differ in sequence from the primary transcript; a fully processed transcript is referred to as a “mature” RNA. The term “transcription” refers to the process of copying a DNA sequence of a gene into an RNA product, generally conducted by a DNA-directed RNA polymerase using the DNA as a template. A processed RNA transcript that is translated into protein is often called a messenger RNA (mRNA).

As used herein, the terms “subject” and “individual” are used herein interchangeably. They refer to a human or another animal (e.g., mouse, rat, rabbit, dog, cat, cattle, swine, sheep, horse, or primate) that can be afflicted with or is susceptible to a disease, disorder, condition, or complication but may or may not have the disease or disorder. In many embodiments, the subject is a human being.

### DETAILED DESCRIPTION OF CERTAIN EMBODIMENTS OF THE INVENTION

As mentioned above, the present invention provides systems for isolating and/or characterizing brain dendritic cells (bDC), as well as methods of identifying genes and/or modulators involved in neurological disease and/or neurodevelopmental processes.

### I. Isolated Brain Dendritic Cells

In one aspect, the invention provides isolated brain dendritic cells from a mammal that is not suffering from an inflammatory disease or condition. The brain of the mammal may be in a so-called “steady state” condition, i.e., not suffering from an abnormal condition, such as disease, injury, etc. In some embodiments, the mammal is a human. In some embodiments, the mammal may be an animal that is used in biomedical research (such as disease models, genetically modified and/or modifiable organisms, animals in which antibodies are raised or biological materials are extracted, etc.). Examples of such mammals include, but are not limited to, non-human primates (such as rhesus macaques, pig-tailed macaques, cynomolgus macaques, owl monkeys, capuchin monkeys, squirrel monkeys, tamarins, common marmosets, chimpanzees, etc.), rodents (such as rats, mice, hamsters, etc.), guinea pigs, ferrets, dogs, rabbits, horses, donkeys, goats, cattle, pigs, sheep, etc.

Isolated bDC are typically characterized by detectable expression of certain markers and low or undetectable expression of other markers. For example, isolated bDC may have detectable expression of CD11c (also known as Integrin αX and Itgax), CD11b (also known as Integrin α_{M}, Mac-1α), Iba-1 (ionized calcium binding adapter molecule 1; also known as AIF1, allograft inflammatory factor 1), CD45 (also known as common leukocyte common antigen (LCA), Ly-5, T200 and/or B220 in mice), F4/80 in mice (or EMR1 in humans), or a combination of such markers. Isolated bDC typically have no or low levels of detectable expression of certain neuronal and glial markers such as NeuN (neuronal specific nuclear protein), DCX (also known as doublecortin), NG2 (a proteoglycan) and GFAP (glial fibrillary acidic protein). Isolated bDC may have a marker phenotype selected from the group consisting of CD11c+, CD11b+, Iba-1+, CD45+, F4/80+, NeuN−, DCX−, NG2−, GFAP−, and combinations thereof. In some embodiments, isolated bDC have a marker phenotype of {CD11c+, CD11b+, Iba-1+, CD45+, F4/80+, NeuN−, DCX−, NG2−, and GFAP−}.

By “detectable expression” it is meant that a gene product (such as protein), gene message (such as RNA), or both is detectable. It is understood that a gene message may sometimes be detectable though the corresponding gene product might not be detectable by certain methods (for example, immunostaining, the sensitivity of which may often depend on characteristics of available antibodies). In such circumstances, the marker is still considered to be “expressed.”

Those of ordinary skill in the art will understand that the names of some or all of the above markers may refer to a particular antigen, protein, or other marker in a particular species, and that the names may differ in other species. For example, F4/80 refers to a mouse antigen, and the human homologue of F4/80 is known as EMR1. Alternatively or additionally, more than one name may exist for a particular marker even within the same species. Such alternative designations and/or embodiments are included in the invention.

Isolated bDC may be cultured (for example, in growth medium with or without other types of cells), frozen, and/or stored. Also, isolated bDC grown in culture may give arise to progeny cells that may include additional bDC.

bDC that have been isolated and cultured, frozen, and/or stored or that have arisen from isolated bDC are encompassed within the term “isolated brain dendritic cells,” even if they should lose certain characteristics of bDC that are freshly isolated from mammalian tissue. Those of ordinary skill in the art will recognize that certain characteristics of cells (such as morphology, marker expression, etc.) may change in culture or during a process such as freezing or storing, yet still be considered for many purposes the same cell type as they were when they were freshly isolated.

Isolated brain dendritic cells may be obtained, for example, by inventive methods of isolating discussed below.

### II. Identifying Brain Dendritic Cells

In some aspects, the invention provides methods of identifying brain dendritic cells. In some embodiments, such methods generally comprises steps of: providing a transgenic animal that expresses a detectable agent under the control of the CD11c (also known as Integrin αX, or Itgax) promoter; detecting the agent in brain cells; and determining, based on the presence of the agent in a given cell, that the cell is a brain dendritic cell. The agent may be detected on the surface of a given cell as well as or instead of inside the cell. Detecting may be of whole brain, of brain tissue, of brain tissue that has been processed, of a population of brain cells (such as a single cell suspension), etc. Such embodiments are included in this invention.

Any of a wide variety of detectable agents can be used in the practice of the present invention. Suitable detectable agents include, but are not limited to: various ligands, radionuclides (such as, for example, ³²P, ³⁵S, ³H, ¹⁴C, ¹²⁵I, ¹³¹I and the like); fluorescent dyes (for specific exemplary fluorescent dyes, see below); chemiluminescent agents (such as, for example, acridinium esters, stabilized dioxetanes and the like); microparticles (such as, for example, quantum dots, nanocrystals, phosphors and the like); enzymes (such as, for example, those used in an ELISA, i.e., horseradish peroxidase, beta-galactosidase, luciferase, alkaline phosphatase); colorimetric labels (such as, for example, dyes, colloidal gold and the like); magnetic labels (such as, for example, Dynabeads™); and biotin, dioxigenin or other haptens and proteins for which antisera or monoclonal antibodies are available.

For example, a fluorophore-containing molecule such as a fluorescent protein may be used as a detectable agent. A variety of known fluorophores can be employed in the practice of the invention. (See, for example, The Handbook: Fluorescent Probes and Labeling Technologies, 10th edition (2005), Invitrogen, Carlsbad, Calif., the entire contents of which are incorporated herein by reference.) Fluorophores that may be used in fluorescent tags include fluorescein, rhodamine, phycobiliproteins, cyanine, coumarin, pyrene, green fluorescent protein, BODIPY®, and their derivatives. Both naturally occurring and synthetic derivatives of fluorophores can be used. Examples of fluorescein derivatives include fluorescein isothiocyanate (FITC), Oregon Green, Tokyo Green, seminapthofluorescein (SNAFL), and carboxynaphthofluorescein. Examples of rhodamine derivatives include rhodamine B, rhodamine 6G, rhodamine 123, tetramethyl rhodamine derivatives TRITC and TAMRA, sulforhodamine 101 (and its sulfonyl chloride form Texas Red), and Rhodamine Red. Phycobiliproteins include phycoerythrin, phycocyanin, allophycocyanin, phycoerythrocyanin, and peridinin chlorophyll protein (PerCP). Types of phycoerythrins include R-phycoerythrin, B-phycoerythrin, and Y-phycoerythrin. Examples of cyanine dyes and their derivatives include Cy2 (cyanine), Cy3 (indocarbocyanine), Cy3.5, Cy5 (indodicarbocyanine), Cy5.5, Cy7, BCy7, and DBCy7. Examples of green fluorescent protein derivatives include enhanced green fluorescent protein (EGFP), blue fluorescent protein (BFP), enhanced blue fluorescent protein (EBFP), cyan fluorescent protein (CFP), yellow fluorescent protein (YFP), and enhanced yellow fluorescent protein (EYFP). BODIPY® dyes (Invitrogen) are named either for the common fluorophore for which they can substitute or for their absorption/emission wavelengths. BODIPY® dyes include BODIPY FL, BODIPY R6G, BODIPY TMR, BODIPY TR, BODIPY 581/591, BODIPY 630/650, and BODIPY 650/665.

Alexa Fluor® dyes (Invitrogen) are also suitable for use in accordance with inventive methods and compounds. Alexa Fluor® dyes are named for the emission wavelengths and include Alexa Fluor 350, Alex Fluor 405, Alexa Fluor 430, Alexa Fluor 488, Alex Fluor 500, Alexa Fluor 514, Alexa Fluor 532, Alexa Fluor 546, Alexa Fluor 555, Alexa Fluor 568, Alexa Fluor 594, Alexa Fluor 610, Alexa Fluor 633, Alexa Fluor 647, Alexa Fluor 660, Alexa Fluor 680, Alexa Fluor 700, and Alexa Fluor 750.

In some embodiments of the invention, the detectable agent is a fluorescent protein such as green fluorescent protein, yellow fluorescent protein, or enhanced derivatives of such proteins such as EGFP or EYFP, which bear minor mutations that result in enhanced fluorescence.

In some embodiments, the detectable agent is an enzyme. For examples, enzymes that enable a reaction whose product is detectable are suitable for use as detectable agents. Examples of suitable enzymes include, but are not limited to, those used in an ELISA, e.g., horseradish peroxidase, beta-galactosidase, luciferase, and alkaline phosphatase. Other examples include beta-glucuronidase, beta-D-glucosidase, urease, glucose oxidase plus peroxide and alkaline phosphatase.

Methods of detecting agents such as those discussed above are well known in the art. For example, fluorescent detectable agents may be detected using lasers that excite fluorophores coupled with detectors that collect emitted light. Fluorescence detectors may be combined with microscopes to allow viewing of labeled cells, and/or labeled cells may be detected in a machine such as a fluorescence activated cell sorter, which can detect and provide information about single cells. As another example, enzymatic agents may be detected using substrates (which may be naturally present in the cell, and/or may be added externally). For a discussion of commonly used enzyme/substrate combinations, see the below discussion on visualization of immunostained cells.

Methods of generating transgenic animals bearing a particular transgene under the control of a promoter are known in the art. Some transgenic lines suitable for use in accordance with the practice of the invention may be available from laboratories and/or animal repositories. In some embodiments, the transgenic animal is a CD11c-EYFP transgenic mouse.

In some embodiments, methods of identifying further comprise determining expression of a marker such as CD11b, Iba-1, CD45, F4/80 (EMR1 in humans), or combinations thereof. Detectable expression of such markers is an indicator that the cell is a brain dendritic cell. In some embodiments, methods of identifying further comprise determining expression of a marker such as NeuN, NG2 proteoglycan, GFAP, or combinations thereof. Detectable expression of NeuN, NG2 proteoglycan, GFAP, or combinations thereof is an indicator that the cell is not a brain dendritic cell.

In some embodiments, methods of identifying further comprise a step of determining expression of one or more additional markers expressed by the cells. In some such embodiments, methods further comprise determining that one or more of the additional markers is not expressed by other brain cells.

Various methods to determine expression of a given marker are known in the art. Determining expression of a marker can comprise determining whether a protein is present in and/or on a cell. Antibodies that recognize the protein marker may be used. For example, tissues can be immunostained using labeled antibodies or antibodies labeled indirectly (for example, through secondary antibodies).

Immunostained cells can be visualized by a variety of methods known in the art. For example, the ABC method is commonly used and employs biotinylated secondary antibodies that recognize a primary antibody (that recognizes a given marker protein). In the ABC method, an avidin/biotinylated enzyme complex is then added; such a complex binds to the biotinylated secondary antibody. An enzyme substrate is then added, and a detectable enzyme product is produced. The enzyme product may precipitate, allowing visualization of cells that express the marker. Examples of enzymes commonly used in the ABC method include, but are not limited to, alkaline phosphatase (substrates of which include 5-bromo-4-chloro-3-indolyl phosphate and tetrazolium salts such as nitroblue tetrazolium, DuoLoX Chemiluminescent/Fluorescent Substrate (VECTOR labs), p-Nitrophenylphosphate, etc.), glucose oxidase (substrates of which include glucose and tetrazolium salts), peroxidase (substrates of which include 4-chloro-1-napthol, 2,2′-azino-bis(3-ethylbenzthiazoline-6-sulfonic acid, 3-amino-9-ethylcarbazole, 3,3′-diaminobenzidine, 3,3′,5′,5′-tetramethylbenzidine, DuoLuX Chemiluminescent/Fluorescent Substrate for Peroxidase (VECTOR labs), ImmPACT DAB Substrate (VECTOR labs), VECTOR NovaRED Substrate, VECTOR SG Substrate, VECTOR VIP Substrate, etc.).

As another example, antibodies are labeled (directly or indirectly) with a fluorescent label and cells that are stained with such antibodies can be visualized with a fluorescent detector with a microscope. In some embodiments, cells are disrupted from their tissues and optionally processed into a single cell suspension; determining expression of markers in such embodiments may be accomplished using a method such as fluorescence-activated cell sorting.

Determining expression of a marker can comprise determining whether message (such as RNA) of a marker gene is being produced in the cell. For example, in situ RNA hybridization methods may be used to detect RNA message of a marker in tissue sections.

In some embodiments, provided are methods of identifying brain dendritic cells in a brain tissue sample. Such methods generally comprise steps of providing a map that indicates the distribution of brain dendritic cells in the brain of an animal at a given age; providing an image of the brain tissue sample from a test animal, wherein the tissue sample comprises at least one cell suspected of being a brain dendritic cell; comparing the image of the brain tissue sample with a map from the atlas, wherein the map indicates distribution of brain dendritic cells at an age comparable to that of the age of the test animal; and identifying, based on the comparison, that the at least one cell is a brain dendritic cell.

The map may be part of a series of maps comprising an atlas, wherein maps in the series represent distribution maps of brain dendritic cells at various ages. The atlas may comprise distribution maps that indicate the known or expected distribution of bDC at a given age. Ages that may be represented in the atlas may include embryonic, postnatal, and/or adult ages. At least a subset of such ages may represent developmental landmarks for the animal, such as when gastrulation occurs, when neurogenesis occurs, when gliogenesis occurs, when synaptogenesis occurs, when the animal is born, when the animal's eyes open, etc. Alternatively or additionally, the ages represented in the atlas may include ages from regular intervals of embryonic and postnatal development.

The image of the brain tissue sample may be obtained, for example, using a microscope. Images may be compared directly from a microscope to distribution maps in the atlas. In some embodiments, images are processed by computer software or an image processing algorithm before being compared against distribution maps.

Steps of comparing and identifying images and distribution maps may be carried out manually and/or automatically using computer software and/or algorithms.

Brain tissue samples may be processed to detect markers of bDC (such as, for example, CD11c, CD11b, Iba-1, CD45, F4/80, and combinations thereof). For example, brain tissue samples may be stained with antibodies for such markers. Images of such processed/stained tissue samples may then be compared against one or more distribution maps in the atlas.

In some embodiments, the brain tissue sample is a tissue section. Such sections may be thin enough such that staining with reagents such as antibodies can permeate the tissue. Section thickness typically vary between approximately 1 μm and approximately 60 μm, inclusive of endpoints.

### III. Isolating Brain Dendritic Cells

In some aspects, the invention provides methods of isolating brain dendritic cells. In certain embodiments, such methods often comprise steps of providing a transgenic mammal that expresses a detectable agent under the control the CD11c promoter; detecting the agent in brain cells or brain tissue; and isolating cells that express the agent from cells that do not express the agent.

An agent may be detected on the surface of a given cell as well as or instead of inside the cell. Detecting may be of whole brain, of brain tissue, of brain tissue that has been processed, of a population of brain cells (such as a single cell suspension), etc. Such embodiments are included in this invention.

A detectable agent may be an agent as mentioned above for methods of identifying bDC.

In certain embodiments, methods of isolating generally comprise identifying brain dendritic cells in a sample using a map as described above and isolating the identified brain dendritic cells from the sample.

A step of isolating identified brain dendritic cells may comprise performing techniques known in the art such as laser capture microdissection, etc. In laser capture microdissection, cells distinguished by morphology, histological stains, and/or labeling by fluorescent antibodies may be isolated using a laser.

In some embodiments, cells are sorted after optionally being processed into single cell suspensions. Methods for sorting include fluorescence activated cell sorting, which may allow sorting of bDC from other cells. Other methods for sorting include use of surfaces (such as on plastic plates, dishes, wells, etc.), particles (such as on beads, microspheres, etc.), columns, etc. to which antibodies or other molecules that bind to certain bDC specific markers are bound. “Negative” sorting methods may also be used in which antibodies or other molecules that recognize markers of non bDC are used to extract non bDC and remove them from a population of cells comprising bDC.

In some embodiments, such as those involving fluorescence activated cell sorting, the step of identifying and the step of isolating are performed simultaneously.

In certain embodiments, methods of isolating generally comprise steps of providing a starting population of cells obtained from brain tissue and sorting the population of cells into subpopulations based on characteristics of brain dendritic cells, wherein at least one of the subpopulations comprises a substantially higher proportion of brain dendritic cells than that of the starting population. Characteristics of brain dendritic cells that can be used as a basis of sorting include morphology (such as, for example, those discussed in Example 1 below), marker phenotype (such as those discussed above), migration in a density gradient, etc. Combinations of characteristics may be used to sort bDCs. Sorting may be accomplished using any of a variety of methods known in the art, such as those discussed above.

### IV. Gene Expression of Brain Dendritic Cells

In certain aspects, the invention provides methods of identifying genes that are differentially expressed in brain dendritic cells. Such methods comprise steps of obtaining RNA from isolated brain dendritic cells; and detecting or identifying one or more genes that are differentially regulated in the sample as compared to a control sample. In some embodiments, the brain dendritic cells are first identified and/or isolated.

### A. Identifying and Isolating

Steps of identifying and isolating brain dendritic cells may be carried out by methods as discussed above. To identify genes that are differentially expressed in brain dendritic cells and may be involved in a particular function or process, it may be desirable to identify and isolate brain dendritic cells from an animal at a particular age, stage of development, condition, etc. In some embodiments, brain dendritic cells are isolated from an animal during a particular period of development. In some embodiments, brain dendritic cells are isolated from an animal during a period during which a particular developmental process is known to take place. The developmental process may be, for example, neurogenesis, gliogenesis, synaptogenesis, embryogenesis, apoptosis, etc. In some embodiments, the brain dendritic cells are isolated from an animal with one or more conditions such as ischemic injury (for example, due to stroke), excitotoxic injury (for example, due to seizure), autoimmune disorder, etc.

### B. Obtaining RNA

RNA may be obtained from brain dendritic cells (e.g., isolated brain dendritic cells), for example, by any suitable method of RNA isolation or extraction. In some embodiments, RNA is obtained from brain dendritic cells immediately after isolation of brain dendritic cells. In some embodiments, RNA is obtained from brain dendritic cells after one or more processes such as culturing, freezing, storing, etc.

Though the following description refers to processing and/or obtaining RNA after isolation of brain dendritic cells, in some embodiments, certain procedures (such as culturing, freezing, storing, etc.) are performed before the step of isolating brain dendritic cells.

In some embodiments, before isolation or extraction of RNA, isolated brain dendritic cells are stored for a certain period of time under suitable storage conditions. In some embodiments, suitable storage conditions comprise temperatures ranging between about 10° C. to about −220° C., inclusive. In some embodiments, samples are stored at about 4° C., at about −10° C., at about −20° C., at about −70° C., or at about −80° C. In some embodiments, samples are stored for more than about twenty-four hours. In some embodiments, before freezing, an RNase inhibitor, which prevents degradation of RNA by RNases (i.e., ribonucleases), is added to the sample of brain dendritic cells. In some embodiments, the RNase inhibitor is added within two hours of, within one hour of, within thirty minutes of, within ten minutes of, within five minutes of, within two minutes of, or immediately after isolating the cells. In some embodiments, before RNA extraction, the frozen cells are thawed at 37° C. and mixed with a vortex.

In some embodiments, cells are frozen (e.g., flash-frozen in liquid nitrogen and dry ice), stored, and thawed; then RNAse inhibitor is added after thawing. In some such embodiments, the RNase inhibitor is added within two hours, within one hour, within thirty minutes, within ten minutes, within five minutes, or within two minutes, of thawing.

The most commonly used RNase inhibitor is a natural protein derived from human placenta that specifically (and reversibly) binds RNases (P. Blackburn et al., J. Biol. Chem. 1977, 252: 5904-5910). RNase inhibitors are commercially available, for example, from Ambion (Austin, Tex.; as SUPERase-In™), Promega, Inc. (Madison, Wis.; as rRNasin® Ribonuclease Inhibitor) and Applied Biosystems (Framingham, Mass.). In general, precautions for preventing RNases contaminations in RNA samples, which are well known in the art and include the use of gloves, of certified RNase-free reagents and ware, of specifically treated water and of low temperatures, as well as routine decontamination and the like, are used in the practice of the methods of the invention.

Isolating RNA may include treating the cells such that RNA present in the remaining cells is extracted and made available for analysis. Any suitable isolation method that results in extracted RNA may be used in the practice of the invention. It may be desirable to minimize artifacts from manipulation processes. Therefore, the number of extraction and modification steps is in some embodiments kept as low as possible.

Methods of RNA extraction are well known in the art (see, for example, J. Sambrook et al., “Molecular Cloning: A Laboratory Manual”, 1989, 2^{nd} Ed., Cold Spring Harbour Laboratory Press: New York). Most methods of RNA isolation from tissues or cells are based on the disruption of the tissue or cells in the presence of protein denaturants to quickly and effectively inactivate RNases. Generally, RNA isolation reagents comprise, among other components, guanidinium thiocyanate and/or beta-mercaptoethanol, which are known to act as RNase inhibitors (J. M. Chirgwin et al., Biochem. 1979, 18: 5294-5299). Isolated total RNA is then further purified from the protein contaminants and concentrated by selective ethanol precipitations, phenol/chloroform extractions followed by isopropanol precipitation (see, for example, P. Chomczynski and N. Sacchi, Anal. Biochem. 1987, 162: 156-159) or cesium chloride, lithium chloride or cesium trifluoroacetate gradient centrifugations (see, for example, V. Glisin et al., Biochem. 1974, 13: 2633-2637; D. B. Stem and J. Newton, Meth. Enzymol. 1986, 118: 488).

In certain methods of the invention, for example those wherein brain dendritic cell RNA is subjected to a gene-expression analysis, it may be desirable to isolate mRNA from total RNA in order to allow the detection of even low level messages (B. Alberts et al., “Molecular Biology of the Cell”, 1994 (3^{rd} Ed.), Garland Publishing, Inc.: New York, N.Y.).

Purification of mRNA from total RNA typically relies on the poly(A) tail present on most mature eukaryotic mRNA species. Several variations of isolation methods have been developed based on the same principle. In a first approach, a solution of total RNA is passed through a column containing oligo(dT) or d(U) attached to a solid cellulose matrix in the presence of high concentrations of salts to allow the annealing of the poly(A) tail to the oligo(dT) or d(U). The column is then washed with a lower salt buffer to remove and release the poly(A) mRNAs. In a second approach, a biotinylated oligo(dT) primer is added to the solution of total RNA and used to hybridize to the 3′ poly(A) region of the mRNAs. The hybridization products are captured and washed at high stringency using streptavidin coupled to paramagnetic particles and a magnetic separation stand. The mRNA is eluted from the solid phase by the simple addition of ribonuclease-free deionized water. Other approaches do not require the prior isolation of total RNA. For example, uniform, superparamagnetic, polystyrene beads with oligo(dT) sequences covalently bound to the surface may be used to isolate mRNA directly by specific base pairing between the poly(A) residues of mRNA and the oligo(dT) sequences on the beads. Furthermore, the oligo(dT) sequence on the beads may also be used as a primer for the reverse transcriptase to subsequently synthesize the first strand of cDNA. Alternatively or additionally, new methods or improvements of existing methods for total RNA or mRNA isolation, preparation and purification may be devised by one skilled in the art and used in the practice of the methods of the invention.

### C. Detecting or Identifying Differentially Expressed Genes in Brain Dendritic Cells

i. Amplification of Extracted RNA

In certain embodiments, RNA is amplified before being analyzed. In some embodiments, before analysis, the RNA is converted, by reverse-transcriptase, into complementary DNA (cDNA), which, optionally, may, in turn, be converted into complementary RNA (cRNA) by transcription.

Amplification methods are well known in the art (see, for example, A. R. Kimmel and S. L. Berger, Methods Enzymol. 1987, 152: 307-316; J. Sambrook et al., “Molecular Cloning: A Laboratory Manual”, 1989, 2^{nd} Ed., Cold Spring Harbour Laboratory Press: New York; “Short Protocols in Molecular Biology”, F. M. Ausubel (Ed.), 2002, 5^{th} Ed., John Wiley & Sons; U.S. Pat. Nos. 4,683,195; 4,683,202 and 4,800,159). Standard nucleic acid amplification methods include: polymerase chain reaction (or PCR, see, for example, “PCR Protocols: A Guide to Methods and Applications”, M. A. Innis (Ed.), Academic Press: New York, 1990; and “PCR Strategies”, M. A. Innis (Ed.), Academic Press: New York, 1995); and ligase chain reaction (or LCR, see, for example, U. Landegren et al., Science, 1988, 241: 1077-1080; and D. L. Barringer et al., Gene, 1990, 89: 117-122).

Methods for transcribing RNA into cDNA are also well known in the art. Reverse transcription reactions may be carried out using non-specific primers, such as an anchored oligo-dT primer, or random sequence primers, or using a target-specific primer complementary to the RNA for each genetic probe being monitored, or using thermostable DNA polymerases (such as avian myeloblastosis virus reverse transcriptase or Moloney murine leukemia virus reverse transcriptase). Other methods include transcription-based amplification system (TAS) (see, for example, D. Y. Kwoh et al., Proc. Natl. Acad. Sci. 1989, 86: 1173-1177), isothermal transcription-based systems such as Self-Sustained Sequence Replication (3SR) (see, for example, J. C. Guatelli et al., Proc. Natl. Acad. Sci. 1990, 87: 1874-1878), and Q-beta replicase amplification (see, for example, J. H. Smith et al., J. Clin. Microbiol. 1997, 35: 1477-1491; and J. L. Burg et al., Mol. Cell. Probes, 1996, 10: 257-271).

The cDNA products resulting from these reverse transcriptase methods may serve as templates for multiple rounds of transcription by the appropriate RNA polymerase (for example, by nucleic acid sequence based amplification or NASBA, see, for example, T. Kievits et al., J. Virol. Methods, 1991, 35: 273-286; and A. E. Greijer et al., J. Virol. Methods, 2001, 96: 133-147). Transcription of the cDNA template rapidly amplifies the signal from the original target mRNA.

These methods as well as others (either known or newly devised by one skilled in the art) may be used in the practice of the invention.

Amplification can also be used to quantify the amount of extracted RNA (see, for example, U.S. Pat. No. 6,294,338). Alternatively or additionally, amplification using appropriate oligonucleotide primers can be used to label RNA prior to analysis (see below). Suitable oligonucleotide amplification primers can easily be selected and designed by one skilled in the art.

ii. Labeling of RNA

In certain embodiments, RNA (for example, after amplification, or after conversion to cDNA or to cRNA) is labeled with a detectable agent or moiety before being analyzed. The role of a detectable agent is to facilitate detection of RNA or to allow visualization of hybridized nucleic acid fragments (e.g., nucleic acid fragments bound to genetic probes). In some embodiments, the detectable agent is selected such that it generates a signal which can be measured and whose intensity is related to the amount of labeled nucleic acids present in the sample being analyzed. In array-based analysis methods, the detectable agent is also in some embodiments selected such that it generates a localized signal, thereby allowing spatial resolution of the signal from each spot on the array.

Association between the nucleic acid molecule and detectable agent can be covalent or non-covalent. Labeled nucleic acid fragments can be prepared by incorporation of or conjugation to a detectable moiety. Labels can be attached directly to the nucleic acid fragment or indirectly through a linker. Linkers or spacer arms of various lengths are known in the art and are commercially available, and can be selected to reduce steric hindrance, or to confer other useful or desired properties to the resulting labeled molecules (see, for example, E. S. Mansfield et al., Mol. Cell. Probes, 1995, 9: 145-156).

Methods for labeling nucleic acid molecules are well-known in the art. For a review of labeling protocols, label detection techniques and recent developments in the field, see, for example, L. J. Kricka, Ann. Clin. Biochem. 2002, 39: 114-129; R. P. van Gijlswijk et al., Expert Rev. Mol. Diagn. 2001, 1: 81-91; and S. Joos et al., J. Biotechnol. 1994, 35: 135-153. Standard nucleic acid labeling methods include: incorporation of radioactive agents, direct attachment of fluorescent dyes (see, for example, L. M. Smith et al., Nucl. Acids Res. 1985, 13: 2399-2412) or of enzymes (see, for example, B. A. Connoly and P. Rider, Nucl. Acids. Res. 1985, 13: 4485-4502); chemical modifications of nucleic acid fragments making them detectable immunochemically or by other affinity reactions (see, for example, T. R. Broker et al., Nucl. Acids Res. 1978, 5: 363-384; E. A. Bayer et al., Methods of Biochem. Analysis, 1980, 26: 1-45; R. Langer et al., Proc. Natl. Acad. Sci. USA, 1981, 78: 6633-6637; R. W. Richardson et al., Nucl. Acids Res. 1983, 11: 6167-6184; D. J. Brigati et al., Virol. 1983, 126: 32-50; P. Tchen et al., Proc. Natl. Acad. Sci. USA, 1984, 81: 3466-3470; J. E. Landegent et al., Exp. Cell Res. 1984, 15: 61-72; and A. H. Hopman et al., Exp. Cell Res. 1987, 169: 357-368); and enzyme-mediated labeling methods, such as random priming, nick translation, PCR and tailing with terminal transferase (for a review on enzymatic labeling, see, for example, J. Temsamani and S. Agrawal, Mol. Biotechnol. 1996, 5: 223-232). More recently developed nucleic acid labeling systems include, but are not limited to: ULS (Universal Linkage System; see, for example, R. J. Heetebrij et al., Cytogenet. Cell. Genet. 1999, 87: 47-52), photoreactive azido derivatives (see, for example, C. Neves et al., Bioconjugate Chem. 2000, 11: 51-55), and alkylating agents (see, for example, M. G. Sebestyen et al., Nat. Biotechnol. 1998, 16: 568-576).

As discussed above in the section on methods of identifying brain dendritic cells, any of a wide variety of detectable agents can be used in the practice of the present invention.

In certain embodiments, RNA (after amplification, or conversion to cDNA or to cRNA) is fluorescently labeled. Numerous known fluorescent labeling moieties of a wide variety of chemical structures and physical characteristics are suitable for use in the practice of this invention. Suitable fluorescent dyes include, but are not limited to: Cy-3™, Cy-5™, Texas red, FITC, phycoerythrin, rhodamine, fluorescein, fluorescein isothiocyanine, carbocyanine, merocyanine, styryl dye, oxonol dye, BODIPY dye (i.e., boron dipyrromethene difluoride fluorophore, see, for example, C. S. Chen et al., J. Org. Chem. 2000, 65: 2900-2906; C. S. Chen et al., J. Biochem. Biophys. Methods, 2000, 42: 137-151; U.S. Pat. Nos. 4,774,339; 5,187,288; 5,227,487; 5,248,782; 5,614,386; 5,994,063; and 6,060,324), and equivalents, analogues, derivatives or combinations of these molecules. Similarly, methods and materials are known for linking or incorporating fluorescent dyes to biomolecules such as nucleic acids (see, for example, R. P. Haugland, “Molecular Probes: Handbook of Fluorescent Probes and Research Chemicals 1992-1994”, 5^{th} Ed., 1994, Molecular Probes, Inc.). Fluorescent labeling dyes as well as labeling kits are commercially available from, for example, Amersham Biosciences, Inc. (Piscataway, N.J.), Molecular Probes, Inc. (Eugene, Oreg.), and New England Biolabs, Inc. (Beverly, Mass.).

Favorable properties of fluorescent labeling agents to be used in the practice of the invention include high molar absorption coefficient, high fluorescence quantum yield, and photostability. Some labeling fluorophores exhibit absorption and emission wavelengths in the visible (i.e., between 400 and 750 nm) rather than in the ultraviolet range of the spectrum (i.e., lower than 400 nm).

In other embodiments, RNA (for example, after amplification or conversion to cDNA or cRNA) is made detectable through one of the many variations of the biotin-avidin system, which are well known in the art. Biotin RNA labeling kits are commercially available, for example, from Roche Applied Science (Indianapolis, Ind.) Perkin Elmer (Boston, Mass.), and NuGEN (San Carlos, Calif.).

Detectable moieties can also be biological molecules such as molecular beacons and aptamer beacons. Molecular beacons are nucleic acid molecules carrying a fluorophore and a non-fluorescent quencher on their 5′ and 3′ ends. In the absence of a complementary nucleic acid strand, the molecular beacon adopts a stem-loop (or hairpin) conformation, in which the fluorophore and quencher are in close proximity to each other, causing the fluorescence of the fluorophore to be efficiently quenched by FRET (i.e., fluorescence resonance energy transfer). Binding of a complementary sequence to the molecular beacon results in the opening of the stem-loop structure, which increases the physical distance between the fluorophore and quencher thus reducing the FRET efficiency and allowing emission of a fluorescence signal. The use of molecular beacons as detectable moieties is well-known in the art (see, for example, D. L. Sokol et al., Proc. Natl. Acad. Sci. USA, 1998, 95: 11538-11543; and U.S. Pat. Nos. 6,277,581 and 6,235,504). Aptamer beacons are similar to molecular beacons except that they can adopt two or more conformations (see, for example, O. K. Kaboev et al., Nucleic Acids Res. 2000, 28: E94; R. Yamamoto et al., Genes Cells, 2000, 5: 389-396; N. Hamaguchi et al., Anal. Biochem. 2001, 294: 126-131; S. K. Poddar and C. T. Le, Mol. Cell. Probes, 2001, 15: 161-167).

A “tail” of normal or modified nucleotides may also be added to nucleic acid fragments for detectability purposes. A second hybridization with nucleic acid complementary to the tail and containing a detectable label (such as, for example, a fluorophore, an enzyme or bases that have been radioactively labeled) allows visualization of the nucleic acid fragments bound to the array (see, for example, system commercially available from Enzo Biochem Inc., New York, N.Y.).

The selection of a particular nucleic acid labeling technique will depend on the situation and will be governed by several factors, such as the ease and cost of the labeling method, the quality of sample labeling desired, the effects of the detectable moiety on the hybridization reaction (e.g., on the rate and/or efficiency of the hybridization process), the nature of the detection system to be used, the nature and intensity of the signal generated by the detectable label, and the like.

iii. Analysis of RNA

According to the present invention, RNA can be analyzed to obtain information regarding the RNA. In certain embodiments, analyzing the RNA comprises determining the quantity, concentration or sequence composition of RNA.

RNA from brain dendritic cells may be analyzed by any of a variety of methods. Methods of analysis of RNA are well-known in the art (see, for example, J. Sambrook et al., “Molecular Cloning: A Laboratory Manual”, 1989, 2nd Ed., Cold Spring Harbour Laboratory Press: New York, N.Y.; and “Short Protocols in Molecular Biology”, 2002, F. M. Ausubel (Ed.), 5th Ed., John Wiley & Sons).

For example, the quantity and concentration of RNA extracted from brain dendritic cells may be evaluated by UV spectroscopy, wherein the absorbance of a diluted RNA sample is measured at 260 and 280 nm (W. W. Wilfinger et al., Biotechniques, 1997, 22: 474-481). Quantitative measurements may also be carried out using certain fluorescent dyes, such as, for example, RiboGreen® (commercially available from Molecular Probes, Eugene, Oreg.), which exhibit a large fluorescence enhancement when bound to nucleic acids. RNA labeled with these fluorescent dyes can be detected using standard fluorometers, fluorescence microplate reader or filter fluorometers. Another method for analyzing quantity and quality of RNA samples is through use of a BioAnalyzer (commercially available from Agilent Technologies, Foster City, Calif.), which separates charged biological molecules (such as nucleic acids) using microfluidic technologies and then a laser to excite intercalating fluorescent dyes.

RNA from brain dendritic cells may also be analyzed through sequencing. For example, RNase T1, which cleaves single-stranded RNA specifically at the 3′-side of guanosine residues in a two-step mechanism, may be used to digest denatured RNA. Partial digestion of 3′ or 5′ labeled RNA with this enzyme thus generates a ladder of G residues. The cleavage can be monitored by radioactive (M. Ikehara et al., Proc. Natl. Acad. Sci. USA, 1986, 83: 4695-4699) and photometric (H. P. Grunert et al., Protein Eng. 1993, 6: 739-744) detection systems, by zymogram assay (J. Bravo et al., Anal. Biochem. 1994, 219: 82-86), agar diffusion test (R. Quaas et al., Nucl. Acids Res. 1989, 17: 3318), lanthan assay (C. B. Anfinsen et al., J. Biol. Chem. 1954, 207: 201-210) or methylene blue test (T. Greiner-Stoeffele et al., Anal. Biochem. 1996, 240: 24-28) or by fluorescence correlation spectroscopy (K. Korn et al., Biol. Chem. 2000, 381: 259-263).

Other methods for analyzing RNA include northern blots, wherein the components of the RNA sample being analyzed are resolved by size prior to detection thereby allowing identification of more than one species simultaneously, and slot/dot blots, wherein unresolved mixtures are used.

In certain embodiments, analyzing RNA from brain dendritic cells comprises submitting the extracted RNA to a gene-expression analysis. In some embodiments, this includes the simultaneous analysis of multiple genes.

In analyses carried out to detect the presence or absence of RNA transcribed from a specific gene, the detection may be performed by any of a variety of physical, immunological and biochemical methods. Such methods are well-known in the art, and include, for example, protection from enzymatic degradation such as S1 analysis and RNase protection assays, in which hybridization to a labeled nucleic acid probe is followed by enzymatic degradation of single-stranded regions of the probe and analysis of the amount and length of probe protected from degradation.

In some embodiments of the invention, real time RT-PCR methods are employed that allow quantification of RNA transcripts and viewing of the increase in amount of nucleic acid as it is amplified. The TaqMan assay, a quenched fluorescent dye system, may also be used to quantitate targeted mRNA levels (see, for example K. J. Livak et al., PCR Methods Appl. 1995, 4: 357-362).

In some embodiments of the invention involving methods that allow quantification of RNA transcripts (such as real time RT-PCR), expression housekeeping genes are used as normalization controls. Examples of housekeeping genes include GAPDH, 18S rRNA, actin, tubulin, etc.

Other methods are based on the analysis of cDNA derived from mRNA, which is less sensitive to degradation than RNA and therefore easier to handle. These methods include, but are not limited to, sequencing cDNA inserts of an expressed sequence tag (EST) clone library (see, for example, M. D. Adams et al., Science, 1991, 252: 1651-1656) and serial analysis of gene expression (or SAGE), which allows quantitative and simultaneous analysis of a large number of transcripts (see, for example, U.S. Pat. No. 5,866,330; V. E. Velculescu et al., Science, 1995, 270: 484-487; and Zhang et al., Science, 1997, 276: 1268-1272). These two methods survey the whole spectrum of mRNA in a sample rather than focusing on a predetermined set.

Other methods of analysis of cDNA derived from mRNA include reverse transcriptase-mediated PCR (RT-PCR) gene expression assays. These methods are directed at specific target gene products and allow the qualitative (non-quantitative) detection of transcripts of very low abundance (see, for example, S. Su et al., BioTechniques, 1997, 22: 1107-1113). A variation of these methods, called competitive RT-PCR, in which a known amount of exogenous template is added as internal control, has been developed to allow quantitative measurements (see, for example, M. Beker-Andre and K. Hahlbrock, Nucl. Acids Res. 1989, 17: 9437-9346; A. M. Wang et al., Proc. Natl. Acad. Sci. USA, 1989, 86: 9717-9721; G. Gilliland et al., Proc. Natl. Acad. Sci. USA, 1990, 87: 2725-2729).

mRNA analysis may also be performed by differential display reverse transcriptase PCR (DDRT-PCR; see, for example, P. Liang and A. B. Pardee, Science, 1992, 257: 967-971) or RNA arbitrarily primed PCR (RAP-CPR; see, for example, J. Welsh et al., Nucl. Acids Res. 1992, 20: 4965-4970; and M. McClelland et al., EXS, 1993, 67: 103-115). In these methods, RT-PCR fingerprint profiles of transcripts are generated by random priming and differentially expressed genes appear as changes in the fingerprint profiles between two samples. Identification of a differentially expressed gene requires further manipulation (i.e., the appropriate band of the gel must be excised, subcloned, sequenced and matched to a gene in a sequence database).

iv. Array-Based Gene Expression Analysis of RNA from Brain Dendritic Cells

In certain embodiments, methods of the invention include submitting RNA to an array-based gene expression analysis.

Traditional molecular biology methods, such as most of those described above, typically assess one or a few genes per experiment, which significantly limits the overall throughput and prevents gaining a broad picture of gene function. Technologies based on DNA array or microarray (also called gene expression microarray), which were developed more recently, offer the advantage of allowing the monitoring of thousands of genes simultaneously through identification of sequence (gene/gene mutation) and determination of gene expression level (abundance) of genes (see, for example, A. Marshall and J. Hodgson, Nature Biotech. 1998, 16: 27-31; G. Ramsay, Nature Biotech. 1998, 16: 40-44; R. Ekins and R. W. Chu, Trends in Biotech. 1999, 17: 217-218; and D. J. Lockhart and E. A. Winzeler, Nature, 2000, 405: 827 836).

In a gene expression experiment, labeled cDNA or cRNA targets derived from the mRNA of an experimental sample are hybridized to nucleic acid probes immobilized to a solid support. By monitoring the amount of label associated with each DNA location, it is possible to infer the abundance of each mRNA species represented.

There are two standard types of DNA microarray technology in terms of the nature of the arrayed DNA sequence. In the first format, probe cDNA sequences (typically 500 to 5,000 bases long) are immobilized to a solid surface and exposed to a plurality of targets either separately or in a mixture. In the second format, oligonucleotides (typically 20-80-mer oligos) or peptide nucleic acid (PNA) probes are synthesized either in situ (i.e., directly on-chip) or by conventional synthesis followed by on-chip attachment, and then exposed to labeled samples of nucleic acids.

The analyzing step in such methods of the invention can be performed using any of a variety of methods, means and variations thereof for carrying out array-based gene expression analysis. Array-based gene expression methods are known in the art and have been described in numerous scientific publications as well as in patents (see, for example, M. Schena et al., Science, 1995, 270: 467-470; M. Schena et al., Proc. Natl. Acad. Sci. USA 1996, 93: 10614-10619; J. J. Chen et al., Genomics, 1998, 51: 313-324; U.S. Pat. Nos. 5,143,854; 5,445,934; 5,807,522; 5,837,832; 6,040,138; 6,045,996; 6,284,460; and 6,607,885)

In the practice of the present invention, these methods as well as other methods known in the art for carrying out array-based gene expression analysis may be used as described or modified such that they allow mRNA levels of gene expression to be evaluated.

### Test Sample

In some embodiments, RNA to be analyzed by an array-based gene expression method is isolated from brain dendritic cells as described herein. A test sample of RNA to be used in the methods of the invention may include a plurality of nucleic acid fragments labeled with a detectable agent.

The extracted brain dendritic cell RNA may be amplified, reverse-transcribed, labeled, fragmented, purified, concentrated and/or otherwise modified prior to the gene-expression analysis. Techniques for the manipulation of nucleic acids are well-known in the art, see, for example, J. Sambrook et al., “Molecular Cloning: A Laboratory Manual”, 1989, 2nd Ed., Cold Spring Harbour Laboratory Press: New York, N.Y.; “PCR Protocols: A Guide to Methods and Applications”, 1990, M. A. Innis (Ed.), Academic Press: New York, N.Y.; P. Tijssen “Hybridization with Nucleic Acid Probes—Laboratory Techniques in Biochemistry and Molecular Biology (Parts I and II)”, 1993, Elsevier Science; “PCR Strategies”, 1995, M. A. Innis (Ed.), Academic Press: New York, N.Y.; and “Short Protocols in Molecular Biology”, 2002, F. M. Ausubel (Ed.), 5th Ed., John Wiley & Sons.

In certain embodiments, in order to improve the resolution of the array-based gene expression analysis, the nucleic acid fragments of the test sample are less then 500 bases long, in some embodiments less than about 200 bases long. The use of small fragments significantly increases the reliability of the detection of small differences or the detection of unique sequences.

Methods of RNA fragmentation are known in the art and include: treatment with ribonucleases (e.g., RNase T1, RNase V1 and RNase A), sonication (see, for example, P. L. Deininger, Anal. Biochem. 1983, 129: 216-223), mechanical shearing, and the like (see, for example, J. Sambrook et al., “Molecular Cloning: A Laboratory Manual”, 1989, 2nd Ed., Cold Spring Harbour Laboratory Press: New York; P. Tijssen “Hybridization with Nucleic Acid Probes—Laboratory Techniques in Biochemistry and Molecular Biology (Parts I and II)”, 1993, Elsevier Science; C. P. Ordahl et al., Nucleic Acids Res. 1976, 3: 2985-2999; P. J. Oefner et al., Nucleic Acids Res. 1996, 24: 3879-3886; Y. R. Thorstenson et al., Genome Res. 1998, 8: 848-855). Random enzymatic digestion of the RNA leads to fragments containing as low as 25 to 30 bases.

Fragment size of the nucleic acid segments in the test sample may be evaluated by any of a variety of techniques, such as, for example, electrophoresis (see, for example, B. A. Siles and G. B. Collier, J. Chromatogr. A, 1997, 771: 319-329) or matrix-assisted laser desorption/ionization time-of-flight mass spectrometry (see, for example, N. H. Chiu et al., Nucl. Acids, Res. 2000, 28: E31).

In the practice of the methods of the invention, the test sample of RNA is labeled before analysis. Suitable methods of nucleic acid labeling with detectable agents have been described in detail above.

Prior to hybridization, labeled nucleic acid fragments of the test sample may be purified and concentrated before being resuspended in the hybridization buffer. Columns such as Microcon 30 columns may be used to purify and concentrate samples in a single step. Alternatively or additionally, nucleic acids may be purified using a membrane column (such as a Qiagen column) or Sephadex G50 and precipitated in the presence of ethanol.

### Gene-Expression Hybridization Arrays

Any of a variety of arrays may be used in the practice of the present invention. Investigators can either rely on commercially available arrays or generate their own. Methods of making and using arrays are well known in the art (see, for example, S. Kern and G. M. Hampton, Biotechniques, 1997, 23:120-124; M. Schummer et al., Biotechniques, 1997, 23:1087-1092; S. Solinas-Toldo et al., Genes, Chromosomes & Cancer, 1997, 20: 399-407; M. Johnston, Curr. Biol. 1998, 8: R171-R174; D. D. Bowtell, Nature Gen. 1999, Supp. 21:25-32; S. J. Watson and H. Akil, Biol Psychiatry. 1999, 45: 533-543; W. M. Freeman et al., Biotechniques. 2000, 29: 1042-1046 and 1048-1055; D. J. Lockhart and E. A. Winzeler, Nature, 2000, 405: 827-836; M. Cuzin, Transfus. Clin. Biol. 2001, 8:291-296; P. P. Zarrinkar et al., Genome Res. 2001, 11: 1256-1261; M. Gabig and G. Wegrzyn, Acta Biochim. Pol. 2001, 48: 615-622; and V. G. Cheung et al., Nature, 2001, 40: 953-958; see also, for example, U.S. Pat. Nos. 5,143,854; 5,434,049; 5,556,752; 5,632,957; 5,700,637; 5,744,305; 5,770,456; 5,800,992; 5,807,522; 5,830,645; 5,856,174; 5,959,098; 5,965,452; 6,013,440; 6,022,963; 6,045,996; 6,048,695; 6,054,270; 6,258,606; 6,261,776; 6,277,489; 6,277,628; 6,365,349; 6,387,626; 6,458,584; 6,503,711; 6,516,276; 6,521,465; 6,558,907; 6,562,565; 6,576,424; 6,587,579; 6,589,726; 6,594,432; 6,599,693; 6,600,031; and 6,613,893).

Arrays comprise a plurality of genetic probes immobilized to discrete spots (i.e., defined locations or assigned positions) on a substrate surface. Gene arrays used in accordance with some embodiments of the invention contain probes representing a comprehensive set of genes across the genome. In some such embodiments, the genes represented by the probes do not represent any particular subset of genes, and/or may be a random assortment of genes. In some embodiments of the invention, the gene arrays comprise a particular subset or subsets of genes. The subsets of genes may be represent particular classes of genes of interest. For example, an array comprising probes for neurodevelopmental genes may be used in order to focus analyses on neurodevelopmental genes. In such embodiments using arrays having particular subsets, more than one class of genes of interest may be represented on the same array.

Substrate surfaces suitable for use in the present invention can be made of any of a variety of rigid, semi-rigid or flexible materials that allow direct or indirect attachment (i.e., immobilization) of genetic probes to the substrate surface. Suitable materials include, but are not limited to: cellulose (see, for example, U.S. Pat. No. 5,068,269), cellulose acetate (see, for example, U.S. Pat. No. 6,048,457), nitrocellulose, glass (see, for example, U.S. Pat. No. 5,843,767), quartz or other crystalline substrates such as gallium arsenide, silicones (see, for example, U.S. Pat. No. 6,096,817), various plastics and plastic copolymers (see, for example, U.S. Pat. Nos. 4,355,153; 4,652,613; and 6,024,872), various membranes and gels (see, for example, U.S. Pat. No. 5,795,557), and paramagnetic or supramagnetic microparticles (see, for example, U.S. Pat. No. 5,939,261). When fluorescence is to be detected, arrays comprising cyclo-olefin polymers may in some embodiments be used (see, for example, U.S. Pat. No. 6,063,338).

The presence of reactive functional chemical groups (such as, for example, hydroxyl, carboxyl, amino groups and the like) on the material can be exploited to directly or indirectly attach genetic probes to the substrate surface. Methods for immobilizing genetic probes to substrate surfaces to form an array are well-known in the art.

More than one copy of each genetic probe may be spotted on an array (for example, in duplicate or in triplicate). This arrangement may, for example, allow assessment of the reproducibility of the results obtained. Related genetic probes may also be grouped in probe elements on an array. For example, a probe element may include a plurality of related genetic probes of different lengths but comprising substantially the same sequence. Alternatively, a probe element may include a plurality of related genetic probes that are fragments of different lengths resulting from digestion of more than one copy of a cloned piece of DNA. A probe element may also include a plurality of related genetic probes that are identical fragments except for the presence of a single base pair mismatch. An array may contain a plurality of probe elements. Probe elements on an array may be arranged on the substrate surface at different densities.

Array-immobilized genetic probes may be nucleic acids that contain sequences from genes (e.g., from a genomic library), including, for example, sequences that collectively cover a substantially complete genome or a subset of a genome (for example, the array may contain only human genes that are expressed throughout development). Genetic probes may be long cDNA sequences (500 to 5,000 bases long) or shorter sequences (for example, 20-80-mer oligonucleotides). Sequences of the genetic probes are those for which gene expression levels information is desired. Additionally or alternatively, the array may comprise nucleic acid sequences of unknown significance or location. Genetic probes may be used as positive or negative controls (for example, the nucleic acid sequences may be derived from karyotypically normal genomes or from genomes containing one or more chromosomal abnormalities; alternatively or additionally, the array may contain perfect match sequences as well as single base pair mismatch sequences to adjust for non-specific hybridization).

Techniques for the preparation and manipulation of genetic probes are well-known in the art (see, for example, J. Sambrook et al., “Molecular Cloning: A Laboratory Manual”, 1989, 2nd Ed., Cold Spring Harbour Laboratory Press: New York, N.Y.; “PCR Protocols: A Guide to Methods and Applications”, 1990, M. A. Innis (Ed.), Academic Press: New York, N.Y.; P. Tijssen “Hybridization with Nucleic Acid Probes—Laboratory Techniques in Biochemistry and Molecular Biology (Parts I and II)”, 1993, Elsevier Science; “PCR Strategies”, 1995, M. A. Innis (Ed.), Academic Press: New York, N.Y.; and “Short Protocols in Molecular Biology”, 2002, F. M. Ausubel (Ed.), 5th Ed., John Wiley & Sons).

Long cDNA sequences may be obtained and manipulated by cloning into various vehicles. They may be screened and re-cloned or amplified from any source of genomic DNA. Genetic probes may be derived from genomic clones including mammalian and human artificial chromosomes (MACs and HACs, respectively, which can contain inserts from ˜5 to 400 kilobases (kb)), satellite artificial chromosomes or satellite DNA-based artificial chromosomes (SATACs), yeast artificial chromosomes (YACs; 0.2-1 Mb in size), bacterial artificial chromosomes (BACs; up to 300 kb); P1 artificial chromosomes (PACs; ˜70-100 kb) and the like.

Genetic probes may also be obtained and manipulated by cloning into other cloning vehicles such as, for example, recombinant viruses, cosmids, or plasmids (see, for example, U.S. Pat. Nos. 5,266,489; 5,288,641 and 5,501,979).

In some embodiments, genetic probes are synthesized in vitro by chemical techniques well-known in the art and then immobilized on arrays. Such methods are especially suitable for obtaining genetic probes comprising short sequences such as oligonucleotides and have been described in scientific articles as well as in patents (see, for example, S. A. Narang et al., Meth. Enzymol. 1979, 68: 90-98; E. L. Brown et al., Meth. Enzymol. 1979, 68: 109-151; E. S. Belousov et al., Nucleic Acids Res. 1997, 25: 3440-3444; D. Guschin et al., Anal. Biochem. 1997, 250: 203-211; M. J. Blommers et al., Biochemistry, 1994, 33: 7886-7896; and K. Frenkel et al., Free Radic. Biol. Med. 1995, 19: 373-380; see also for example, U.S. Pat. No. 4,458,066).

For example, oligonucleotides may be prepared using an automated, solid-phase procedure based on the phosphoramidite approach. In such a method, each nucleotide is individually added to the 5-end of the growing oligonucleotide chain, which is attached at the 3′-end to a solid support. The added nucleotides are in the form of trivalent 3′ phosphoramidites that are protected from polymerization by a dimethoxytrityl (or DMT) group at the 5-position. After base-induced phosphoramidite coupling, mild oxidation to give a pentavalent phosphotriester intermediate and DMT removal provides a new site for oligonucleotide elongation. The oligonucleotides are then cleaved off the solid support, and the phosphodiester and exocyclic amino groups are deprotected with ammonium hydroxide. These syntheses may be performed on commercial oligo synthesizers such as the Perkin Elmer/Applied Biosystems Division DNA synthesizer.

Methods of attachment (or immobilization) of oligonucleotides on substrate supports have been described (see, for example, U. Maskos and E. M. Southern, Nucleic Acids Res. 1992, 20: 1679-1684; R. S. Matson et al., Anal. Biochem. 1995, 224; 110-116; R. J. Lipshutz et al., Nat. Genet. 1999, 21: 20-24; Y. H. Rogers et al., Anal. Biochem. 1999, 266: 23-30; M. A. Podyminogin et al., Nucleic Acids Res. 2001, 29: 5090-5098; Y. Belosludtsev et al., Anal. Biochem. 2001, 292: 250-256).

Oligonucleotide-based arrays have also been prepared by synthesis in situ using a combination of photolithography and oligonucleotide chemistry (see, for example, A. C. Pease et al., Proc. Natl. Acad. Sci. USA 1994, 91: 5022-5026; D. J. Lockhart et al., Nature Biotech. 1996, 14: 1675-1680; S. Singh-Gasson et al., Nat. Biotechn. 1999, 17: 974-978; M. C. Pirrung et al., Org. Lett. 2001, 3: 1105-1108; G. H. McGall et al., Methods Mol. Biol. 2001, 170; 71-101; A. D. Barone et al., Nucleosides Nucleotides Nucleic Acids, 2001, 20: 525-531; J. H. Butler et al., J. Am. Chem. Soc. 2001, 123: 8887-8894; E. F. Nuwaysir et al., Genome Res. 2002, 12: 1749-1755). The chemistry for light-directed oligonucleotide synthesis using photolabile protected 2′-deoxynucleoside phosphoramites has been developed by Affymetrix Inc. (Santa Clara, Calif.) and is well known in the art (see, for example, U.S. Pat. Nos. 5,424,186 and 6,582,908).

An alternative to custom arraying of genetic probes is to rely on commercially available arrays and micro-arrays. Such arrays have been developed, for example, by Affymetrix Inc. (Santa Clara, Calif.), Illumina, Inc. (San Diego, Calif.), Spectral Genomics, Inc. (Houston, Tex.), and Vysis Corporation (Downers Grove, Ill.).

### Hybridization

In certain methods of the invention, a gene expression array may be contacted with a test sample under conditions wherein nucleic acid fragments in the sample specifically hybridize to the genetic probes immobilized on the array.

Hybridization reaction(s) and washing step(s), if any, may be carried out under any of a variety of experimental conditions. Numerous hybridization and wash protocols have been described and are well-known in the art (see, for example, J. Sambrook et al., “Molecular Cloning: A Laboratory Manual”, 1989, 2nd Ed., Cold Spring Harbour Laboratory Press: New York; P. Tijssen “Hybridization with Nucleic Acid Probes—Laboratory Techniques in Biochemistry and Molecular Biology (Part II)”, Elsevier Science, 1993; and “Nucleic Acid Hybridization”, M. L. M. Anderson (Ed.), 1999, Springer Verlag: New York, N.Y.). Methods of the invention may be carried out by following known hybridization protocols, by using modified or optimized versions of known hybridization protocols or newly developed hybridization protocols as long as these protocols allow specific hybridization to take place.

The term “specific hybridization” refers to a process in which a nucleic acid molecule preferentially binds, duplexes, or hybridizes to a particular nucleic acid sequence under stringent conditions. In the context of the present invention, this term more specifically refers to a process in which a nucleic acid fragment from a test sample preferentially binds (i.e., hybridizes) to a particular genetic probe immobilized on the array and to a lesser extent, or not at all, to other immobilized genetic probes of the array. Stringent hybridization conditions are sequence dependent. The specificity of hybridization increases with the stringency of the hybridization conditions; reducing the stringency of the hybridization conditions results in a higher degree of mismatch being tolerated.

Hybridization and/or wash conditions may be adjusted by varying different factors such as the hybridization reaction time, the time of the washing step(s), the temperature of the hybridization reaction and/or of the washing process, the components of the hybridization and/or wash buffers, the concentrations of these components as well as the pH and ionic strength of the hybridization and/or wash buffers.

In certain embodiments, hybridization and/or wash steps are carried out under very stringent conditions. In other embodiments, hybridization and/or wash steps are carried out under moderate to stringent conditions. In still other embodiments, more than one washing steps are performed. For example, in order to reduce background signal, a medium to low stringency wash is followed by a wash carried out under very stringent conditions.

As is well known in the art, hybridization processes may often be enhanced by modifying other reaction conditions. For example, the efficiency of hybridization (i.e., time to equilibrium) may be enhanced by using reaction conditions that include temperature fluctuations (i.e., differences in temperature that are higher than a couple of degrees). An oven or other devices capable of generating variations in temperatures may be used in the practice of the methods of the invention to obtain temperature fluctuation conditions during the hybridization process.

It is also known in the art that hybridization efficiency can often be significantly improved if the reaction takes place in an environment where the humidity is not saturated. Controlling the humidity during the hybridization process provides another means to increase the hybridization sensitivity. Array-based instruments usually include housings allowing control of the humidity during all the different stages of the experiment (i.e., pre-hybridization, hybridization, wash and detection steps).

Additionally or alternatively, a hybridization environment that includes osmotic fluctuation may be used to increase hybridization efficiency. Such an environment where the hyper-/hypo-tonicity of the hybridization reaction mixture varies may be obtained by creating a solute gradient in the hybridization chamber, for example, by placing a hybridization buffer containing a low salt concentration on one side of the chamber and a hybridization buffer containing a higher salt concentration on the other side of the chamber

### Highly Repetitive Sequences

In the practice of the methods of the invention, an array may be contacted with a test sample under conditions wherein nucleic acid segments in the sample specifically hybridize to genetic probes on the array. As mentioned above, the selection of appropriate hybridization conditions will allow specific hybridization to take place. In certain cases, the specificity of hybridization may further be enhanced by inhibiting repetitive sequences.

In certain embodiments, repetitive sequences present in the nucleic acid fragments are removed or their hybridization capacity is disabled. By excluding repetitive sequences from the hybridization reaction or by suppressing their hybridization capacity, one prevents the signal from hybridized nucleic acids to be dominated by the signal originating from these repetitive-type sequences (which are statistically more likely to undergo hybridization). Failure to remove repetitive sequences from the hybridization or to suppress their hybridization capacity results in non-specific hybridization, making it difficult to distinguish the signal from the background noise.

Removing repetitive sequences from a mixture or disabling their hybridization capacity can be accomplished using any of a variety of methods well-known to those skilled in the art. Such methods include, but are not limited to, removing repetitive sequences by hybridization to specific nucleic acid sequences immobilized to a solid support (see, for example, O. Brison et al., Mol. Cell. Biol. 1982, 2: 578-587); suppressing the production of repetitive sequences by PCR amplification using adequate PCR primers; or inhibiting the hybridization capacity of highly repeated sequences by self-reassociation (see, for example, R. J. Britten et al., Methods of Enzymol., 1974, 29: 363-418).

In some embodiments, the hybridization capacity of highly repeated sequences is competitively inhibited by including, in the hybridization mixture, unlabeled blocking nucleic acids. Such unlabeled blocking nucleic acids, which are mixed to the test sample before the contacting step, act as a competitor and prevent the labeled repetitive sequences from binding to the highly repetitive sequences of the genetic probes, thus decreasing hybridization background. In certain embodiments, for example when cDNA derived from mRNA is analyzed, the unlabeled blocking nucleic acids are Human Cot-1 DNA. Human Cot-1 DNA is commercially available, for example, from Gibco/BRL Life Technologies (Gaithersburg, Md.).

### Binding Detection and Data Analysis

In some embodiments, inventive methods include determining the binding of individual nucleic acid fragments of the test sample to individual genetic probes immobilized on the array in order to obtain a binding pattern. In array-based gene expression, determination of the binding pattern is carried out by analyzing the labeled array which results from hybridization of labeled nucleic acid segments to immobilized genetic probes.

In certain embodiments, determination of the binding includes: measuring the intensity of the signals produced by the detectable agent at each discrete spot on the array.

Analysis of the labeled array may be carried out using any of a variety of means and methods, whose selection will depend on the nature of the detectable agent and the detection system of the array-based instrument used.

In certain embodiments, the detectable agent comprises a fluorescent dye and the binding is detected by fluorescence. In other embodiments, the sample of RNA is biotin-labeled and after hybridization to immobilized genetic probes, the hybridization products are stained with a streptavidin-phycoerythrin conjugate and visualized by fluorescence. Analysis of a fluorescently labeled array usually comprises: detection of fluorescence over the whole array, image acquisition, quantitation of fluorescence intensity from the imaged array, and data analysis.

Methods for the detection of fluorescent labels and the creation of fluorescence images are well known in the art and include the use of “array reading” or “scanning” systems, such as charge-coupled devices (i.e., CCDs). Any known device or method, or variation thereof can be used or adapted to practice the methods of the invention (see, for example, Y. Hiraoka et al., Science, 1987, 238: 36-41; R. S. Aikens et al., Meth. Cell Biol. 1989, 29: 291-313; A. Divane et al., Prenat. Diagn. 1994, 14: 1061-1069; S. M. Jalal et al., Mayo Clin. Proc. 1998, 73: 132-137; V. G. Cheung et al., Nature Genet. 1999, 21: 15-19; see also, for example, U.S. Pat. Nos. 5,539,517; 5,790,727; 5,846,708; 5,880,473; 5,922,617; 5,943,129; 6,049,380; 6,054,279; 6,055,325; 6,066,459; 6,140,044; 6,143,495; 6,191,425; 6,252,664; 6,261,776 and 6,294,331).

Commercially available microarrays scanners are typically laser-based scanning systems that can acquire one (or more) fluorescent image (such as, for example, the instruments commercially available from PerkinElmer Life and Analytical Sciences, Inc. (Boston, Mass.), Virtek Vision, Inc. (Ontario, Canada) and Axon Instruments, Inc. (Union City, Calif.)). Arrays can be scanned using different laser intensities in order to ensure the detection of weak fluorescence signals and the linearity of the signal response at each spot on the array. Fluorochrome-specific optical filters may be used during the acquisition of the fluorescent images. Filter sets are commercially available, for example, from Chroma Technology Corp. (Rockingham, Vt.).

In some embodiments, a computer-assisted imaging system capable of generating and acquiring fluorescence images from arrays such as those described above, is used in the practice of the methods of the invention. One or more fluorescent images of the labeled array after hybridization may be acquired and stored.

In some embodiments, a computer-assisted image analysis system is used to analyze the acquired fluorescent images. Such systems allow for an accurate quantitation of the intensity differences and for an easier interpretation of the results. A software for fluorescence quantitation and fluorescence ratio determination at discrete spots on an array is usually included with the scanner hardware. Softwares and/or hardwares are commercially available and may be obtained from, for example, BioDiscovery (El Segundo, Calif.), Imaging Research (Ontario, Canada), Affymetrix, Inc. (Santa Clara, Calif.), Applied Spectral Imaging Inc. (Carlsbad, Calif.); Chroma Technology Corp. (Brattleboro, Vt.); Leica Microsystems, (Bannockburn, Ill.); and Vysis Inc. (Downers Grove, Ill.). Other softwares are publicly available (e.g., MicroArray Image Analysis, and Combined Expression Data and Sequence Analysis (http://rana.lbl.gov); D. Y. Chiang et al., Bioinformatics, 2001, 17: 49-55; a system written in R and available through the Bioconductor project (http://www.bioconductor.org); a Java-based TM4 software system available from the Institute for Genomic Research (http://www.tigr.org/software); and a Web-based system developed at Lund University (http://base.thep.lu.Se)).

Accurate determination of fluorescence intensities requires normalization and determination of the fluorescence ratio baseline (A. Brazma and J. Vilo, FEBS Lett. 2000, 480: 17-24). Data reproducibility may be assessed by using arrays on which genetic probes are spotted in duplicate or triplicate. Baseline thresholds may also be determined using global normalization approaches (M. K. Kerr et al., J. Comput. Biol. 2000, 7: 819-837). Other arrays include a set of maintenance genes which shows consistent levels of expression over a wide variety of tissues and allows the normalization and scaling of array experiments.

In the practice of the methods of the invention, any of a large variety of bioinformatics and statistical methods may be used to analyze data obtained by array-based gene expression analysis. Such methods are well known in the art (for a review of essential elements of data acquisition, data processing, data analysis, data mining and of the quality, relevance and validation of information extracted by different bioinformatics and statistical methods, see, for example, A. Watson et al., Curr. Opin. Biotechnol. 1998, 9: 609-614; D. J. Duggan et al., Nat. Genet. 1999, 21: 10-14; D. E. Bassett et al., Nat. Genet. 1999, 21: 51-55; K. R. Hess et al., Trends Biotechnol. 2001, 19: 463-468; E. Marcotte and S. Date, Brief Bioinform. 2001, 2: 363-374; J. N. Weinstein et al., Cytometry, 2002, 47: 46-49; T. G. Dewey, Drug Discov. Today, 2002, 7: S170-S175; A Butte, Nat. Rev. Drug Discov. 2002, 1: 951-960; J. Tamames et al., J. Biotechnol. 2002, 98: 269-283; Z Xiang et al., Curr. Opin. Drug Discov. Devel. 2003, 6: 384-395.

v. Control Samples

For comparing gene expression in brain dendritic cells, control samples may, for example, be derived from certain cell type, populations of cells, tissues etc. that are substantially free of brain dendritic cells. For example, microglia that have a marker phenotype distinguishable from that of brain dendritic cells may be used as a control sample. In some embodiments, the control sample is derived from the same animal as the brain dendritic cells. In some embodiments, the control sample is sorted from a population of cells (that may be, for example, obtained from brain tissue) that contains brain dendritic cells and other cells. For example, brain cells from CD11c-EYFP+ transgenic mice may be sorted into EYFP+ brain dendritic cell and EYFP− microglia subpopulations. The EYFP− microglia may be used as a control sample for the EYFP+ brain dendritic cell.

In some embodiments, control samples are processed in parallel with samples from brain dendritic cells. Such parallel processing may enable more accurate gene expression comparisons.

In some embodiments, control samples are processed in experiments separate from experiments performed to process RNA from brain dendritic cells. For example, gene expression data from control samples may be archived for comparison against gene expression data later obtained from brain dendritic cells.

vi. Gene Expression Levels and Determining Genes that are Differentially Expressed.

Methods of determining levels of gene expression have already been described herein. In gene expression array experiments, quantitative readouts of expression levels are typically provided. Typically, after normalization of data, genes having at least a 2-fold differences (i.e. a ratio of about 2) in expression levels between test and control samples may be considered “differentially expressed.” In some embodiments of the invention, genes considered to be differentially expressed show at least two-fold, at least five-fold, at least ten-fold, at least 15-fold, at least 20-fold, or at least 25-fold different expression levels compared to controls. (It is to be understood that the fold different expression levels can be determined in either direction, i.e., the expression levels for the test sample may be at least 2-fold higher or 2-fold lower than expression levels for the control sample.)

It will be appreciated, however, that both the fold-difference cutoff for being considered differentially expressed varies depending on several factors which may include, for example, the type of samples used, the quantity and quality of the RNA sample, the power of the statistical analyses, the type of genes of interest, etc. In some embodiments, a lower cutoff ratio (i.e. -fold difference) is used, e.g., ratios of about 1.8, or about 1.6. In some embodiments, a higher cutoff ratio than about 2.0 is used, e.g., about 2.5, about 3.0, about 3.5, about 4.0, about 4.5, about 5.0, etc.

In some embodiments of the invention, a preliminary list of genes is identified as being differentially expressed using a particular statistical method or particular set of experimental data. In some embodiments, the preliminary list is narrowed down by identifying genes of interest or of particular fold differences within the list. Determining which genes among the preliminary list may be done in a hypothesis-driven manner. For example, only genes on the preliminary list that are deemed to be physiologically relevant (as determined, by example, by what is known of the gene's function, localization, structure, etc.) may be ultimately identified as differentially expressed genes of interest. In some embodiments, genes are identified within the preliminary list without regard to a particular hypothesis. A subset of genes from the preliminary list may be identified as genes of interest using, for example, a different method of gene expression analysis, a different set of samples. etc. In some embodiments, no further selection or identification of genes is done after obtaining the preliminary list of genes.

It will be understood that inventive methods may identify some genes that are not known, not previously described in the literature, and/or not catalogued in publicly available databases. For example, some gene expression microarrays may contain probes for genes that have not yet been characterized or known in the literature. In cases in which uncharacterized genes are identified as being differentially regulated, the genes may still be described as being “identified” because there is usually an identifier, e.g., a probe with a known sequence on the microarray that can be associated with the gene, a name of an expressed sequence tag, etc.

### V. Genes Involved in Neurological Diseases

In another aspect, the invention provides methods for identifying genes involved in a neurological disease. Such methods for example, may comprise a step of detecting or identifying one or more genes that are differentially regulated in an animal that is a model for a neurodegenerative disease and that contains detectably labeled brain dendritic cells as compared to a control sample, wherein the control sample comprises RNA obtained from cells from an animal that is not a model for the neurological disease.

Animals such as transgenic animals that are labeled with a detectable agent may be as described above. For example, CD11c-EYFP transgenic mice, in which an EYFP transgene is under the control of a CD11c promoter, may be used in the practice of these inventive methods. Steps of isolating, obtaining RNA, and detecting or identifying genes may also be performed by methods such as those previously described herein.

### Models for Neurological Disease

Brain dendritic cells can be studied in the context of a particular disease by studying bDC in an neurological disease model. A variety of animal disease models can be used in accordance with the invention; particularly suitable disease models include neurodevelopmental and neurodegenerative disease models.

For example, a variety of Alzheimer's Disease models exist. (For a discussion of such models, see the website for the Jackson Laboratory, ‘http://’ followed immediately by ‘jaxmicejax.org.’) For example, transgenic mice expressing amyloid precursor protein (such as, for example, human amyloid beta (A4) precursor protein APP containing the Familial Alzheimer's Disease (FAD) Swedish mutation K670N/M671L and/or the Indiana (V717F) mutations (APPSwInd)) may be used. Other transgenic models include mice bearing transgenes for mutant or wild type versions of presenilin (such as, for example, PS1-dE9), microtubule-associated protein tau (MAPT), human apolipoprotein isoforms (Apoe^{tmlUnc} mutation, APOE2, APOE3, APOE4, etc.), β-secretase (BACE, Asp2, Mempasin, etc.), etc. Double transgenic mice, for example, those expressing amyloid precursor protein and a mutant presenilin 1 in central nervous system neurons may be used as models for Alzheimer's disease. In some models of Alzheimer's disease, a chimeric mouse/human amyloid precursor protein such as Mo/HuAPP695swe is used.

A variety of promoters can be used to direct expression of transgenes to CNS neurons, such as neural-specific elements of the Thyl promoter, the promoter for the prion gene (Prnp), etc. In some models, promoters directing transgenes to glia may be desirable. For example, the glial fibrillary acid protein (GFAP) promoter may be used to direct expression of apolipoprotein to glia. Inducilble promoters may also be used, allowing one to induce Alzheimer's Disease. Suitable inducible promoters include tetracycline-responsive promoter element.

As another example, Huntington's Disease animal models may also be employed in the practice of the invention. (For a discussion of Huntington's Disease models, see, for example, Menalled and Chesselet, 2002. “Mouse models of Huntington's disease.”Trends Pharmacol Sci. 23(1):32-9, the entire contents of which are herein incorporated by reference.) Huntington's Disease models include transgenic and knock-in mice which include transgenic and/or mutant forms of the Hdh gene (which encodes huntingtin).

As another example, Parkinson's Disease animal models may be used. (For a discussion of such models, see the website for the Jackson Laboratory, ‘http://’ followed immediately by ‘jaxmicejax.org.’). For example, synuclein mutant mice are often used as a model for Parkinson's Disease.

Disease models of multiple sclerosis also exist. For example, experimental allergic encephalitis may be induced in mice, guinea pigs, etc. to mimic multiple sclerosis.

A disease model strain may be crossed together with an animal carrying a transgene for a detectable agent that labels brain dendritic cells. Progeny of such crosses may carry both the detectable marker and the relevant transgene(s) and/or gene mutations that cause it to be a model for a particular disease. For example a CD11c-YFP transgenic mouse can be crossed to an amyloid precursor protein transgenic/mutant mouse.

### VI. Modulators of Brain Dendritic Cells

In another aspect, the invention provides methods of identifying agents that modulate brain dendritic cells. Such methods generally comprise steps of providing a sample that contains brain dendritic cells; contacting the sample with a test agent; determining whether the test agent modulates one or more aspects of brain dendritic cell development, activity, gene expression, and localization; and identifying, based on the determination, that the test agent as a modulator of brain dendritic cells.

Test agents may be found in compound libraries such as those from historical collections of compounds and/or libraries from diversity-oriented syntheses may be screened with inventive methods. Such parallel assays may allow identification of a compound that modulates brain dendritic cells.

Modulation of brain dendritic cell can comprise altering morphology, marker expression, gene expression, ability to present antigen, localization, migration, etc. of brain dendritic cells. For example, expression of gene products involved in antigen presentation (such as MHC (major histocompatibility complex) molecules like MHC class II), cytokines (such as TNFα, IL-6, nitric oxide, and combinations thereof), cytokine receptors, etc., may be altered. Expression of combinations of such gene products may be altered.

Other genes whose expression may be altered include, but are not limited to, resistin-like alpha, CCL17, CxCL9, CD209a (DC-Sign), H2-Eb1, Spp1 (Osteopontin), Axl, H2-Aa, H2-Al, CxCl2 (MIP-2), Clec7a (Dectin-1), CCR2, Itgax (CD11c), IGF-1, CD36, etc.

Responses to cytokines may be altered. For example, interfereon γ (INFγ) induces activation of brain dendritic cells and expression of MHC class T1 molecules; such an induction may be altered by a modulator of brain dendritic cells.

### EXAMPLES

The following examples describe some of the preferred modes of making and practicing the present invention. However, it should be understood that these examples are for illustrative purposes only and are not meant to limit the scope of the invention. Furthermore, unless the description in an Example is presented in the past tense, the text, like the rest of the specification, is not intended to suggest that experiments were actually performed or data were actually obtained.

### Example 1

### Phenotype of Brain Dendritic Cells

### Materials and Methods

### Animals

All mice used in the following Examples were treated in accordance with protocols approved by the Institutional Animal Care and Use Committee at The Rockefeller University.

### Transgenic Animals

Transgenic Itgax (CD11c)-EYFP mice were developed by the Nussenzweig Laboratory at The Rockefeller University and have been used to identify dendritic cells in lymphoid tissues in the steady state (Lindquist et al., 2004). The CD11c-EYFP transgene was generated by cloning PCR-amplified EYFP-venus DNA (Nagai et al., 2002) as an EcoR I fragment into the CD11c-pDOI-5 vector (Brocker et al., [1997]). A linearized CD11c-EYFP construct was isolated following Sal I and Not I digestion and injected into C57BL/6-CBA F1 fertilized female pronuclei. Progeny positive by PCR for the transgene were back-crossed to C57BL/6 for at least 10 generations. Itgax (CD11c)-EYFP transgenic mice were transferred to the Harold and Margaret Milliken Hatch Laboratory of Neuroendocrinology facilities for propagation. CD11c-EYFP-positive mice were genotyped for the presence of CD11c-EYFP transgene by PCR.

The transgenic mouse line p7.2fms harbors an EGFP transgene under the control of the CSF-1R promoter and was obtained from Dr. Jeffrey Pollard with permission from Dr. David Hume from the Transgenic Animal Service of Queensland, Brisbane, Queensland, Australia. Construction of this line has been described elsewhere (Sasmono et al., 2003). The CSF-1R-EGFP transgene was generated by replacing the luciferase gene in the vector pGL6.7fms (Yue et al., 1993) with a green fluorescent protein gene derived from the pEGFP—N1 vector (Clontech, Mountain View, Calif.). The cfms gene encodes the receptor for macrophage colony-stimulating factor (CSF-1); driving expression of EGFP using the cfms promoter results in EGFP expression in cells of the mononuclear phagocytic lineage, including microglia. The inventors have used this mouse in previous studies to visualize microglia in the CNS (Sierra et al., 2007).

Transgenic colonies were bred and maintained at the Rockefeller University facilities in a pathogen-free environment under 12:12-hour light/dark cycle with ad libitum access to chow and water. EYFP-positive brains used in anatomic experiments were derived from E10, E16 (E0=day of conception); PN1, PN2, and PN8 (where PN1=day of birth); and adult males 6 weeks to 3 months old. PN2 mouse brains were used in tissue culture experiments to identify CD11c protein in FACS EYFP+ bDC. Adult male mice aged 6 weeks to 3 months (N=47) were used in ex vivo FACS experiments to evaluate Itgax mRNA in EYFP+ bDC.

### Perfusion, Sectioning, and Tissue Storage for Light and Electron Microscopy

Perfusion protocols were tailored to optimize antibodies and techniques used in this study. For light microscopy, mice were deeply anesthetized with sodium pentobarbital (150 mg/kg, i.p.), followed by sequential intracardial perfusion with saline containing heparin (1 U/mL) and 4% paraformaldehyde in 0.1 M PB. Brains were removed and immersed in the same fixative for 4 hours at 4° C., rinsed in PBS, and incubated in 30% sucrose until they submerged. Perfused brains were rinsed and 40 μm serial sections were cut on a vibratome (Leica, Wetzlar, Germany). Embryos were removed from lethally anesthetized females on days 10 and 16 of gestation and quickly frozen. Twenty-micrometer serial sagittal sections were cut and collected on Vectabond-coated slides (Vector Laboratories, Burlingame, Calif.). PN1 and PN2 pups were ice-anesthetized and P8 pups were decapitated, then brains were quickly frozen. Twenty-micrometer serial coronal sections were cut and collected on Vectabond-coated slides. Slides were stored at −80° C. until ready to use.

For fluorescence microscopy, perfusions for adult mice were performed as described above. Tissues were then frozen, and 40 μm coronal serial sections were cut on a Leica vibratome or on a sliding freezing microtome (Microm, Walldorf, Germany). Serial sections were collected and stored at −20° C. in cryoprotectant (25% ethylene glycol, 25% glycerol in 0.1 M PB, pH 7.4) until use. Brains used to identify cell membrane antigens were removed from decapitated mice and immediately frozen. Forty-micrometer serial coronal sections were cut and collected on Vectabond-coated slides and stored at −20° C.

For ultrastructural analysis, mice were deeply anesthetized with sodium pentobarbital (150 mg/kg, i.p.), followed by sequential intracardial perfusion with 10 mL heparin (1,000 U/mL) in normal saline and 40 mL acrolein (3.75%; Polysciences, Warrington, Pa.) in 2% paraformaldehyde in 0.1 M PB (pH 7.4). Brains were removed, cut into 4-5 mm coronal blocks, and postfixed for 30 min. Sections (40 μm thick) were cut through the brain and spinal cord on a Leica vibratome, collected in PB, and stored in cryoprotectant until use.

### Antibodies

Antibodies used for immunohistochemical characterization studies are listed in Table 1 and described below.

Monoclonal rat anti-CD11b (integrin alpha M, Mac-1) was produced using mouse thioglycollate-stimulated peritoneal macrophages (Serotec, Raleigh, N.C.; clone 5C6; MCA711) as the antigen. This antibody recognizes the mouse complement type 3 receptor and precipitates a heterodimer of approximately 165-170 and approximately 95 kD on Western blots (Rosen and Gordon, 1987).

Rat anti-CD11b (integrin alpha M, Mac-1 alpha)-Alexa Fluor 647 antibody was produced using B 10 mouse activated splenic cells (eBioscience, San Diego, Calif.; 51-0112) as the antigen. This monoclonal antibody (clone M1/70) similarly detects a heterodimer of approximately 165-170 and approximately 95 kD on Western blots (Sanchez-Madrid et al., 1983) and was used for FACS analyses.

Rat anti-F4/80 antibody was produced using mouse thioglycollate-stimulated peritoneal macrophages (Serotec; MCA497R) as the antigen. This monoclonal antibody (clone Cl:A3-1) precipitates an approximately 160 kD cell surface glycoprotein (Austyn and Gordon, 1981) corresponding to the murine F4/80 antigen and labels macrophages in tissues of wild-type but not F4/80−/− mice (Lin et al., 2005).

Rabbit anti-Iba-1 polyclonal antibody was produced using a synthetic peptide corresponding to the C-terminus of rat Iba-1 (amino acid residues 134-147; Wako Chemicals, Richmond, Va.; 019-19741) as the antigen. This antibody recognizes a single band approximately 17 kD in size on Western blots and labels microglia and macrophages but not neurons or astrocytes (Ito et al., 1998).

The monoclonal mouse anti-NeuN antibody (clone A60; Chemicon, Temucella, Calif.; MAB377) was produced using purified cell nuclei from mouse brain. NeuN is a neuron-specific protein present in most CNS and PNS neuronal cell types of all vertebrates tested (see manufacturer's technical data sheet for list of species; Mullen et al., 1992). This antibody recognizes four bands on Western blots (Unal-Cevik et al., 2004). The four bands are thought to reflect multiple phosphorylation states of NeuN (Lind et al., 2005).

Affinity purified polyclonal goat anti-Dcx antibody (Santa Cruz Biotechnology, Santa Cruz, Calif.; SC-8066) was produced using a peptide sequence corresponding to amino acid residues 385-402 of human Dcx. This antiserum recognizes mouse, rat, and human developing neurons (Farrar et al., 2005; manufacturer's technical information) and precipitates a single band of approximately 40 kD, consistent with the molecular weight of the Dcx protein on Western blots (Gleeson et al., 1998; Brown et al., 2003).

Affinity-purified goat anti-GFAP antibody (C-19) was produced using a peptide corresponding to human GFAP (amino acid residues 382-432, which are located in the C-terminus) as the stimulating antigen (Santa Cruz Biotechnology; SC-6170). This antibody reacts with GFAP-containing astrocytes of mouse, rat, and human origin and detects a single band of approximately 50 kD by Western blot (Chang et al., 2001; manufacturer's technical information).

Rabbit anti-NG2 antibody was produced using purified NG2 (Chemicon; AB5320). This polyclonal antibody recognizes oligodendrocytes in brain sections and identifies both the intact proteoglycan (500 kD) and the core protein (300 kD) by Western blot and ELISA (Stallcup et al., 1990; Chemicon materials data sheet; see the website at the address ‘www.’ followed immediately by ‘chemiconcustom.com’).

Chicken anti-GFP antibody was generated against recombinant GFP (Aves Lab Inc, San Diego, Calif.; GFP-1020) and recognizes GFP gene product from GFP transgenic mice (Encinas et al., 2006). Specificity has been verified by Western blot (see data sheet for GFP-1020 at the web address ‘www.’ followed immediately by ‘aveslab.com’).

Alexa Fluor 647 Armenian hamster anti-CD11c (Integrin alpha x) antibody was produced using mouse spleen dendritic cells (eBioscience; 51-0114) and was used for FACS analyses. This monoclonal antibody (clone N418) reacts with mouse CD11c, the integrin alpha x, and precipitates an approximately 150 and approximately 90 kDa heterodimer (CD11c/CD18; Metlay et al., 1990). Isotype-hamster IgG-APC (clone eBio299Arm) for CD11c served as a control.

Armenian hamster anti-CD11c MAb (p150/95 chain) antibody from Pierce (Rockford, Ill.; MA11C5) is the unconjugated form of the N418 clone.

TABLE 1
Antibodies used in Examples

| Primary antibody | Immunogen | Dilution | Company | Catalog No. |
|---|---|---|---|---|
| Rat anti-CD11b | Mouse thioglycollate | 1:1,000 | Serotec, | MCA711 |
| (Mac-1) | stimulated peritoneal | | Raleigh, NC | |
| | macrophages | | | |
| Alexa Fluor 647 | B10 mouse spleen cells | 1:200 | eBioscience, | 51-0112 |
| rat anti-CD11b | enriched for T cells | (FACS) | San Diego, CA | |
| (integrin alpha | | | | |
| M, Mac-1 alpha) | | | | |
| Rat anti-F4/80 | Mouse thioglycollate | 1:500 | Serotec, | MCA497R |
| | stimulated peritoneal | | Raleigh, NC | |
| | macrophages | | | |
| Rabbit anti-Iba-1 | Synthetic peptide | 1:2,000 | Wako | 019-19741 |
| | corresponding to the C- | | Chemicals, | |
| | terminus of rat Iba-1 | | Richmond, VA | |
| | (aa 134-147) | | | |
| Mouse anti-NeuN | Purified rat cell nuclei | 1:1,000 | Chemicon, | MAB377 |
| | | | Temecula, CA | |
| Goat anti-Dcx | Peptide corresponding to the | 1:500 | Santa Cruz, CA | SC-8066 |
| | C-terminus of human Dcx | | | |
| | (aa 385-402) | | | |
| Mouse anti- | Peptide corresponding to the | 1:1,000 | Santa Cruz, CA | SC-6170 |
| GFAP | C-terminus of human GFAP | | | |
| Rabbit anti-NG2 | Purified NG2Rat NG2 | 1:200 | Chemicon, | AB5320 |
| (chondroitin | Chondroitin Sulfate | | Temecula, CA | |
| sulfate | Proteoglycan | | | |
| proteoglycan) | | | | |
| Chicken anti- | Recombinant GFP | 1:2,000- | Aves Lab Inc., | GFP-1020 |
| GFP | | 1:5,000 | San Diego, CA | |
| Alexa fluor 647 | Mouse spleen dendritic cells | 1:200 | eBioscience, | 51-0114 |
| Armenian | | (FACS) | San Diego, CA | |
| hamster anti- | | | | |
| CD11c (integrin | | | | |
| alphax, p150/90) | | | | |
| Armenian | Mouse spleen dendritic cells | 1:100 | Pierce, | MA11C5 |
| hamster anti- | | | Rockford, IL | |
| CD11c MAb | | | | |
| (p150/95 chain) | | | | |

### Fluorescence Immunocytochemistry

Fixed sections were rinsed four times in 0.1 M TBS (Tris-buffered saline), followed by incubation in a TBS blocking solution containing 0.3% Triton X-100 in 0.5% BSA (Sigma, St. Louis, Mo.) at room temperature for 1 hour. Triton X was omitted when antibodies for surface molecules were used. Sections were washed and incubated in 0.1 M TBS containing primary antibodies at the dilutions listed in Table 1, or in BSA alone, as a control for nonspecific binding of secondary antibodies. Intra-assay controls included tissue incubated with secondary antibody alone (that is, no primary antibody). After overnight incubation at 4° C., sections were rinsed four times for 10 minutes each in 0.1 M TBS containing 0.1% BSA. Sections were incubated at room temperature for 1 hour in buffer containing 0.1% BSA in 0.1 M TBS and secondary fluorescence-tagged antibodies. All fluorescence-tagged secondary antibodies had an emission spectrum greater than approximately 600 nm to prevent overlap artifact with the EYFP signal. Secondary antibodies used were Alexa Fluor 633 goat anti-rat (1:1000; Molecular Probes, Carlsbad, Calif.; A-21094), Alexa Fluor 647 goat anti-hamster (1:1000; Molecular Probes; A-24451), Cy5 goat anti-rabbit (1:400; Jackson Immunoresearch, West Grove, Pa.; 111-175-003), and Cy5 goat anti-mouse (1:400; Jackson Immunoresearch; 115-175-003).

After incubation with secondary antibody, sections were rinsed four times for 10 minutes each in 0.1 M TBS containing 0.1% BSA and then rinsed one time 0.1 M PB, mounted on slides, and coverslipped with Aqua-Polymount (Polysciences). A Zeiss Axioplan-2 confocal laser scanning microscope was used to determine colocalization of the fluorescence-tagged antibody with EYFP+ bDC. One-micrometer serial Z-stack digital photographs were captured with LSM 510 software and were analyzed and collapsed into single images in Image J (Rasband, 1997-2004). Sections were also rotated in the orthogonal plane to confirm double labeling. Photomicrographs were collected and assembled from digital images for which colors were assigned and levels, contrast, and brightness were adjusted to optimize the images in Adobe Photoshop 7.0.

### Immunocytochemistry and Brain Map Generation

YFP is a variant of GFP, and the antibody for GFP (Table 1) reacts equally well with GFP and YFP proteins (Invitrogen Spec. Doc for GFP antibodies). Antibody to GFP was used to create distribution brain maps of EYFP+ bDC in the central nervous system (CNS). Free-floating fixed coronal sections of 7-week-old male mouse Itgax (CD11c)-EYFP brains were processed for immunocytochemical localization of EYFP according to the avidin-biotin complex (ABC) method (Hsu et al., 1981). Sections were incubated in 1) 0.5% BSA in 0.1 M Tris-saline (pH 7.6; TS) for 30 minutes; 2) chicken anti-EGFP antiserum (1:10,000) in 0.1% BSA in TS (or in diluent for controls) overnight at 4° C.; 3) biotinylated goat anti-chicken IgG (1:800; Vector Laboratories) in 0.1% BSA for 30 minutes; 4) peroxidase-avidin complex for 30 minutes; and 5) diaminobenzidine and H₂O₂ for 6 minutes (DAB peroxidase substrate kit; Vector Laboratories). Sections were mounted on 1% gelatin-coated slides, air-dried, dehydrated, coverslipped with DPX mounting medium (Aldrich, Milwaukee, Wis.), and photographed with a Nikon Optiphot Microscope with a Coolpix Digital camera. Brain sections were analyzed for the presence of EYFP+ bDC at 240 μm intervals (14-18 sections per animal). Photomicrographs were collected and assembled from digital images for which levels, contrast, and brightness were adjusted to optimize the images in Adobe Photoshop 7.0. Distribution diagrams of EYFP+ bDC were generated using Atlas Navigator (2000) and Adobe Illustrator 10 (Adobe systems) for regions of interest based on prevalence of stained EYFP+ bDC. Such regions were defined by clear landmarks using both Atlas Navigator and The Mouse Brain in Stereotaxic Coordinates (Franklin and Paxinos, 1997). The distribution of the marks in FIG. 6A-M represents an estimate of the number of cells seen per 40 μm section and are not intended to represent an exact number or a complete number of cells for an entire brain region.

### Electron Microscopy

Acrolein-fixed sections were treated with 1% sodium borohydride in PB (to remove active aldehydes) for 30 minutes prior to immunocytochemical labeling. Tissue sections then were rinsed in PB followed by TBS (pH 7.6) and incubated for 30 minutes in 1% BSA in TBS to minimize nonspecific labeling. Free-floating sections were processed for immunocytochemical localization of EYFP and Dcx using a pre-embedding peroxidase and immunogold-silver dual-labeling methods (Chan et al., 1990). Tissue sections were incubated in a cocktail containing chicken anti-GFP antiserum [1:3,000 (gold); 1:5,000 (peroxidase)] and goat anti-Dcx antiserum [1:2,000 (gold); 1:4,000 (peroxidase)] in 0.1% BSA in TBS for 24 hours at room temperature, followed by an additional 24 hours at 4° C. Immunoperoxidase labeling of EYFP was visualized using the ABC method. Sections were then incubated in 1) a 1:400 dilution of biotinylated IgG (anti-chicken for EYFP; anti-goat for Dcx) in BSA/TBS for 30 minutes; 2) peroxidase-avidin complex (at twice the recommended dilution; Vector) for 30 minutes; and 3) 3,3-diaminobenzidine (DAB; Aldrich) and H₂O₂ in TBS for 6 minutes.

Immunogold-silver detection of the anti-GFP antibody and/or anti-Dcx antibody was performed on tissue sections incubated in a 1:50 dilution of IgG conjugated to 1 nm colloidal gold particles (anti-goat IgG for Dcx; anti-chicken IgG for GFP; EMS, Fort Washington, Pa.) in 0.001% gelatin and 0.08% BSA in 0.1 M PBS for 2 hours at room temperature. Sections were rinsed in PBS, postfixed for 10 minutes in 1.25% glutaraldehyde in PBS, and rinsed in PBS followed by 0.2 M sodium citrate (pH 7.4). Conjugated gold particles were enhanced by incubation in silver solution (IntenSE; Amersham, Arlington Heights, Ill.) for 5-6 minutes. Dual-labeled sections were fixed for 60 minutes in 2% osmium tetroxide, dehydrated through a graded series of ethanols and propylene oxide, and embedded in EMBed 812 (EMS) between two sheets of Aclar plastic. Ultrathin sections (65-70 nm thick) through the CA1 region and dentate gyrus of a midseptotemporal level of the hippocampal formation were cut on a Leica UCT ultramicrotome. Sections then were counterstained with Reynold's lead citrate and uranyl acetate and examined with a Tecnai Biotwin electron microscope (FEI Company) equipped with an AMT digital camera. Photomicrographs were generated and assembled from digital images from which levels, contrast, and brightness were adjusted in Adobe Photoshop 7.0. Dcx- and EYFP-immunolabeled profiles were classified according to the nomenclature of Peters et al. (1991). Somata were identified by the presence of a nucleus. Dendrites contained regular microtubule arrays and mitochondria and were usually postsynaptic to axon terminal profiles.

FACS Analysis of EYFP+ bDC Cultures Derived from PN2 Brains

EYFP+ bDC cultures were prepared following standard protocols (Hassan et al., 1991; Sierra et al., 2007). PN2 Itgax (CD11c)-EYFP mouse pup brains were dissected on ice, and meninges were carefully removed under a dissecting scope. Forebrains were minced in 5% FCS-PBS, dissociated using fire-polished Pasteur pipettes, and then passed through a 40 μm nylon cell strainer (BD Biosciences, San Jose, Calif.). Cells were washed once in buffer and seeded in culture media (10% FCS Dulbecco's modified Eagle's medium (DMEM; Gibco, Carlsbad, Calif.)) at a density of approximately two forebrains per 75 mm flask. Culture medium was changed every 5 days and supplemented with 5 ng/ml granulocyte-monocyte colony-stimulating factor (GM-CSF; Sigma). After 10-14 days in culture, cells were shaken at 125 rpm for 5 hours at 37° C. Detached cells were harvested and counted by Trypan blue exclusion and equal numbers of cells distributed in a conical-bottom 96-well plate for immunostaining and FACS analysis.

CD11c Staining of EYFP+ cultures for FACS Analysis

Cultured cells were blocked in 5% mouse serum diluted in PBS for 15 minutes at 4° C. Cells were then incubated with 1 μg/mL of CD11c-Alexa Fluor 647 antibody (clone N418; eBioscience) or with 1 μg/mL isotype-hamster IgG-APC (clone eBio299Arm) for controls, for 15 minutes at 4° C. The cells were then washed twice in 2% FCS-PBS buffer and analyzed for CD11c by FACS with a FACS Calibur-2 (BD Biosciences) using FL1-H for YFP detection and FL4-H for Alexa 647.

Identification of CD11c mRNA in EYFP+ bDC

Viable EYFP+ bDC and EYFP− cells were harvested from adult brains using protocols developed for the isolation of EGFP+ microglia via FACS (Sierra et al., 2007). Adult mice were anesthetized with pentobarbital (750 mg/kg) and rapidly decapitated. Brains were quickly removed and placed on ice in Hank's balanced salt solution (HBSS; Gibco), and meninges, blood vessels, and choroid plexus were carefully removed under a dissecting scope. Brains were then placed in fresh HBSS, minced, and incubated with type 1′-S collagenase (600 U; Sigma) and DNAse (450 U; Invitrogen, Carlsbad, Calif.) for 30 minutes at 37° C. in 15 mL HBSS supplemented with 90 mM CaCl₂. After incubation, the cell suspension was further refined by repetitive gentle pipetting with fire-polished Pasteur pipettes on ice, followed by filtering through a 40 μm cell strainer (BD, Franklin Lakes, N.J.). Cells were then pelleted by centrifugation in a Sorvall RC-3B centrifuge (2,500 rpm, 5 minutes, 4° C.), resuspended in 10 mL of a 70% gradient buffer (as described below) and subjected to a gradient enrichment to increase further the yield of viable brain cells.

### Gradient Buffer

A stock solution of SIP (Amersham Biosciences, Upsala, Sweden) composed of Percoll and 10× PBS (9:1) was freshly made. Further dilutions of the SIP for the gradient were made with 1×PBS.

### Gradient Construction

To make the gradient, 2.5 mL PBS was placed in a 50 mL round-bottom polypropylene tube. Then, with a 10 mL syringe (without plunger) affixed to a 16 g needle, 7.5 mL of a 30% SIP solution was gently layered under the PBS, followed by 10 mL of the 70% SIP solution containing the resuspended cells. Gradients were centrifuged without break in a Sorvall RC5C Ultracentrifuge (1,200×g, 20 minutes, 20° C.). Cells were collected from the 30/70 interphase, washed with 5% FCS (Sigma)-PBS, pelleted, and resuspended in 5% FCS-PBS containing 100 ng/mL propidium iodide before sorting in a FACS Vantage SE Flow Cytometer (BD) with smHighPurity precision. Post sort analysis was performed to ensure the purity of the collection process.

### Real-Time (RT) PCR and Gene Array

FACS-sorted EYFP+ bDC and EYFP− cells were separately pelleted in 0.1 M PBS, and the supernatant was removed. Pelleted cells were then snap frozen in liquid nitrogen and kept at −80° C. until processing. RNA extractions were performed on the frozen EYFP+ and EYFP− FACS brain cells at SuperArray Bioscience Corp. (Frederick, Md.).

Reverse Transcription and First-Strand cDNA Synthesis

RNA samples were reverse transcribed (50 ng per sample) using a first-strand synthesis kit, Superarray RT2 PCR (C-02). This kit uses PowerScript reverse transcriptase and a combination of random primers and oligo-dT primers. The total volume of the reaction was 20 μL. The resulting cDNA was used to detect Itgax gene expression on Agilent Mouse Gene Array (SuperArray Bioscience Corp, Santa Clara, Calif.) and in RT-PCR. Agilent genechip arrays and analysis of the data with GenSpring software were performed at SuperArray Bioscience.

### RT-PCR

Real-time PCRs were performed using RT2 qPCR Primer Assay for Mouse Itgax gene (catalogue No. PPM03624E) and GAPDH (catalogue No. PPM02946E) primers on the Bio-Rad iQcycler using RT2 Real-Time SYBR green PCR master mix (PA-011). The total volume of the PCR was 25 μL. Thermocycler parameters were 95° C. for 10 minutes, followed by 40 cycles of 95° C. for 15 seconds and 60° C. for 1 minute. Relative changes in gene expression were calculated using the 2-Ct (threshold cycle) method (Livak and Schmittgen, 2001).

### Results

Initial analyses of brains from Itgax (CD11c)-EYFP transgenic mice revealed the presence of many EYFP+ bDC within discrete regions of the CNS. Fluorescent immunocytochemistry and subsequent confocal microscopy were performed with a panel of antibodies (see Table 1) against brain and immune markers of adult mice to determine the identity and lineage of these cells.

EYFP+ bDC Express Three Different Morphologies.

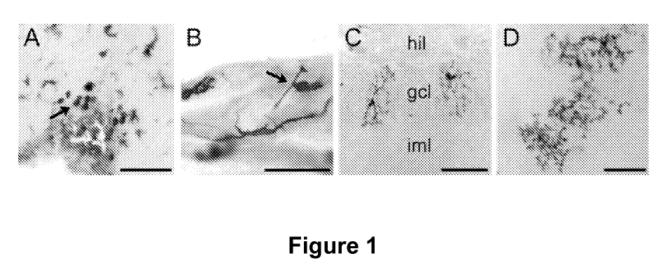

EYFP+ bDC were stained with antibodies against GFP and visualized using the ABC peroxidase method. Staining revealed three different morphologies of EYFP+ bDC in the CNS: 1) ovoid, 2) elongated/bipolar-like, and 3) stellate/dendriform (FIG. 1A-D). In the developing and neonatal brain, the predominant morphologies expressed by the EYFP+ bDC were ovoid and bipolar-like. These forms were also evident in the adult mouse along the ventricles and choroid plexus and within the circumventricular organs, such as the area postrema. Bipolar EYFP+ bDC were also oriented along the long axis of the nerves that traverse the adult CNS. Some elongated/bipolar-like EYFP+ bDC were also observed in transition zones between nerve fiber tracts and their nuclei of origin such as the point that the anterior commisure extends into the interstitial nucleus of the posterior limb of the anterior commissure (IPAC).

The third, and most common, EYFP+ bDC morphology noted in the adult CNS was the stellate/dendriform morphology. This form varied in shape from a more star-like (stellate) to more asymmetrical, tree-like (dendriform) morphology. The dendriform EYFP+ bDC were evident in the hippocampus, where their processes extended throughout the full width of the granule cell layer of the dentate gyrus (FIG. 1C). A predominance of stellate EYFP+ bDC were observed in layer II of the piriform cortex (FIG. 1D) and were also prevalent in other discrete regions of the parenchyma of the adult mouse brain, as described below.

### Confocal Analysis of Cell Phenotype

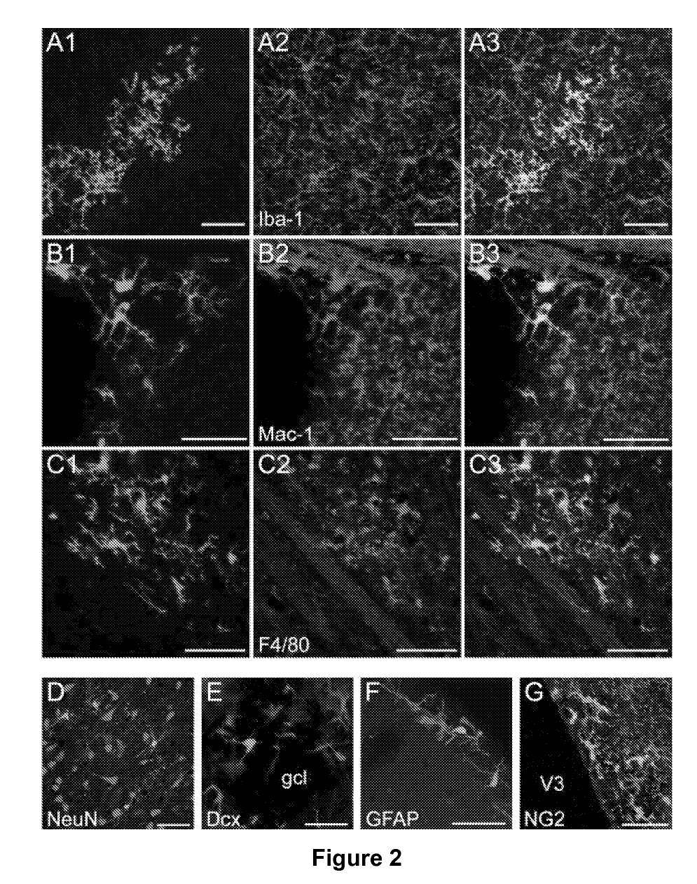

Characterization of the EYFP+ bDC in young adult males was performed using confocal fluorescence immunocytochemistry with well-characterized antibodies to specific brain and immunological markers, as shown in Table 1. Confocal Z-stack analysis and rotation of the stacks in multiple planes revealed that the EYFP+ bDC colabeled with a small fraction of the microglia identified with fluorescent-tagged antibodies against the immune markers Mac-1, Iba-1, and F4/80 (Table 1) as illustrated in FIG. 2A-C. All of the EYFP+ bDC examined also colabeled with antibodies against CD45, indicative of their bone marrow origin (data not shown). EYFP+ bDC did not colabel with the mature neuronal marker NeuN, the astroglial marker GFAP, the newly-born neuronal marker Dcx, or the oligodendrocyte/glia marker proteoglycan NG2 (FIG. 2D-G).

### Ultrastructural Analysis of EYFP+ bDC

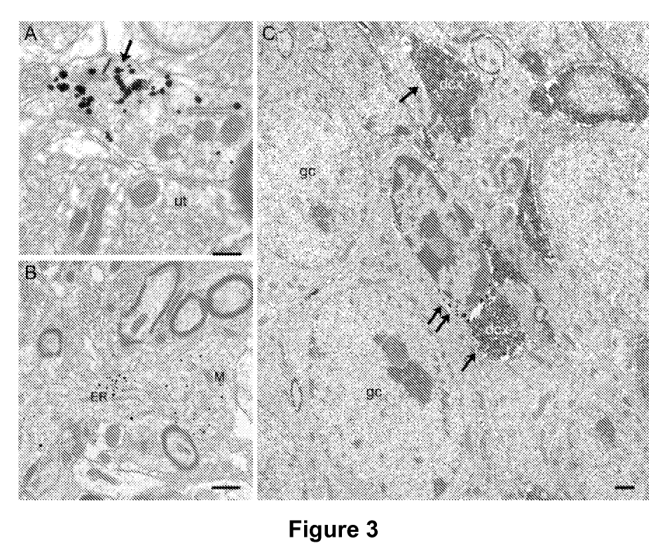

To characterize the EYFP+ bDC further, ultrastructural analysis was performed with antibodies against GFP and/or Dcx using the ABC peroxidase or gold-labeling methods. Two areas were examined: the hippocampal dentate gyrus, where asymmetrical, dendriform EYFP+ bDC reside, and the striatum, where stellate EYFP+ bDC were evident. GFP-labeled EYFP+ bDC profiles were often in close proximity to synaptic terminals (FIG. 3A). The GFP-labeled profiles were branched within the neuropil and contained labeling associated with mitochondria and abundant rough endoplasmic reticulum (FIG. 3B), similar to the ultrastructural morphology of microglia as described by Peters et al. (1991). GFP-labeled somata in the granule cell layer of the dentate gyrus had elongated nuclei with clumps of chromatin beneath the nuclear envelope and throughout the nucleoplasm, resembling monocytes. These properties have been attributed to both microglia in the CNS and DC in the periphery (Peters et al., 1991; El-Nefiawy et al., 2005). Labeled processes and cell bodies were observed in close apposition to Dcx peroxidase-labeled profiles in young postmitotic neurons (FIG. 3C).

### Verification of the Integrity of The CD11c Transgene

Colocalization of CD11c Protein in EYFP+ bDC: FACS Analysis Cultures

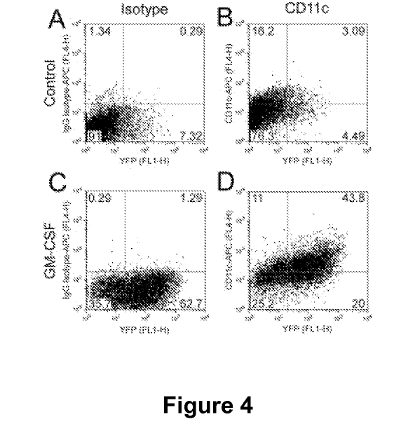

Recent studies indicate that among the postnatal heterogeneous microglia is a population of predendritic cells that can be driven toward a DC phenotype in vitro, in the presence of GM-CSF (Santambrogio et al., 2001). To determine whether EYFP+ bDC proliferated in response to GM-CSF and could be driven to express detectable levels of CD11c protein, we cultured PN2 brain preparations in the presence or absence of GM-CSF. Results of the FACS analysis showed that EYFP+ bDC were responsive to GM-CSF (FIG. 4). Control EYFP+ bDC cultured without GM-CSF accounted for only 7% of the total viable cells (FIG. 4A), whereas approximately 63% of the total viable cells were EYFP+ in the GM-CSF cultures (FIG. 4C). Among the EYFP+ bDC, 44% expressed both EYFP and CD11c antigen (FIG. 4D). A small population of EYFP+ bDC exhibited CD11c staining that did not change with GM-CSF treatment, possibly reflecting the presence of photobleached cells. FIGS. 4A and 4C represent isotype antibody controls for the CD11c (N418 antibody).

Colocalization of mRNA for Itgax (CD11c) in EYFP+ bDC Isolated from Adult Brains

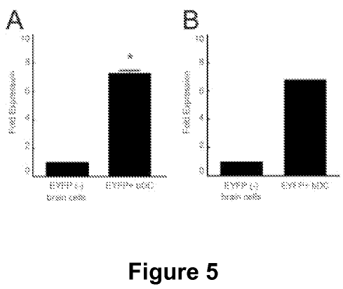

Three independent gene arrays were carried out on EYFP+ bDC and EYFP− cells isolated from adult brains of Itgax (CD11c)-EYFP transgenic mice by FACS. As illustrated in FIG. 5A, EYFP+ bDC expressed Itgax mRNA sevenfold over the EYFP− viable cells. This difference was further verified by RT-PCR (FIG. 5B).

Identification of EYFP+ bDC in Another CD11c Transgenic Mouse

To confirm further the integrity of the transgene construct, the inventors examined a different transgenic mouse that uses a CD11c promoter to drive the expression of GFP and the human diphtheria toxin receptor (Jung et al., 2002). Although these mice produce a weaker GFP signal, it was possible to demonstrate clearly by immunocytochemistry that the GFP-positive brain cells were similar in morphology and distribution to the EYFP+ bDC (data not shown).

### Discussion

### Morphological and Immunocytochemical Phenotype

EYFP+ bDC displayed three different morphologies (ovoid, elongated/bipolar-like, and stellate/dendriform). Although these morphologies could represent entirely different cell types, it is more likely that they represent a continuum of the same cell lineage in different developmental, positional, and functional modes. The ovoid form would be the most efficient for traveling through fluids, and an elongated/bipolar-like shape may be useful for attaching to and moving along fibrous tissue such as nerve tracts. In contrast, the highly ramified, stellate EYFP+ bDC were positioned within brain parenchyma in regions that receive molecules/antigens that bypass the BBB, via pathways such as the nasal-trigeminal-associated extracellular pathways identified by Thorne et al. (2004) and the circumventricular organs (Ganong, 2000). Dendriform variations of the EYFP+ bDC were also evident in regions that undergo dynamic neuronal plasticity in the adult, such as neurogenesis and synaptic turnover. The morphological diversity of an EYFP+ bDC continuum is further supported by real-time two-photon laser microscopy of live peripheral EYFP+ dendritic cells (Lindquist et al., 2004) and by the inventors' in vitro cultures of live EYFP+ bDC.

The fluorescent confocal microscopy findings confirm the monocytic lineage of EYFP+ bDC and their colocalization with a small fraction of microglia in both Z-stack and multiple-planes analysis. They were further distinguished by these methods from neurons, astrocytes, oligodendrocytes, and their precursors as well as from adult developing postmitotic neurons.

The identification of CD11c protein in the young, steady-state brains with the available antibodies has proved unreliable and inconsistent by confocal microscopy. Although it is possible that only the message for Itgax is expressed in the EYFP+ bDC, it is more likely that the CD11c protein is expressed in such low amounts in the steady state that it is below the level of detection by the CD11c antibodies. This hypothesis is supported by observations that many splenic EYFP+ cells are CD11c+ in the steady state using the same antibodies (Lindquist et al., 2004). Alternatively, the integrin CD11c protein may be associating with molecules other than the CD18 and thus would not be recognized by CD11c antibodies that detect the CD11c/CD18 heterodimer. Only precise molecular characterization of CD11c in the brain will resolve these issues.

### Example 2

### Neuroanatomic Distribution of Brain Dendritic Cells

### Results

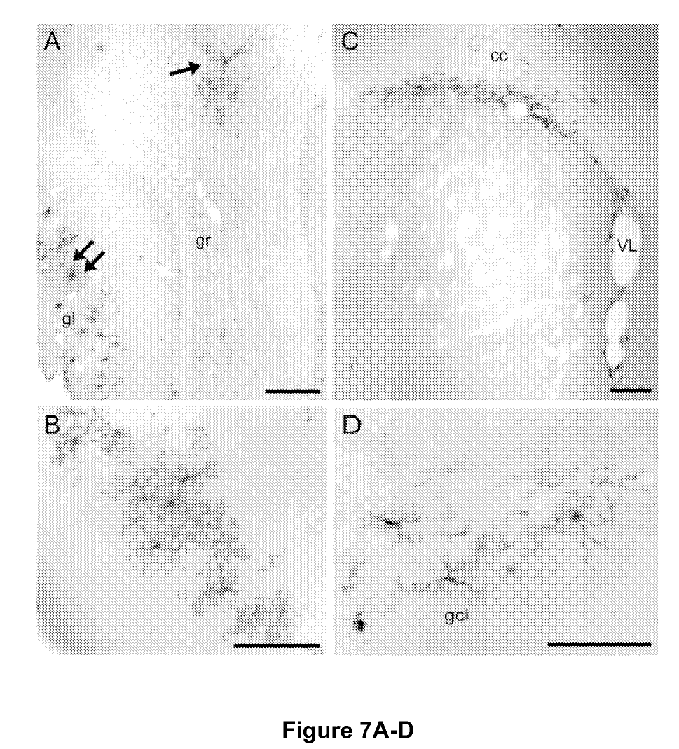

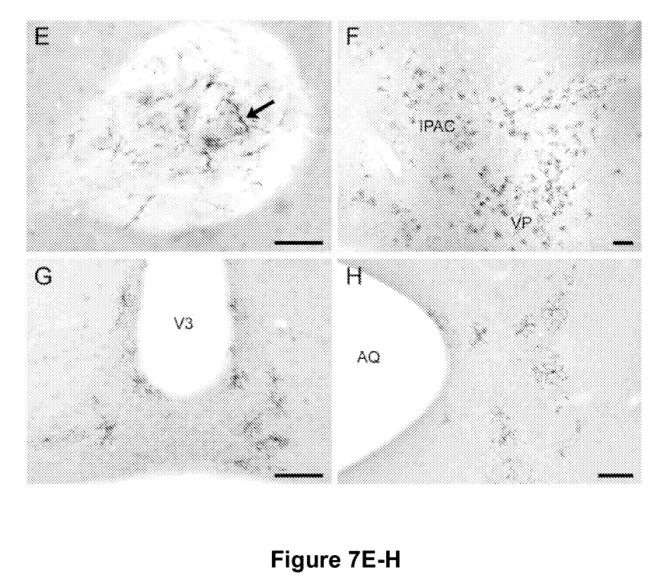

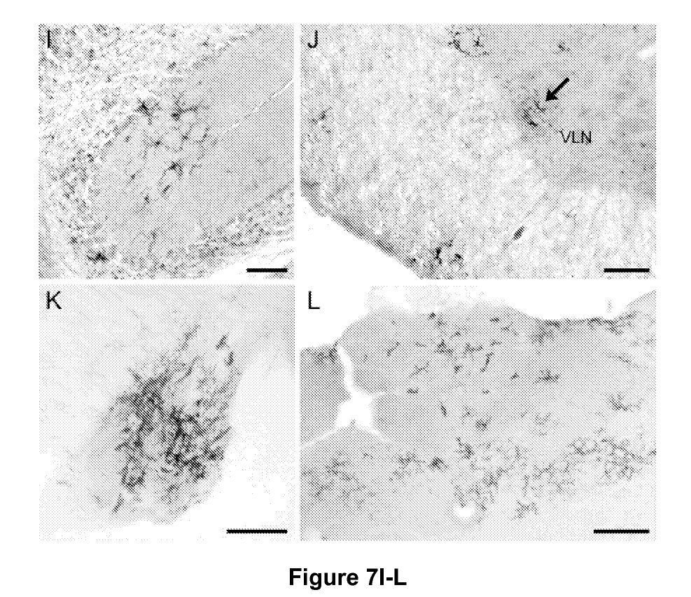

To gain insight into the possible functions of the EYFP+ bDC, the inventors mapped the anatomic distribution of EYFP+ bDC in the CNS in embryonic, neonatal, and adult mice using antibodies against GFP and the ABC peroxidase method. The distribution of the marks in FIG. 6A-M represents an estimate of the average number of EYFP+ bDC per 40 μm (approximately two cells per mark). Photomicrographs in FIG. 7A-L exemplify EYFP+ bDC distribution in several brain regions.

### Rostral Migratory Stream

Stellate EYFP+ bDC were present in the olfactory lobes on the border of the anterior commissure and surrounding the olfactory ventricle, a region often referred to as the subependymal layer (FIGS. 6A,B). In the adult, this region surrounds the anterior terminal end of the rostral migratory stream, a highway for newborn neurons traveling from the subventricular zone to the olfactory bulb (Nacher et al., 2001). EYFP+ bDC also lined the lateral, ventral, and ventromedial edges of the rostral olfactory bulb (for a discussion on plasticity, structure and function of the olfactory bulb and rostral migratory stream, see, for example, Peretto et al., 1999; FIGS. 6A-C, 7A). Additionally, EYFP+ bDC were noted within the olfactory nerve and the glomerular layer of the olfactory bulb and within the lateral olfactory tract (FIGS. 6A, 7A). EYFP+ bDC that were within the nerve tracts expressed a more elongated and less ramified morphology than the stellate cells observed in the parenchyma.

### Telencephalon: Cerebral Cortex, Including the Piriform Cortex

One of the most consistent distributions of EYFP+ bDC was noted within layer II of the piriform cortex. A high density of stellate EYFP+ bDC could be seen throughout this formation (FIGS. 6C-G, 7B). In the cerebral cortex, EYFP+ bDC were rare, appearing most often in layers II/III of the cortex. In the anterior prefrontal cortex (FIG. 6B), EYFP+ bDC were occasionally found throughout the prelimbic and medial orbital cortices. EYFP+ bDC also were present in the anterior cingulate, prelimbic, and infralimbic regions (FIGS. 6C,D). A few EYFP+ bDC were randomly distributed within the claustrum and dorsal endopiriform nucleus as well as occasionally in the posterior parietal and auditory cortices (FIG. 6F). Posterior to these regions, stellate EYFP+ bDCs were observed randomly throughout all of the layers of the entorhinal, ectorhinal, perirhinal, and lateral entorhinal cortices (FIG. 6G-I).

### Subventricular Zone, Corpus Callosum and Fiber Tracts

The forebrain exhibited unique concentrations of EYFP+ bDC in regions where postnatal neurogenesis occurs (Altman, 1969). One such area, along the lateral ventricles and adjacent septal tissue (FIGS. 6D,E), showed a distinct population of EYFP+ bDC similar to the macrophage-like cells described by Mercier et al. (2002). Here, EYFP+ bDC lined the dorsal and ventral edges of the lateral ventricles, within the subventricular zone. Where the lateral ventricle begins to extend and widen dorsally, these cells formed a linear array extending from the lateral ventricle through the forceps minor and genu of the corpus callosum (FIG. 7C). Labeled EYFP+ bDC also lined the lateral edge of the lateral ventricle and spread through the corpus callosum, striatum, and external capsule.

At the commencement of the internal capsule, elongated EYFP+ bDC extended along the fiber tract (FIGS. 6E,F). EYFP+ bDC were also evident throughout the fiber tracts of the stria medullaris of the thalamus and the stria terminalis as well as within the optic chiasm.

Hippocampal formation, fimbria/fornix

EYFP+ bDC were evident at the midline of the dorsal fornix and in the septofimbrial nucleus. At the level of the hippocampus, there was a high concentration of EYFP+ bDC in the fiber tracts of the fimbria and in the tissue bordering the lateral ventricle. Within the adult dorsal hippocampal formation (FIGS. 6F-H), EYFP+ bDC expressed a unique asymmetrical, dendriform morphology that differed from the more densely distributed stellate EYFP+ bDC in layer II of the piriform cortex. This was particularly evident in the granule cell layer (gcl) of the dentate gyrus (see FIGS. 1C, 7D), where these cells extend their processes through the densely packed granule cells within an area defined by the hilus on one side and the inner molecular layer on the other. This orientation was found in both the dorsal and the ventral blades of the dentate gyrus. Occasionally, the stellate form of EYFP+ bDC was noted at the very dorsal edge of CA1, extending into striatum oriens, and within the pyramidal cell layer of CA1 and CA3 (FIGS. 6F,G). Stellate EYFP+ bDC were also found along the hippocampal fissure. The distribution of these cells in the posterior hippocampal formation (FIG. 6H) was similar to that in the anterior region. EYFP+ bDC also were evident within the presubiculum and subiculum.

Extended amygdala and Autonomic Processing Areas

As described above, EYFP+ bDC were evident in the rostrocaudal extent of the lateral ventricles (FIG. 6D). These cells appeared around the border of the ventricle and continued into the anterior commissure, nucleus accumbens, and bed nucleus stria terminalis. Elongated EYFP+ bDC lay parallel to the axis of the commissural fibers and were persistently detected throughout the anterior commissure (FIG. 7E). Ventrolaterally from the bed nucleus stria terminalis, EYFP+ bDC extended along the curvature delineated by the nuclei and fiber tracts of the ventral striatum, ending just prior to the amygdala (FIG. 6D). Ventral to the striatum, these cells persist in the region of the extended amygdala called the substantia innominata (for nomenclature see Franklin and Paxinos, 1997) but herein referred to as the interstitial nucleus of the posterior limb of the anterior commissure (IPAC; see Paxinos and Franklin, 2001). The IPAC and surrounding areas contained the most widespread concentrations of stellate EYFP+ bDC found within the brain (FIG. 7F).

Anterior to the hippocampal formation, EYFP+ bDC were noted at the lateral edges of the septum (FIG. 6E). A few EYFP+ bDC were consistently identified at the midline, in the lateral septum and medial preoptic area, and near the ventral border of the third ventricle. Although EYFP+ bDC were noted in the nuclei and fiber tracts connected to and from the amygdala, including the extended amygdala, it was striking that no EYFP+ bDC were observed in the major nuclei of the amygdala (FIG. 6F).

### Diencephalon: Thalamus and Hypothalamus

There were relatively few labeled cells in the midbrain compared with other regions. In the thalamus, occasionally EYFP+ bDC were found embedded in the parenchyma at the midline, in the paraventricular thalamic nucleus and reunions nucleus (FIGS. 6F,G). EYFP+ bDC lay adjacent to the third ventricle, extending into the hypothalamus, and outlining the boundaries of the dorsal medial and periventricular nuclei. Many stellate EYFP+ bDC were evident within the arcuate nucleus (FIG. 7G), the ventral premammillary nucleus, and the median eminence (FIGS. 6F,G). More posteriorly, EYFP+ bDC were predominantly found at the lateral edges of the midbrain, including the optic tract and cerebral peduncles (FIGS. 6G,H).

Metencephalon and myelencephalon: periaqueductal gray, colliculi, and brainstem

EYFP+ bDC were consistently evident in the periaqueductal gray (FIG. 7H) and medial longitudinal fasiculus and ventrally in the medial mammilary nucleus (FIG. 6H). Some EYFP+ bDC were noted in the substantia nigra, medial geniculate nucleus, and medial lemniscus fiber tract. Cells were also found along the edges and midline of the superior colliculus. Stellate EYFP+ bDC were observed throughout the lateral periaqueductal gray, and clusters of these cells were noted below the aqueduct in the dorsal raphe nuclei. EYFP+ bDC appeared to follow the contours of the medial longitudinal fasiculus and raphe, through the fiber tracts of the medial lemniscus and into the decussation of the superior cerebellar peduncle (FIG. 6I). In the median raphe nucleus, these cells were bilaterally distributed in a short band in the tectospinal tract, which borders the median and paramedian raphe nuclei. Laterally, EYFP+ bDC were prominent in the middle cerebellar peduncle and in several nuclei of the sensory trigeminal nerve tract (FIGS. 6I,J). A rich population of labeled cells was also present in the lateral fiber tracts on the edges of the metencephalon-mylencephalon border as well as in the anteroventral cochlear nucleus, the vestibulocochlear nerve root (8N), the trigeminal nerve root (S5), and the principal sensory trigeminal nucleus.

EYFP+ bDC lined the lateral edge of the posterior brainstem (FIG. 6J,K) and were evident along the borders of the inferior cerebellar peduncle, the spinal trigeminal nuclei tract, and the rubrospinal tract. Many cells surrounded the border of the fourth ventricle, circumscribing the locus ceruleus, the medial vestibular nucleus, the prepositus hypoglossal nucleus, and the tegmental nuclei. Clusters of EYFP+ bDC were present at the midline, where the medial longitudinal fasiculus borders the fourth ventricle, and within the pyramidal tracts and medial lemniscus. EYFP+ bDC were also abundant within the ventral cochlear nucleus, trapezoid body, oral nucleus of the spinal trigeminal tract, dorsal motor nucleus 10, fiber tracts of the facial nerve (7N), and facial nucleus. Dorsally, EYFP+ bDC were evident at the upper edges of the external cuneate nucleus, in the nuclei of the solitary tract, and heavily concentrated in the hypoglossal nucleus (N12). Ventrally, EYFP+ bDC were densely distributed along the curvature of the inferior olive nuclei.

### Cerebellum

In the cerebellum (FIGS. 6J,K, 7I), EYFP+ bDC were most evident in the granule cell layer and scattered within the white fiber tracts. EYFP+ bDC were also located near the deep nuclei and in the medial cerebellar peduncle, the inferior cerebellar peduncle, and the anterior interposed nucleus (FIG. 6K).

### Spinal Cord

In the cervical and thoracic spinal cord (FIGS. 6L,M), EYFP+ bDC were observed at the borders between the white and the gray matter (FIG. 7J). The majority of these cells were located in the dorsal horn, ventral horn and ventrolateral nuclei (FIG. 6M). Occasionally, these cells formed a secondary row along the outer edge of the ventral funiculus and along the ventral median fissure, as described by Sidman et al. (1971).

### Circumventricular Organs and Choroid Plexus

EYFP+ bDC were prominent within all of the circumventricular organs examined (area postrema, subformical organ, choroid plexus, median eminence, and pituitary); however, there was a marked difference in the morphology of these cells within many of these regions. As shown in FIG. 7K, the subformical organ contained primarily stellate cells within its boundaries, whereas the area postrema displayed a gradient of EYFP+ bDC shapes, from the more ovoid and bipolar forms near the ventricles to a stellate morphology within the transition zones into the brain (FIG. 7L).

### Example 3

### EYFP+ bDC Distribution and Marker Phenotype in Embryonic and Neonatal Brains

The experiments in this Example were directed toward understanding the distribution and phenotype of bDCs in developing brains, their lineage, and their origin. Detection of bDC in the early embryonic brain, for example, would strongly suggest that they are resident cells rather than cells that only infiltrate the brain during trauma and/or disease.

### Materials and Methods

### Transgenic Animals

CD11c-EFYP+ were obtained, bred, and maintained as described above.

### Perfusion and Tissue Storage for Immunohistochemistry

Animals were given a lethal dose of Nembutal (Sodium Pentobarbital, Henry Schein, 100 mg/kg) and were transcardially perfused initially with saline containing heparin (1 unit/mL) and subsequently with 4% paraformaldehyde in 0.1 M phosphate buffer. Brains were post fixed in 4% paraformaldehyde overnight and submerged in 30% sucrose. Embryonic tissue was collected after transcardial perfusion of the mother. Embryonic day 10, 16 and the postnatal day 0 tissues were frozen and cut on cryostat into 20 μm sagittal or coronal sections. Sections were collected on Vectabond (Vector Labs, Burlingame, Calif.) coated slides and were stored at −80° C. until ready to use. In addition to the stages studied above, PN2 mouse brains were studied in tissue culture experiments.

Tissues from PN1, PN8, PN15, and PN21 mice were frozen and cut into 40 μm thick coronal sections on a freezing microtome. Sections were stored at −20° C. in cryoprotectant (25% ethylene glycol, 25% glycerol in 0.1M PB, pH7.4) until ready for use for floating immunohistochemistry.

GFP (Green Fluorescent Protein) Immunohistochemistry with Avidin-Biotin Complex (ABC) Method to Localize the EYFP+ Cells in Embryonic and Early Postnatal Life

Sections were rinsed 4 times with 0.1 M TBS, incubated in 1) 0.5% BSA in 0.1 M Tris-saline (pH 7.6; TS) for 30 minutes; 2) chicken anti-EGFP antiserum (1:10,000) in 0.1% BSA in TS or in diluent, for controls, overnight at 4° C.; 3) biotinylated goat anti chicken IgG (1:800; Vector Laboratories) in 0.1% BSA for 30 minutes; 4) peroxidase avidin complex for 30 minutes; and 5) diaminobenzidineand H₂O₂ for 6 minutes (DAB peroxidase substrate kit; Vector Laboratories). Sections were mounted on 1% gelatin-coated slides, air dried, dehydrated, cover slipped with DPX mounting medium (Aldrich, Milwaukee, Wis.), and photographed with a Nikon Optiphot Microscope with a Coolpix Digital camera.

Fluorescence Immunohistochemistry to co Localize Markers on EYFP+ Cells in the CD11c/EYFP Transgenic Animals

Sections were rinsed 4 times with 0.1 M TBS, and were preincubated with 0.5% Bovine Serum Albumin (BSA, Sigma) at room temperature for 1 h. Primary antibodies were incubated at 4° C. in 0.1% Triton X-100, 0.1% BSA/0.1 M Tris-buffered Saline (TBS) overnight. After 4×10 minute rinses with 0.1% BSA in 0.1 M TBS, fluorochrome-conjugated secondary antibodies were incubated in the dark at room temperature for 1 h in 0.1% TritonX-100, 0.1% BSA/0.1M TBS. Far-red fluorochromes were used to avoid overlap with the EYFP signal. Rinses (4×10 min) were repeated in the dark. Sections were mounted in 0.1 M PB on slides, coverslipped with Aqua-Polymount (Polysciences, Warrington, Pa., USA) medium and stored at 4° C. in the dark. A series of Z-stack images of sections 1 μm apart was taken by a confocal laser scanning microscope (Zeiss Axioplan2) with LSM 510 software. EYFP+ cells were analyzed for double labeling with other markers.

### Results and Discussion

Because EYFP+ bDC examined were positive for immune-cell-lineage antibodies Mac-1, CD45, Iba-1, and F4/80, the inventors wanted to determine whether the distribution of the EYFP+ bDC in the developing and adult CNS was comparable to the distribution of F4/80-positive microglia, as described elsewhere (Hume et al., 1983, 1984; Perry et al., 1985). Thus, in addition to the adult male brains, several time points of brain development were also examined. E10 brains were examined because early neurogenesis occurs at this time. E16 brains were chosen because E16 is the time point when the blood-brain barrier is formed. Brains from postnatal days P0 and P7 were also examined, as they represent critical periods of brain development. Brain cells derived from PN2 animals, which are amenable to culturing, were also examined. Other stages that are being examined in ongoing experiments include PN15 and PN22, which also represent critical periods of brain development.

Results from brain mapping experiments in embryonic and neonatal brains are also expected to help identify the precursors of adult bDC in the developing brain, thereby possibly shedding insight into the lineage of bDC.

These experiments are ongoing, and results obtained thus far are discussed below.

### Embryonic

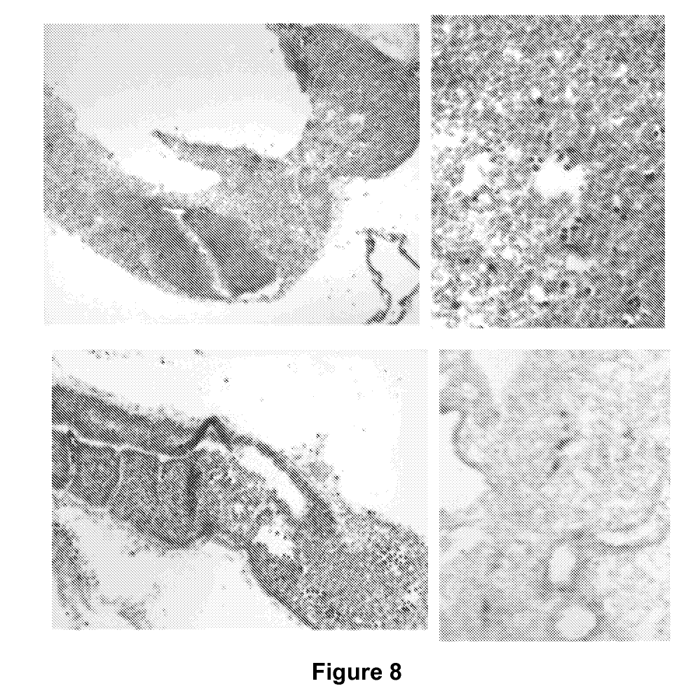

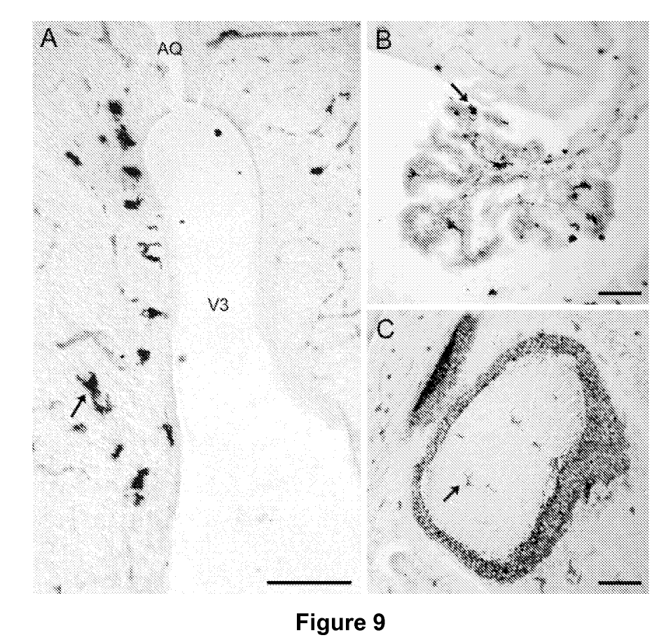

EYFP+ bDC were detectable as early as embryonic day 10 (FIG. 8). Ovoid and bipolar-like EYFP+ bDC were evident in E16 brains and in many organs throughout the entire body. In the developing brain, they were present along the ventricles and within the adjacent parenchyma (FIG. 9A), in the choroid plexus (FIG. 9B), and in and around the developing eye tissues (FIG. 9C).

### Neonatal

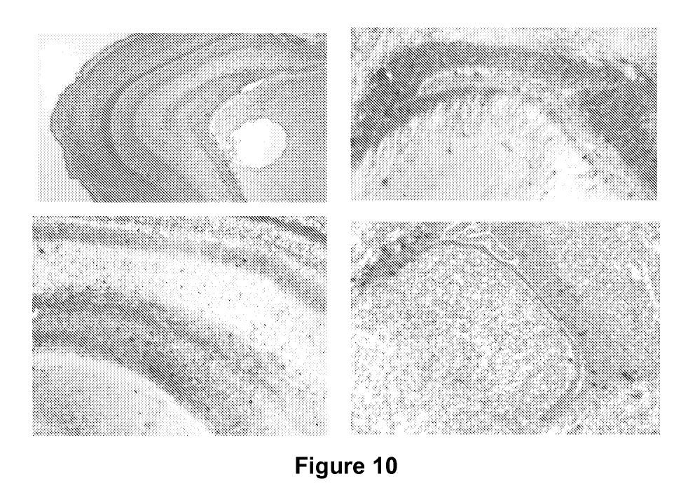

EYFP+ bDC were abundant in stages P0 and P7 within regions of perinatal neurogenesis (FIG. 10 and data not shown).

Analysis was also conducted on cultured cells from neonatal brain. Neonatal brain cells including neurons, astrocytes, and microglia can be grown and expanded in primary cultures. Among PN2 cells that were cultured, EYFP+ bDC were present immediately following the initial seeding (FIG. 11A). Fixed tissue from this age group revealed the presence of both ovoid cells and a slightly ramified/stellate form of EYFP+ bDC within the developing parenchyma (FIG. 11B). Ovoid and bipolar-like EYFP+ bDC were found lining the ventricles (FIG. 11C).

### Marker Expression

Immunostaining for a variety of markers was conducted to determine if certain distinguishing markers are expressed in bDC during different phases of development. Characterization of bDC marker phenotypes during embryonic and perinatal stages is expected to not only shed light on the lineage of bDC, but also facilitate methods of identifying such cells for diagnostic and therapeutic uses.

Microglia markers F4/80, Mac-1, and Iba-1 were found to colocalize with EYFP+ bDC at E16 and at P0 (data not shown). Preliminary data suggest that EYFP+ bDC in E10 embryos may also colocalize with microglia markers.

Other markers being examined include stem cell markers of a certain lineages. For example, CD105, CD9, and CD44 are transmembrane proteins that serve as early macrophage/microglia lineage markers whose expression can be examined by immunostaining with available antibodies. Expression of hematopoietic stem cell markers such as CD34, SLAM/CD150, CD31, CD41, PU.1 (dendritic cell lineage transcription factor), and MAfB (macrophage lineage transcription factor) can also be examined by immunostaining. Nestin and S100β are expressed in developing neurons and glia and may be used as markers of neurogenesis.

### Discussion

The embryonic brain was examined to determine whether EYFP+ bDC are present in the CNS once the BBB has formed. Further, the inventors wanted to address whether DC enter the adult brain following trauma or are derived from a resident population. Results showed that EYFP+ cells resembling monocytes were present in the developing brain as early as E10, thus supporting the notion of DC/microglia precursors residing in the adult brain. EYFP+ bDC were also present during the perinatal period and amenable to expansion in culture. The immune system is not functional in the mouse until the third postnatal week, so it is possible that EYFP+ bDC in the developing CNS take on functions involved in the organization of brain structures. These bDC could respond to signals from the milieu during this stage of development that induce such basic functions as phagocytosis and removal of cell debris. Likewise, EYFP+ bDC might maintain a developmental residual function in the adult CNS, as others have postulated for the microglia, and be involved in synaptic plasticity and neurogenesis over the life span of the mouse (Cohen et al., 1997; Cohen and Schwartz, 1999; Nair et al., 2004; Kipnis and Schwartz, 2005; Butovsky et al., 2005, 2006a,b; Schwartz et al., 2006; Schwartz and Yoles, 2006; Ziv et al., 2006a, b)

A possible function for brain DC in neurogenesis is reinforced in the present study by their location in regions well recognized for postnatal neurogenesis and neural remodeling, e.g., the dentate gyrus in the normal adult (Gould and Cameron, 1996) and following seizures (Jessberger et al., 2005), the subventricular zone of the lateral ventricles (Mercier et al., 2002), the olfactory pathways comprising the rostral migratory stream, and within layer II of the piriform cortex (Ramirez-Castillejo et al., 2002). Other studies have suggested a role for microglia in neurogenesis, but none has identified any unique features of these cells that distinguish them from the general microglia population. Perhaps the strongest evidence supporting a role for DC in neurogenesis comes from the experiments of Mikami and colleagues (2004) in adult mice. Their in vitro studies revealed that peripherally derived DC initiated the proliferation and survival of neural stem/progenitor cells, whereas their in vivo studies showed that damaged spinal cords demonstrated significant structural and functional recovery following the combined introduction of splenic DC and neural stem/progenitor cells into the injured area. These data suggest that among the DC is a subpopulation of cells that can initiate neurogenesis. What is not clear is whether the resident spinal cord brain DC are disinhibited by their splenic DC cousins and initiate the repair or whether the splenic DC themselves respond directly to CNS environmental cues and initiate repair and regeneration. The recent paper by Butovsky et al (2007) suggests that some DC are recruited to brain from bone marrow in the adult and are involved in brain damage, such as plaque formation. Nevertheless, Butovsky et al. does not address the function of a resident brain DC population in the steady state, which the inventors have established is already present in embryos at and before the time of the closure of the BBB.

It may be beneficial to view the distribution of EYFP+ bDC within other regions of the brain in light of their function as immune sentinels (Steinman et al., 1980). In young adult mice, numerous bipolar-like EYFP+ bDC were distributed in olfactory-related nerve bundles that traverse the brain, allowing access to their nuclei of origin and surrounding parenchyma. Such bipolar EYFP+ bDC were observed in the anterior commissure, where they were distributed in the nerve bundle, and a rich population of stellate EYFP+ bDC was dispersed throughout brain nuclei associated with these fibers, such as in nucleus accumbens, bed nucleus stria terminalis, IPAC, and ventral pallidium (Jouandet and Hartenstein, 1983).

There were also large populations of EYFP+ bDC associated with regions that lack a BBB, such as the circumventricular organs. These areas can allow direct access of pathogens and harmful chemicals from the CSF and blood into the parenchyma of the adjacent brain tissue. Clear examples of the presence of EYFP+ bDC in these regions were seen in the relationship of the hypothalamic arcuate nucleus to the median eminence and area postrema to the brainstem and cerebellum. Additionally, stellate EYFP+ bDC were distributed along the two extracellular pathways that bypass the BBB and provide rapid transport of molecules into the CNS (Thorne et al., 2004). Stellate EYFP+ bDC were clearly evident in the pathway associated with the peripheral olfactory system, connecting the nasal passages with the olfactory bulbs and rostral brain regions, as well as along the pathways associated with the peripheral trigeminal system, which connects the nasal passages with brainstem and spinal cord regions (Liu et al., 2001). Both routes are thought to be involved in the delivery of molecular signals directly into the brain, like those generated by pheromones and vasanas (McClintock et al., 2001), but they are also vulnerable to opportunistic invasion by many airborne chemicals and pathogens that could result in significant damage to the CNS. Thus, the distribution of the stellate EYFP+ bDC along these routes would be consistent with the immune activation and/or tolerance mechanisms carried out by DC in the periphery. This rationale is further supported by studies showing that DC injected into the brain travel via the CSF to the deep cervical lymph nodes to carry out their immune function (Pile-Spellman et al., 1984; Lowhagen et al., 1994; Walter et al., 2006; Hatterer et al., 2006).

Although data presented herein are highly suggestive of the presence of a population of specialized DC within the steady-state CNS, it is also known that under extreme circumstances, peripheral DC enter the brain and compromise or influence the role of the bDC. Given that EYFP+ bDC can now be distinguished among the heterogeneous population of cells collectively referred to as microglia, CD11c-EYFP+ mice are a valuable tool to determine the nature of the contributions of bDC to plasticity and neurogenesis in the adult steady state and to immune regulation in the pathological state.

### Example 4

### Distribution of bDC in Aged Mice

In the present Example, the distribution of bDC was examined in aged mice to elucidate possible roles for bDC in aging processes such as cognitive decline.

### Methods

CD11c-EYFP Transgenic Mice

Itgax (CD11c)-EYFP mice were obtained and bred as described above.

In this Example, male mice were used at 10 weeks (young adults) and at 22-24 months (aged). Mice were genotyped for the presence of the EYFP transgene, using RT-PCR methodologies.

### Perfusion and Tissue Collection

For light and fluorescent microscopy, mice were deeply anesthetized with sodium pentobarbital (150 mg/kg, i.p.) followed by sequential perfusion through the left ventricle with saline, containing heparin (1 unit/mL), and then with 4% paraformaldehyde (PFA) in 0.1 M phosphate buffer (PB). Brains were removed and immersed in the same fixative for 4 h at 4° C., and subsequently rinsed in PBS. Coronal sections (40 μm) were cut on a Leica vibratome (Microm, Walldorf, Germany), serially collected and stored at −20° C. in cryoprotectant (25% ethylene glycol, 25% glycerol in 0.1 M PB, pH 7.4).

### Immunostaining and Fluorescence Immunocytochemistry

Antibodies used are presented in Table 1 and are as described above. Immunostaining was carried out using DAB visualization; protocols for immunostaining and immunocytochemistry are as described above.

### Quantification and Stereological Counting Procedure

For stereological analysis of the DAB-stained sections 40 μm sections were examined using a Nikon (Eclipse E600) light microscope at 40× magnification and the program Stereo Investigator (version 07, Microbrightfield). The number of CD11c-EYFP cells was quantified by applying a set counting window over the region of interested in matched sections. Matched sections and regions of interest were identified using landmarks from the Franklin and Paxinos brain atlas referenced above.

EYFP+ cells were counted in certain areas of serial sections as discussed below. For the forceps of the corpus callosum 40 μm sections (6-8) were taken from Bregma −1.50 mm to −1.96 mm. Here an area (63,000 μm²) was chosen in both hemispheres within the corpus callosum closest to the subventricular zone, since the young animals showed the highest concentration of cells within these fiber tracts. For the entorhinal/ectorhinal/perirhinal cortex, 40 μm sections (6-8) were taken from Bregma −1.22 mm to −2.30 mm and an area of 240,000 μm was identified and analyzed in both hemispheres. For the prefrontal cortex 40 μm sections (6) were taken from Bregma −2.80 mm to −1.42 mm and EYFP+ cells counted in an area of 123,000 μm² in both hemispheres. The density was calculated by dividing the volume of the region of interest by the average number of cells, using a depth of 40 μm, averaged for both hemispheres. Densities were then calculated to the volume of one cubic millimeter.

For the cerebellum and the genu of the corpus callosum two sections per animal (40 μm) were matched using the brain atlas as a guide, and areas of 52,000 μm² were chosen to be counted. For the dentate gyrus, an area of 120,000 μm² including where the top and the bottom blade of the dentate gyrus join together was chosen in matched sections for both hemispheres. For these three areas, density was again calculated by dividing the volume of the region of interested by the average number of cells for each animal. Densities were then calculated to the volume of one cubic millimeter. Groups were defined as aged (>22 months) or young (10 weeks). Four male mice were analyzed for each group. Significance was analyzed by an unpaired Student's t-test for each region of interest (P<0.05).

For analysis of fluorescent sections and quantification of IBA-1 cells, the region above the subventricular zone within the forceps of the corpus callosum was again chosen. Two matched sections per animal were identified and the forceps of the corpus callosum of both hemispheres was photographed in an identical counting frame (52,000 μm²). A Zeiss Axioplan-2 confocal laser-scanning microscope was used to visualize and determine colocalization of the fluorescence-tagged antibody Iba-1 with EYFP+ bDC. One-micrometer serial Z-stack digital photographs (n=14) were captured with LSM 510 software and were analyzed and collapsed into single images in Image J (Rasband, 1997-2004). Sections were also rotated in the orthogonal plane to confirm double labeling. The collapsed pictures and were analyzed using Stereo Investigator (version 7). Statistics were examined by ANOVA and post-hoc test (Tukey-Kramer).

### Results

The immune phenotype of these cells were further characterized in young versus aged CNS. As in the young adult animals, the monocyte immune markers for F4/80, CD11b and Iba1 were present in all of the bDC examined in the aged animals.

Cultures of bDC have also demonstrated their capacity to make cytokines further demonstrating the functionality of these cells. An overall increase in the number of EYFP+ bDC was observed in aged animals as compared to young animals. In young animals, EYFP+ bDC were found scattered in distinct regions such as piriform cortex, dentate gyrus, and hypothalamus. In aged animals (24 months), a two to three fold increase in the number of EYFP+ bDC was detected within the fiber tracts, including the corpus callosum, fornix, fimbria, hippocampus and the septum (p=<0.05), as well as in. several cortex regions and the cerebellum (FIGS. 12A-B).

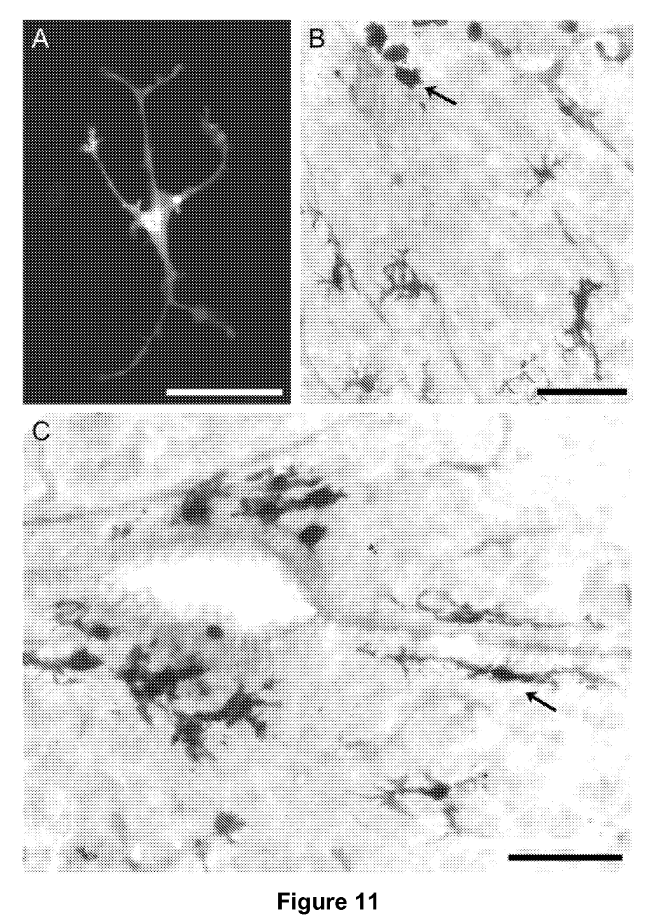

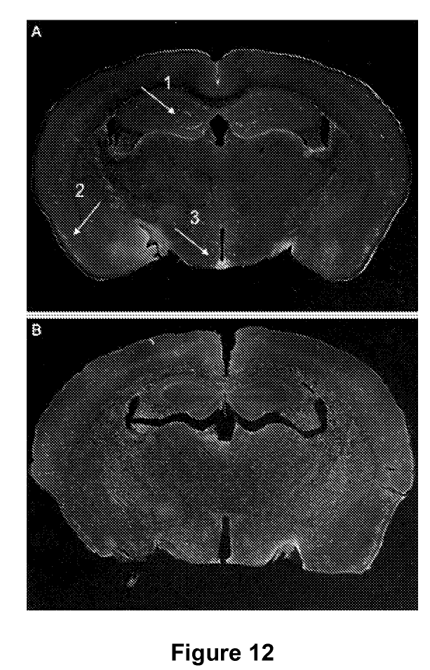

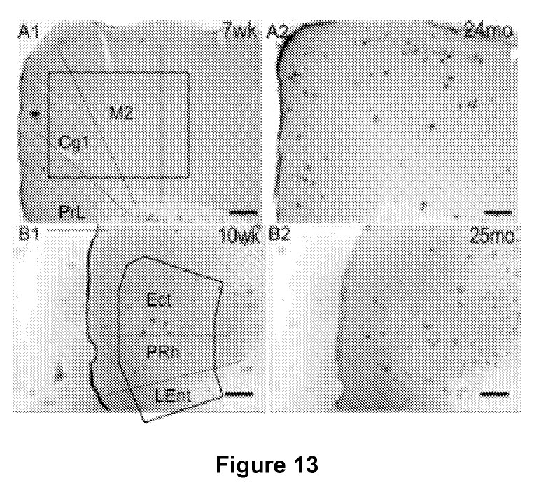

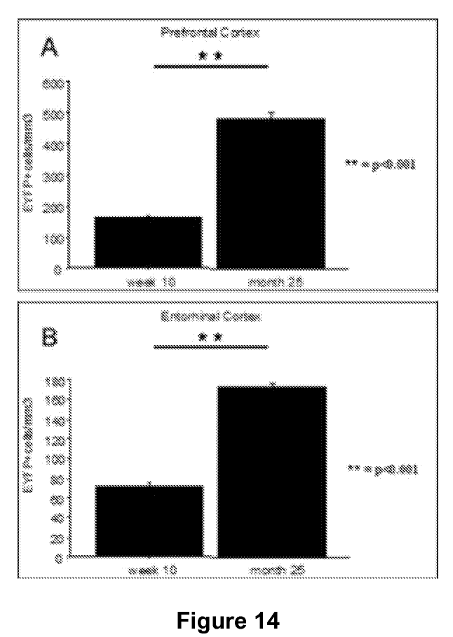

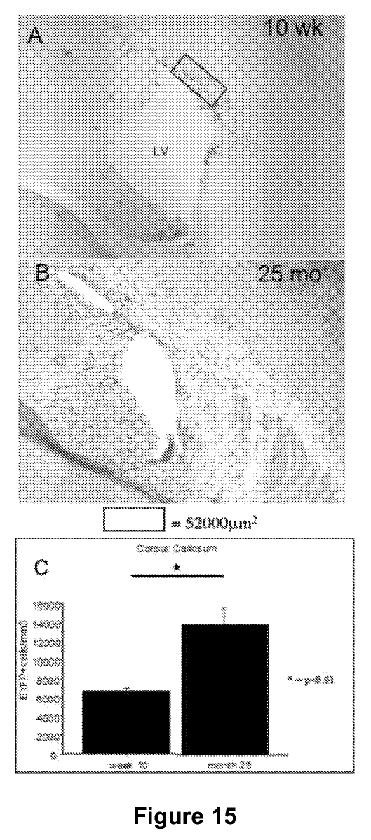

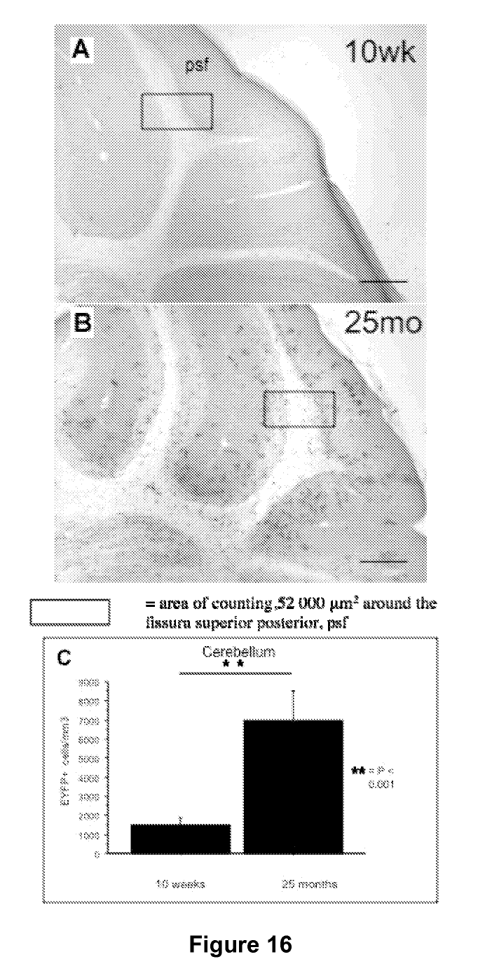

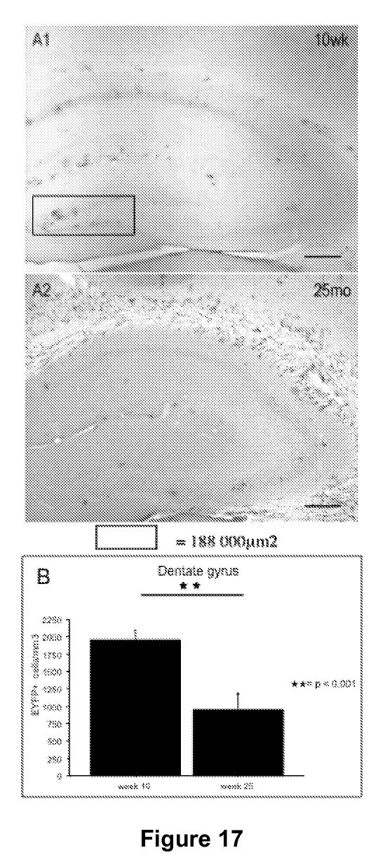

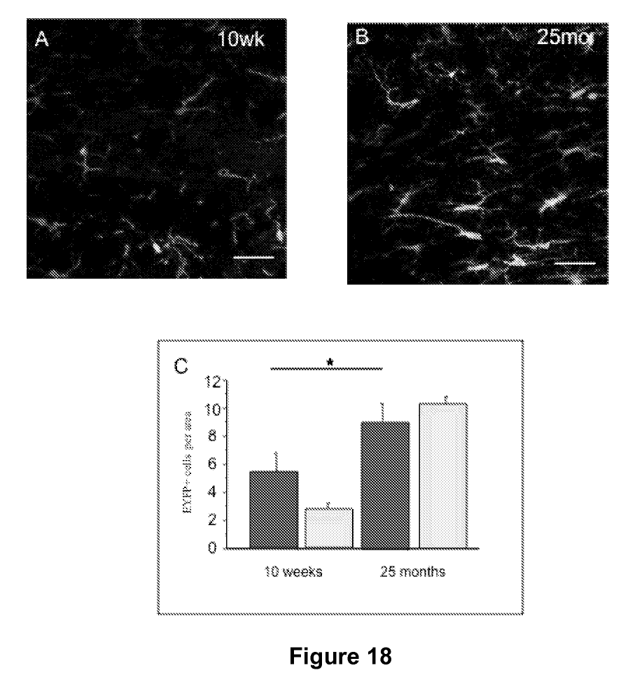

As shown in FIGS. 13-16 and 18A-B, increases of 2 to 4.5 fold higher in the number of EYFP+ cells were observed in aged mice in many areas. Such areas included brain regions implicated in age-associated cognitive decline such as prefrontal cortex, entorhinal cortex, corpus callosum, and cerebellum. In the corpus callosum, forceps area, the ratio of bDC versus microglia significantly increased in aged animals. On the other hand, a decrease in the number of bDC in the dentate gyrus of the hippocampus, an area associated with neurogenesis, was observed in aged animals (FIG. 17).

### Discussion

As the bDC represent a member of the dendritic cell family, these results suggest a role for the “natural” accumulation of these immuno-associated cells in old animals. Without wishing to be bound by any particular theory, it is proposed that such accumulation may be due to changes in the inflammatory environment during aging.

Accumulation of brain dendritic cells was visible in areas vulnerable to aging and/or involved in the onset of Alzheimer's Disease. Such accumulation may contribute to age-related disorders such as Alzheimer, Parkinson, and Huntington diseases.

### Example 5

### Activation of bDC Following Seizure

To investigate a possible role for bDC in neuronal injury and/or neurodegeneration, bDC were studied in a mouse seizure model.

### Methods

### Kainic Acid Treatment

Seizure induction, resulting in neuronal insult and neurodegeneration, consisted of a single i.p. injection of vehicle (saline) or 30 mg/kg kainic acid (Sierra et al., 2007), administered to male Itgax (CD11c)-EYFP(N=6) and male cfms (CSF-1R)-EGFP transgenic mice (N=3) 6-8 weeks of age. Only animals achieving stage 4 seizures (Lothman and Collins, 1981) were used. Control animals (N=3 per group) received i.p. saline diluent injections. Mice were anesthetized and perfused 48 hours after seizure induction, as described below.

Criteria used by Raivich et al. (1999) for the activation stages of microglia were adapted to evaluate the morphology of EYFP+ bDC and EGFP+ microglia in the damaged hippocampus 48 hours post seizure. Stage 0=steady state: highly ramified dendriform/stellate; stage 1=alert: thicker, less ramified processes; stage 2=homing: truncated cells aggregated around damaged neuronal zone; stage 3a=activation: spherical cells clustered around damaged tissue; stage 3b=bystander activation: cells in regions adjacent to damage expressing an alert morphology.

### Results

Evaluation of EYFP+ bDC following kainic acid lesions

A seizure model was used because of the investigators' past experience with the responses of microglia in the cfms (CSF-1R) EGFP transgenic mouse and because dendritic cells have been reported in the CNS following kainic-acid-induced seizures (Newman et al., 2005). Changes in morphology and distribution of the GFP+ microglia and EYFP+ bDC following seizures were assessed by using a modification of the four-stage criteria developed by Raivich et al. (1999; see Materials and Methods).

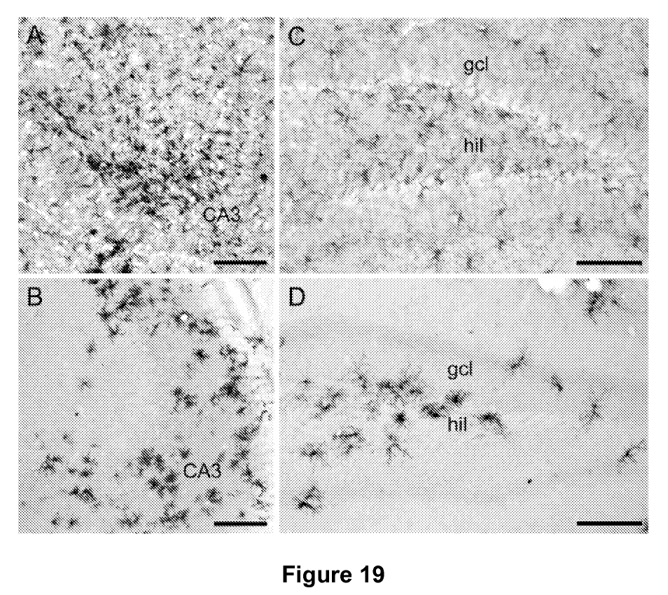

EGFP+ microglia in the cfms (CSF-1R)-EGFP and EYFP+ bDC in the Itgax (CD11c) EYFP mouse brains exhibited marked changes in both morphology and distribution within the CA3 region of the hippocampus 48 hours following seizure induction. EGFP+ microglia, which are uniformly distributed in the steady-state hippocampus of cfms (CSF-1R)-EGFP transgenic mice, displayed stage 3a activated morphology, as indicated by their spherical, hypertrophied cell bodies in and around the region of damage. EGFP+ microglia immediately outside the injury zone of CA3 also expressed a stage 3b bystander alert morphology (FIG. 19A). In the Itgax (CD11c)-EYFP mouse brain, a discrete population of EYFP+ bDC was noted clustered in and around the CA3 region of neuronal damage and displayed morphology corresponding to stages 2 and 3a (FIG. 19B). Directly outside the penumbra of the damaged CA3 neurons, EGFP+ microglia maintained distinct 3b alert bystander morphology (FIG. 19A), gradually shifting to a stage 1 and stage 0 morphology at points more distal to the lesion zone, such as the dentate gyrus (FIG. 19C). In contrast, the morphology and distribution of EYFP+ bDC distal to the CA3 zone of damage were markedly different from those described for the steady state. Stage 3b alert EYFP+ bDC were prominent in hilus of the dentate gyrus and subgranule layers (FIG. 19D), especially in regions where ectopic neurogenesis has been noted following seizures (Parent and Lowenstein, 2002)

### Example 6

### bDC Response and Calcitonin Gene-Related Peptide (CGRP)

Example 5 established that bDC are sensitive to KA administration. To further elucidate the possible role of bDC in neuronal injury following seizure, the relationship of bDC to calcitonin gene-related peptide (CGRP) was examined in the present Example.

CGRP is a neuropeptide released from sensory nerve fibers in the periphery that has immune modulatory functions. Interestingly, CGRP has been localized to fibers and some cell bodies in the rodent forebrain, and its expression has been demonstrated to upregulate in regions of damage following insults such as ischemia or seizure. For instance, the investigators have previously reported that CGRP expression is increased in mossy cells and in the mossy fiber terminals (MFT) of dentate granule cells in the hippocampus following kainic acid (KA)-induced seizure. KA-induced seizure is known to produce lesions, activate microglia and induce inflammation in CA3. Upregulation of CGRP in these regions may well influence the immune and inflammatory responses to KA administration.

In the periphery, CGRP has been reported to control the production of cytokines by and the recruitment and migration of many types of immune cells, including dendritic cells (DC), and DC have been reported to express receptors for CGRP. The present study assessed the relationship between the response of bDC and the induction of CGRP expression in the hippocampal formation to a single injection of KA (30 mg/kg, i.p., 48 hrs), as compared to saline-injected controls. Tissue sections were stained with rabbit anti-CGRP antibody (1:8000; Sigma, St. Louis, Mo.) and an Alexa 647 conjugated secondary antibody. The number of bDC and the fluorescent intensity of CGRP-immunoreactivity (IR) were quantified in CA3 as well as the hilus and subgranular zone of the dentate gyrus (DG) (data not shown) using confocal microscopy. Images were acquired on a Zeiss 510 microscope and subsequently analyzed on Image J software (National Institutes of Health).

Following KA-induced seizures, EYFP+ bDC with activated morphology were clustered in regions of damage in dentate gyrus and in CA3 in close apposition to CGRP-expressing MFT. bDC also appeared to migrate into the hilus and subgranular zone of the dentate gyrus, a region that also expressed CGRP-IR (data not shown). These data suggest a role for CGRP in the recruitment and/or immune function of bDC in the hippocampal formation following KA-induced seizure.

### Example 7

### Activation of bDC Following Stroke

The experiments described in this Example were conducted to investigate if bDC are involved in the immune system response to damage caused by stroke.

### Materials and Methods

### Animals

CD11c-EYFP mice were obtained, bred, and maintained as described above. Subjects for the experiments in this Example included 8 male Itgax (CD11c)-EYFP Tg mice aged 2 to 3 months.

### Transient Medial Cerebral Artery Occlusion

Medial cerebral artery occlusion (MCAO) procedures were conducted as previously described (Cho et al., 2005; Kawano et al., 2006; Kunz et al., 2007). Mice were anesthetized with a mixture of isoflurane (1.5-2%), oxygen, and nitrogen. A fiber optic probe was glued to the parietal bone 2 mm posterior and 5 mm lateral to bregma, and connected to a laser-Doppler flowmeter (Periflux System 5000; Perimed, Jarfalla, Sweden) for continuous monitoring of cerebral blood flow (CBF). For MCAO (n=6), a heatblunted monofilament surgical suture (6-0) was inserted into the exposed external carotid artery, advanced into the internal carotid artery, and wedged into the circle of Willis to obstruct the origin of the MCA. The filament was left in place for 30 min and then withdrawn. Only animals that exhibited a reduction in CBF >85% during MCAO and a CBF recovery by >80% after 10 min of reperfusion were included in the study (Cho et al., 2005; Kunz et al., 2007). This procedure leads to reproducible infarcts similar in size and distribution to those reported by others using transient MCAO of comparable duration (Plesnila et al., 2001; Borsello et al., 2003). Sham animals (n=2) received identical anesthesia and surgical manipulations with no occlusion of the artery. Rectal temperature was monitored and kept constant (37.0+/−0.5° C.) during the surgical procedure and in the recovery period until the animals regained full consciousness.

### Perfusion, Sectioning, and Tissue Storage

Either 6 h or 3 days following reperfusion from MCAO or sham surgery, mice were deeply anesthetized with sodium pentobarbital (150 mg/kg, i.p.) followed by sequential intracardial perfusion with saline containing heparin (1 U/ml) and 4% paraformaldehyde in 0.1 M PB. The brains were removed and immersed in the same fixative for 6 hours at 4° C. For collection of free-floating tissue sections (40 μm) on the vibratome (n=1 for sham, n=2 for 3 day MCAO), brains were rinsed in PBS, sectioned and stored at −80° C. in a cryoprotectant solution containing 30% sucrose. For sectioning of brains on the cryostat (n=1 for sham, n=2 for 6 hr MCAO, and n=2 for 3 day MCAO) to better preserve the region of damage, fixed brains were immersed in 30% sucrose and frozen, sectioned at 20 μm or less, collected directly onto Superfrost Plus microscope slides (Fisher Scientific, Pittsburgh, Pa.), and stored at −80° C.

### Immunohistochemistry

Tissue sections, either free-floating or on slides, were rinsed with PB followed by TBS. Sections were blocked in 5% bovine serum albumin (BSA; Sigma, St. Louis, Mo.) for 1 hr RT, and all subsequent washes and incubations were conducted in TBS containing 0.1% BSA. All primary incubations occurred overnight at 4° C. in TBS-BSA containing 0.25% Triton-X. Primary antibodies included anti-rat Iba-1 (1:2000; Wako Chemicals, Richmond, Va.), anti-mouse CD45 (1:100; BD Biosciences, San Jose, Calif.), anti-mouse CD11c (clone N418, 1:200; eBiosciences, San Diego, Calif.), and anti-mouse MHC class II I-A/I-E (1:500; eBiosciences). Following extensive washing, sections were then incubated with the appropriate secondary antibody conjugated to Alexa 647 or Alexa 633 (1:1,000; Molecular Probes, Eugene, Oreg.) for 1 h RT. They were then washed, mounted (if necessary) and cover slipped for confocal microscopy analyses (Zeiss LSM 510).

### Results and Discussion

Following stroke, regions of brain damage can be roughly divided into a central core region and a surrounding penumbra region. In the core region, blood flow is severely compromised during stroke, leading to both early and extensive necrotic damage. The penumbra region comprises tissue surrounding the core area. In the penumbra region, cells are hypoperfused but still viable. Ongoing excitotoxicity, postischemic inflammation, and/or apoptosis may occur in the penumbra region over hours to days after stroke, potentially leading to infarction. Nevertheless, the viability of cells in the penumbra region immediately after stroke suggests a possibility for therapeutic intervention, for example, by suppressing inflammation and cell death in the penumbra.

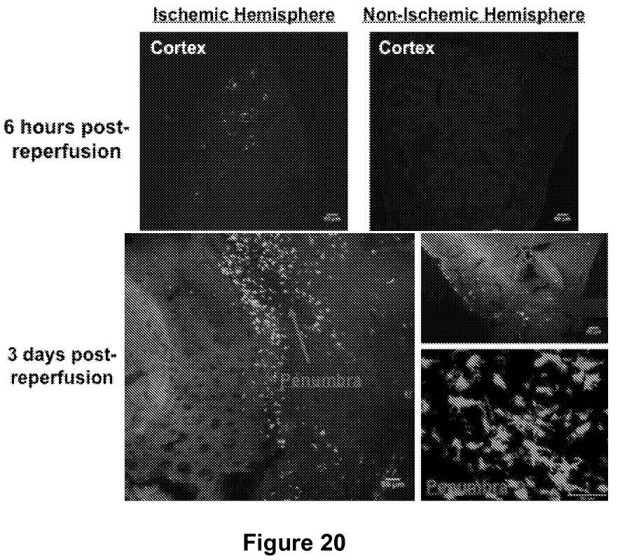

Stroke was induced in CD11c-EYFP transgenic mice using medial cerebral artery occlusion (MCAO). As early as six hours following MCAO-induced stroke, EYFP+ bDC were found to accumulate in both the core and penumbra regions (FIG. 20). By day 3 after MCAO, EYFP+ bDC had a pronounced presence in the penumbra. Penumbral bDC appeared to be mostly resident bDC, whereas bDC in the core had a very round morphology and appeared to have infiltrated from the periphery. The peripheral origin of bDC in the core were consistent with results reported by Ito et al. (2001) and Reichmann et al. (2002).

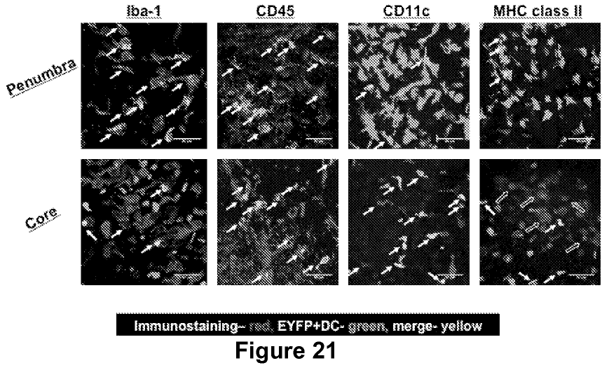

Marker phenotypes of activated bDC in both the penumbra and core were characterized by immunostaining. Penumbral EYFP+ bDC=were intermediate for CD45 and high for Iba-1. Expression of CD11c and MHC class II in penumbral EYFP+ bDC, were below the detection threshold for the antibodies used, though immunostaining of a small percentage of EYFP+ bDC resulted in detectable signals for CD11c and MHC class II.\ On the other hand, core bDC were Iba-1 low and high for CD45, CD11c, and MHC class II by immunostaining (FIG. 21).

It is noted that the level of CD11c protein expression necessary for positive immunostaining by the anti-CD11c antibody used in the present experiments is likely very high. RNA expression of CD11c may still be detectable even though CD11c protein may not be detectable. Thus, the CD11c promoter in penumbral bDC is active (as evidenced by CD11c promoter-driven expression of the EYFP transgene), but enough CD11c protein may not always accumulate to be detectable by immunostaining using available antibodies. mRNA expression of CD11c has been confirmed by microarray gene chip experiments (see Example 8) as well as by RT-PCR.

These results suggest that bDC may play a role in damage and/or repair after stroke and, given their presence in the penumbra region, may serve as targets for therapeutic intervention after a stroke.

### Example 8

### Gene Expression Analyses of bDC

In this example, gene expression profiles of bDC were obtained and analyzed in order to identify potential new markers of bDC. It is anticipated that results from these experiments may facilitate a better understanding of bDC function as well. CD11c-EYFP transgenic mice were obtained, maintained, and bred as described above. Materials and methods for gene expression analyses were similar to those described above in Example 1 (see “Real-time RT-PCR and gene array” and “reverse transcription and first-strand cDNA synthesis”).

### Results and Discussion

Gene expression profile experiments were performed on bDC from steady state brains from CD11c-EYFP transgenic mice. That is, bDC were obtained from brains that were not suffering from damage or in an inflammatory condition. EYFP+ brain cells were compared against EYFP− brain cells in one set of experiments. In another set of experiments, EYFP+ cells from brain were compared against EYFP+ cells from spleen. Presented in Table 2 are preliminary results listing some genes identified that were differentially expressed in EYFP+ brain cells. Genes may appear more than once in the list because they may be represented by more than one probe on the array.

TABLE 2
Differentially expressed genes in bDC

| | | EYFP+ vs. EYFP− | | EYFP+ brain |
|---|---|---|---|---|
| | | brain cells | | vs. EYFP+ |
| | | Fold | Fold | spleen cells |
| | Other | difference: | difference: | Fold |
| Gene | name | Gene chip 1 | Gene chip 2 | difference |
| Retnla | resistin-like | 108.8 | 175.8 | 164.9 |
| Retnla | resistin-like | 101.6 | 144.8 | 130.6 |
| CCL17 | | 36.0 | 31.7 | |
| CxCL9 | | 35.6 | 51.1 | |
| CD209a | Dc-Sign | 24.2 | 23.5 | |
| H2-Eb1 | MHCII | 21.6 | 14.5 | |
| Spp1 | Osteopontin | 21.2 | 17.8 | 3.1 |
| Axl | | 20.4 | 18.1 | 13.0 |
| H2-Aa | MHCII | 17.6 | 17.0 | |
| H2-Ab1 | MHCII | 16.8 | 15.5 | |
| CxCL2 | MIP-2 | 14.9 | 11.3 | 12.3 |
| ClecVa | Dectin-1 | 12.0 | 9.3 | |
| CCR2 | | 10.9 | 9.7 | 12.4 |
| Itgax | CD11c | 7.7 | 7.0 | |
| IGF-1 | | 7.3 | 7.3 | 7.6 |
| CD36 | | 5.6 | (not | |
| | | | repeated) | |

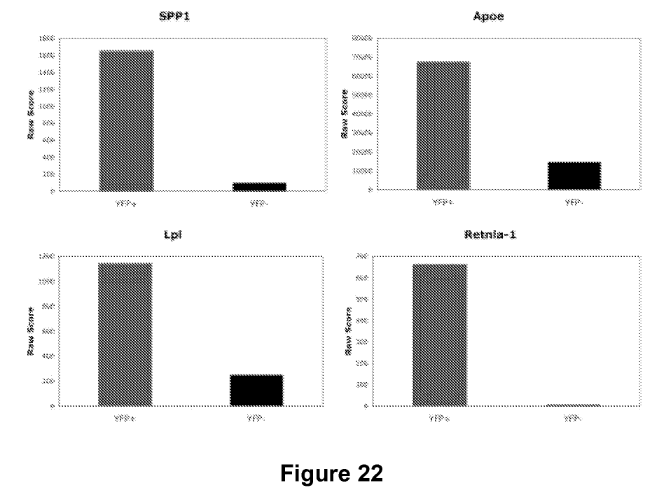

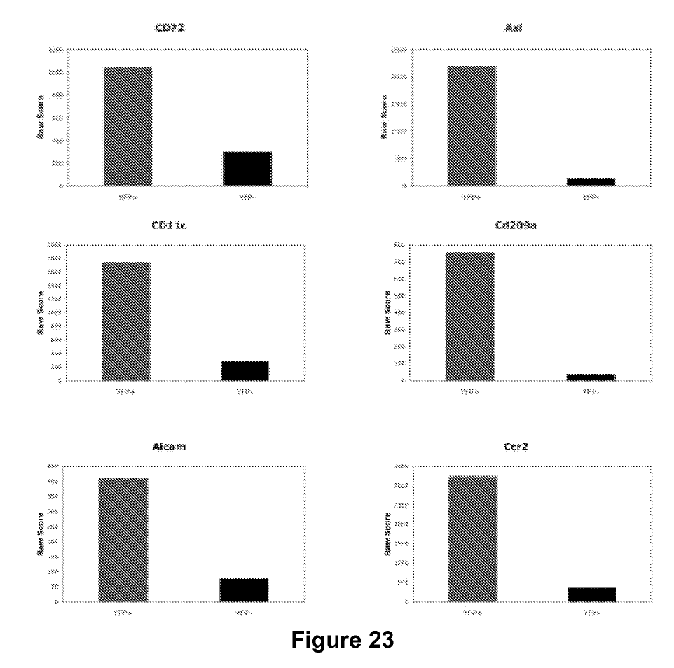

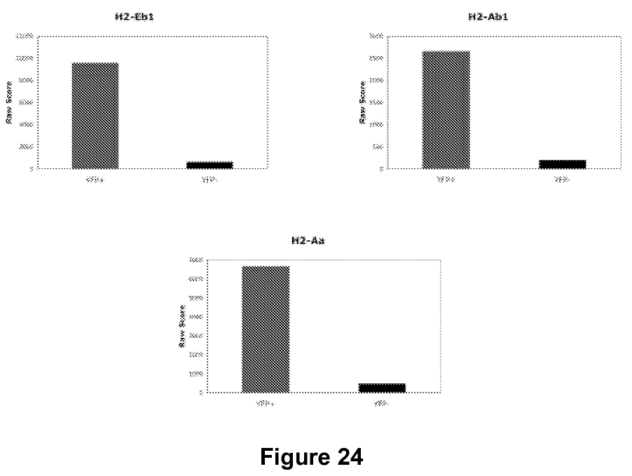

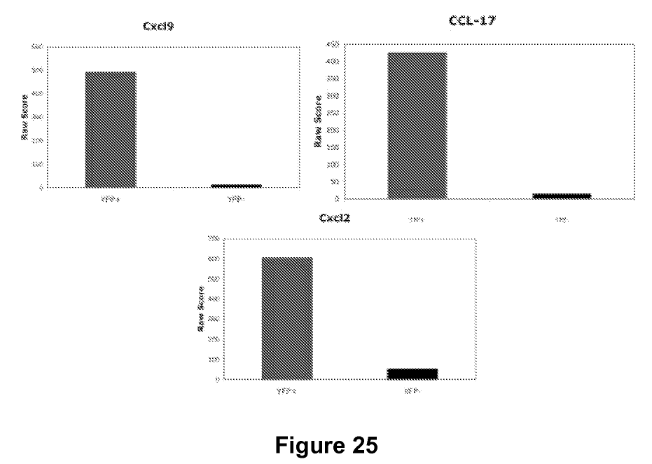

As shown in Table 2 and FIGS. 22-25, differentially regulated genes included genes involved in several different classifications, including secretion, cell surface receptors, antigen presentation, and chemokines. As expected, Itgax was overexpressed in bDC. DC-SIGN, which was known to be expressed in a subset of dendritic cells, was expressed in bDC at levels 20-fold higher than the expression levels observed in microglia. A particularly notable gene identified in the microarray experiments was the resistin-like alpha gene, which was overexpressed over a hundred fold in bDC. Resistin-like alpha is known to be highly expressed in white adipocytes and is thought to have a role in insulin resistance and inflammation.

### Example 9

### Functional Characterization of bDC

Experimental results discussed above suggest that bDC could participate in traumatic events in the brain such as seizure and stroke (see Examples 5-7 above). bDC could play beneficial, harmful, or both beneficial and harmful roles in such events. It is postulated, without wishing to be bound by any particular theory, that bDC may play certain roles depending on their context. The experiments in this Example are directed toward examining the known attributes of peripheral DC in bDC to determine if bDC function as peripheral DC do. In experiments described below and in ongoing experiments, bDC have been or are being analyzed for expression of gene products involved in antigen presentation, migration from the CNS to the periphery, ability to activate naïve T-cells, and transportation of antigen from the brain to the periphery.

### Materials and Methods

CD11c-EYFP mice were obtained, maintained, and bred as described above.

Isolation of Adult Brain Dendritic Cell (bDC) by Fluorescence Activated Cell Sorting (FACS)

Slight variations on previously reported methods to obtain a single population of brain leukocytes by FACS were used. Adult mice (2-3 mo.) were rapidly decapitated and brains were removed and placed on ice in Hank's balanced salt solution (Gibco, Carlsbad, Calif.). Meninges, blood vessels and choroid plexus were carefully removed under a dissecting scope and brain cell suspensions were incubated with type II-S collagenase (600 U; Sigma) and DNAse (450 U; Invitrogen, Carlsbad, Calif.) for 30 min at 37° C. in 15 mL HBSS (w/CaMg²⁺). After digestion by collagenase, brains were homogenized by repetitive gentle pipetting with fire-polished Pasteur pipettes on ice followed by filtering through a 40 μm cell strainer (BD, Franklin Lakes, N.J.). Cells were washed by centrifugation and subjected to a 70%-37% Percoll gradient centrifugation. Cells collected from the 37/70 interphase were washed and re-suspended in 5% FBS-PBS containing 10 ng/ml DAPI before sorting in a FACS Aria Flow Cytometer (BD).

### Primary Microglia/bDC Cultures

Cell cultures were prepared following standard protocols. 2-day-old mouse pup brains were dissected on ice, and meninges were carefully removed. Forebrains were minced in 5% FBS-PBS, dissociated using fire polished Pasteur pipettes, and then passed through a 40 μm nylon cell strainer (BD). Cells were washed once in buffer and seeded in culture media at a density of roughly two forebrains per 75 mm flask. Cells were cultured in 10% FBS DMEM (Gibco) supplemented with 5 ng/ml GM-CSF (Sigma), at 37° C., 5% CO₂. Media was changed every 4-5 days. After 10-14 days in culture, cells were shaken at 125 rpm for 3-5 hours at 37° C. to harvest detached cells.

For selection of EYFP+ bDCs, cells were sorted using a FACS Aria Cytometer (BD). Later, EYFP+ (and/or negative microglia) were seeded in 6-well plates at a density of 1 million cells per well (for Western Blots), in 24-well assay plates at a density of 0.25-0.3 million cells per well (for cytokine assays), or in 24-well plates at a density of 0.02 million cells per well (for immunocytochemistry). The following day, cells were rinsed and incubated for 24 hours at 37° C. with vehicle or stimulated with 100 ng/ml LPS+10 ng/ml INFγ, or 10 ng/ml INFγ alone in 1% FBS DMEM+GMCSF.

### Immunocytochemistry

Cells were seeded onto Poly-L coated glass cover-slips in a 24-well plate (2×10⁴ cells/well) and cultured overnight. Cells were fixed in 4% paraformaldehyde PBS, permeabilized, and blocked in 5% goat serum PBS, 0.1% Tween (Sigma) for 1 hour at room temperature. Primary antibodies incubations were done overnight at 4° C. in blocking buffer with rat anti MHC-II antiserum (1:500) (eBioscience). Cells were washed 5 times in 0.1% Tween PBS, and then incubated for 1 hour at room temperature with DAPI (1:5000) and goat anti-rat fluorescent secondary antibodies coupled to Alexa-633 (1:1000) (Molecular Probes). Coverslips were washed 5 times in 0.1% Tween PBS and then mounted on glass slides using Dako fluorescent mounting media (Dako, Carpinteria, Calif.) for microscopy. Confocal images were acquired using a LSM510 confocal Zeiss Axioplan microscope with a kripton/argon laser and a HeNe laser (Rockfeller University Bioimaging Facility).

### Western Blotting

Cultured microglia were rinsed in cold PBS supplemented with Ca²⁺ and Mg²⁺ and then resuspended in protein lysis buffer (6M Urea, 20 mM Tris-HCl pH7.5, 2% SDS, 10% glycerol, 1% protease inhibitor cocktail; Sigma) supplemented with phosphatase inhibitor (1 mM NaVO₄; Sigma). Cells were sonicated to homogeneity and then quantified using the BioRad Dc protein assay (BioRad; Hercules, Calif.). Samples were stored at −20° C. until processed by Western blot. Equal amounts of protein were mixed with Laemmli loading buffer, heated at 70° C. for 10 min, and separated by SDS-PAGE performed under reducing conditions with 4-12% acrylamide NuPage gels according to manufacturer's instructions (Invitrogen). Resolved proteins were transferred to PVDF membranes (Invitrogen). Membranes were rinsed in 0.1 M Tris-Buffered Saline with 0.1% Tween-20 (TBS-T) and blocked with a solution of 5% non-fat dry milk in TBS-T for 1 hour at room temperature on an orbital shaking platform. Membranes were washed with TBS-T and incubated overnight at 4° C. with rat anti-MHC-II (1:1,000) diluted in 5% BSA in TBS-T solution. Membranes were washed and incubated with biotin-conjugated species-specific anti-antiserum (1:1,000) in blocking solution. Membranes were again washed and incubated with Horseradish Peroxidase-conjugated anti-biotin:antibody (1:1,000) in blocking solution. Washed membranes were developed with SuperSignal West Pico substrate (Pierce, Rockford, Ill.) and then exposed to X-Ray film (X-OMAT AR; Kodak, Rochester, N.Y.). To control for protein loading, membranes were incubated in Restore Western Blot Stripping Buffer (Pierce), washed, and immunoblotted as described above using an anti-actin antibody (Sigma; 1:40,000). Developed films were analyzed for densitometry using a computerized image analysis software (MCID-M4; Imaging Research, Inc., St. Catherines, ON), and the data was normalized as follows: 11bHSD-1 protein band (density*area)/actin (density*area).

Cytokine Production in bDCs and MG

Cells were stimulated for 24 hours with 100 ng/ml LPS+10 ng/ml INFγ in 1% FBS DMEM. Supernatants were collected and frozen at −20° C. TNFα and IL-6 were quantified by ELISA following manufacturer's instructions (eBioscience, CA), and NO was quantified using the Greiss assay (Promega, Madison, Wis.).

### Stereotaxic Intracerebral Injections

Animals were anesthetized by 3% isofluorane ventilation and placed in a mouse stereotaxic frame. After surgical exposure and careful drilling of the skull, a 10 μL FlexiFil microsyringe (500819 World Precision Instruments) was lowered to the desired coordinate. INFγ infusion (0.5 μL) was administered in the CA2 area (Bregma coordinates: Rostro-Caudal −2.3; Medial-Lateral −2.7; Dorso-Ventral −2.2) over a period of 2 minutes after which the needle was left in place for another 5 minutes before removal. For cell injections, 0.5 μL (Corpus Collosum, Bregma Rostro-Caudal −2.3; Medial-Lateral −0.6; Dorso-Ventral −1.2) or 0.5 μL (Lateral Ventricle, Bregma Rostro-Caudal −0.5; Medial-Lateral −1.0; Dorso-Ventral −2.2) of an EYFP+ bDC cell suspension (10 million cells/mL in PBS) was infused as described above. Animal recovery was monitored 30 minutes post-surgery and then returned to single-housing with water and food ad libitum. Surgery was performed under sterile conditions. Mice were sacrificed 3 or 7 days post-injections.

### Perfusion

For light and fluorescent microscopy, mice were deeply anesthetized with sodium pentobarbital (150 mg/kg, i.p.) followed by sequential perfusion through the ascending aorta with saline, containing heparin (1 unit/mL), and with 4% paraformaldehyde (PFA) in 0.1 M phosphate buffer (PB). The brains were removed and immersed in the same fixative overnight at 4° C. Brains were rinsed in PBS and allowed to saturate overnight in cryoprotectant (25% ethylene glycol, 25% glycerol in 0.1 M PB, pH 7.4). 30 μm coronal sections were cut on a Leica vibrotome (Microm, Walldorf, Germany), serially collected and stored at −20° C. in cryoprotectant until use.

### Brain Immunohistochemistry

Free-floating fixed coronal sections were processed for the immunocytochemical localization of EYFP according to the avidin-biotin complex (ABC) procedure. Sections were incubated in: (1) 0.5% BSA in 0.1 M Tris-Buffered Saline (pH 7.6; TBS) for 30 min; (2) in chicken anti-EYFP antiserum (1:11 000) in 0.1% BSA in TBS for 1 day at room temperature followed by an additional 2 days at 4° C.; (3) biotinylated goat anti-chicken IgG in 0.1% BSA (1:400, Vector Laboratories) for 1 hour; (4) peroxidase-avidin complex for 30 min and (5) diaminobenzidine (DAB peroxidase substrate kit, Vector, Burlingame, Calif.). Sections were mounted on 1% gelatin coated slides, air dried, dehydrated, and cover-slipped with DPX mounting medium (Aldrich) and photographed using a Nikon Optiphot Microscope with a Coolpix Digital camera.

### Generation of Bone Marrow (BM) and Fetal Liver (FL) Chimeras

### Irradiation

Mice were fed antibiotic food or water (e.g. Sulfatrim) for two weeks prior to the irradiation. Lethal irradiation of recipient mice was performed using a cobalt irradiator within the barrier facility. Mice were placed in the irradiator to deliver a dose of 450R for 4 minutes. The same dose of irradiation was repeated 3 hours later for a total dose of 900R (split dose irradiation is used to limit the non-hematopoietic toxicity). Mice were monitored on the day after irradiation and 3-4 times a week for the first 2 weeks after the second treatment for signs of acute illness.

### Bone Marrow/Fetal Liver Preparation

Bone marrow or fetal liver cells were isolated from euthanized donor mice. Bone marrow or fetal liver cells of a desired mouse strain were transferred into lethally irradiated recipient mice as described below.

Bone marrow cells were aseptically harvested by flushing femurs with Dulbecco's phosphate-buffered saline (DPBS). The samples were combined, filtered through a 40 μm nylon mesh and centrifuged. Recovered cells were resuspended in DPBS at a concentration of 17×10⁶ viable nucleated cells per 200 microliters.

Fetal liver cells were prepared from timed pregnant embryonic day 16 mice (E16). Embryos were removed from the uterus and screened for transgene expression using a fluorescent light. A single-cell suspension was prepared from the fetal livers of the EYFP-positive embryos by mechanical dissociation and passing the suspension through a 40 μm cell strainer. Cells were washed with RPMI containing 5% FCS, and cell density was adjusted to 25×10³ cells/μL in DPBS.

### Injection of Bone Marrow/Fetal Liver Cells

Irradiated mice were injected within 24 hours after the second irradiation via the tail vein with donor bone marrow (1−5×10⁶) or fetal liver (0.5−5×10⁶) cells in 200-400 μl of sterile phosphate buffered saline. Recipient mice were maintained on antibiotic-containing water or feed for 4 weeks. Duration of the entire procedure is typically 6-12 weeks.

### Results and Discussion

Brain cells from CD11c-EYFP+ PN2 mice were cultured in GMCSF for 10 days and then sorted by FACS into EYFP+ (bDC) and EYFP− (microglia) populations. Sorted cell populations were cultured in medium and stimulated with LPS and INFγ, and then analyzed in various experiments as follows.

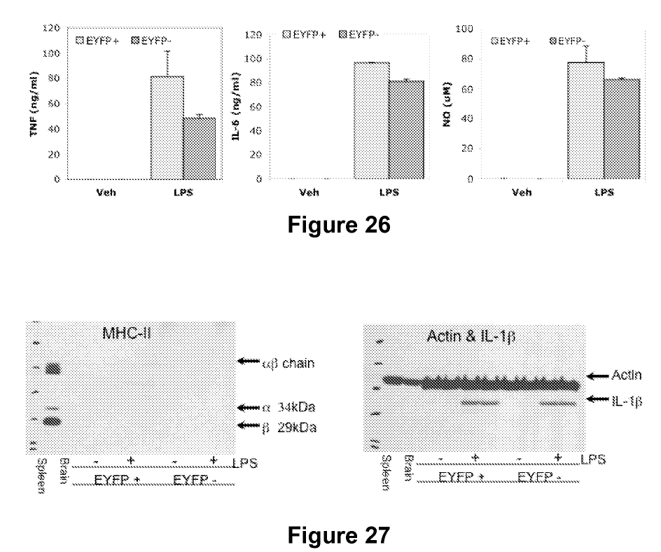

Cytokine production from LPS and INFγ-stimulated cells was measured by ELISA. As depicted in FIG. 26, bDC produced TNF, IL-6, and NO at levels comparable to or higher than did microglia.

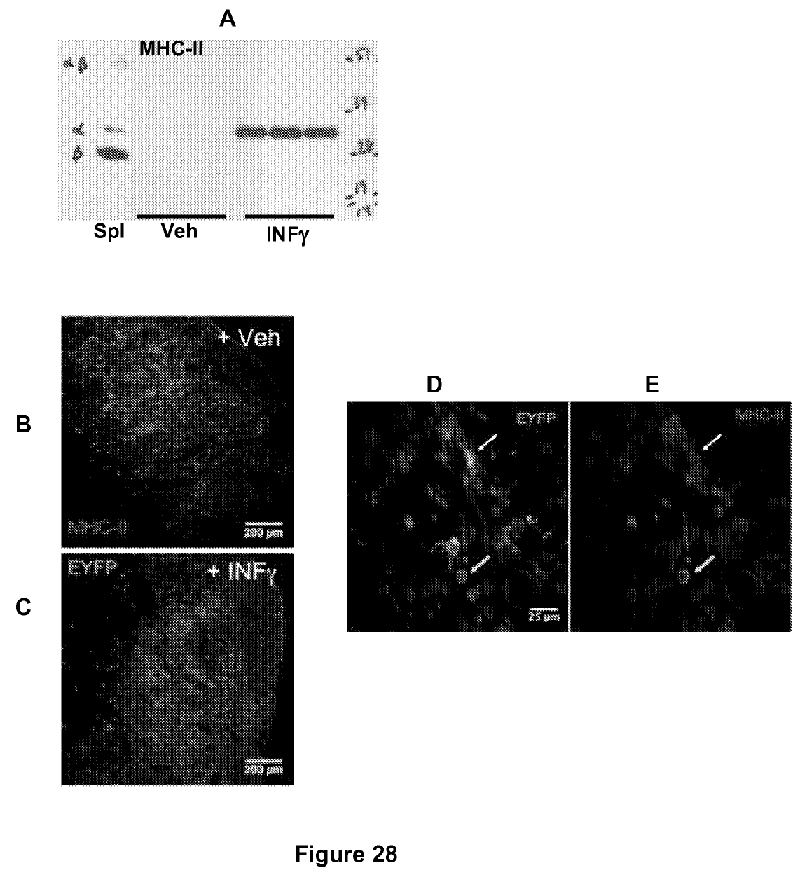

Unstimulated EYFP+ bDC did not express MHC class II molecules or IL-1β that could be detectable by Western blot. When stimulated by LPS, both EYFP+ bDC and EYFP− microglia expressed IL-1β, but neither expressed MHC class II (FIG. 27). INFγ, on the other hand induced MHC II expression in cultured bDC (FIG. 28A).

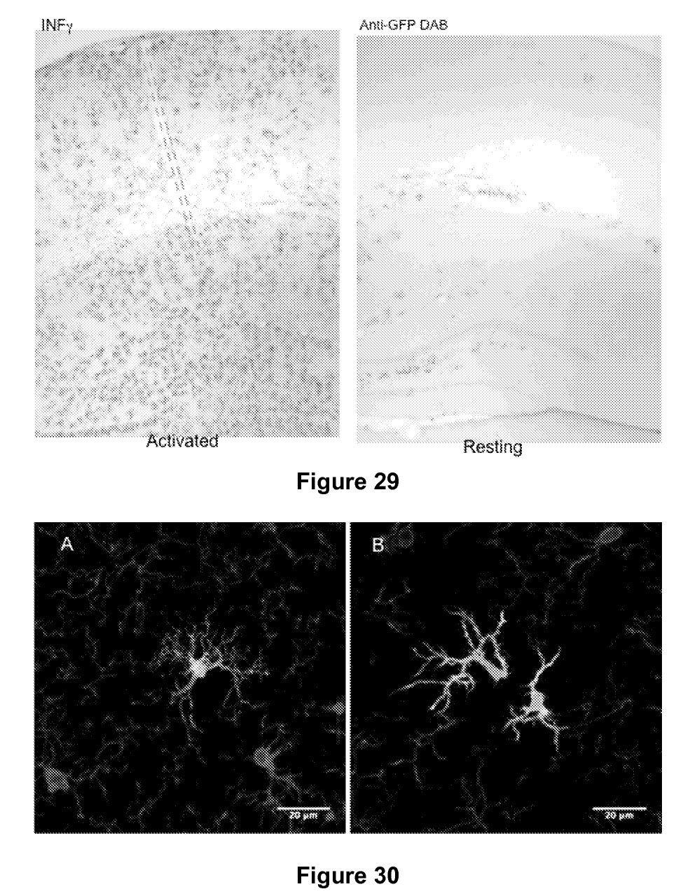

To determine if MHC class II expression can be induced in bDC in vivo, INFγ was injected intracerebrally into adult mice. INFγ induced MHC class II expression that colocalized with EYFP staining (FIG. 28B-E). No MHC class II staining was observed in resting brain. In cervical lymph nodes, INFγ-stimulated MHC class II expression also colocalized with YFP (data not shown). Furthermore, intracerebral injection of INFγ into the brain led to a massive increase in numbers of bDC (see FIG. 29). The increase in bDC following INFγ stimulation was observed in as few as three days after injection.

These results demonstrate that bDC have antigen presenting machinery. They can be induced to express cytokines and can be stimulated to express MHC class II. bDC are rapidly and greatly activated in the brain following stimulation by IFNγ.

To determine if bDC that were present after IFNγ induction were resident in the brain or recruited from the periphery, bone marrow chimeras were used to distinguish resident and peripheral bDC. In such experiments, bone marrow or fetal liver cells from EYFP+ transgenic mice were transferred into irradiated EYFP− recipients or vice versa. In such chimeras, peripheral DC originate from transferred cells and are distinguishable from resident bDC, which originate from the host. Interestingly, DC that are recruited from the periphery are morphologically distinguishable from DC that reside in the brain (FIG. 30).

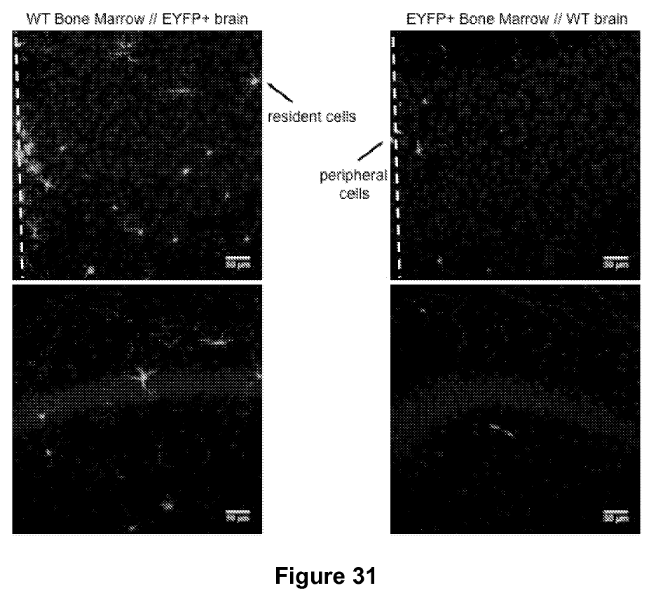

In a EYFP+ host/wild type bone marrow chimera, injection with INFγ led to activation of EYFP+ cells. In a wild type host/EYFP− bone marrow chimera, INFγ-activated cells were EYFP− (FIG. 31). These results indicate that INFγ-activated cells are resident bDC.

Ongoing experiments are directed to investigating the motility of bDC (in particular, whether they are able to migrate from the CNS into the periphery) and whether bDC can activate T-cells.

### Other Embodiments

Other embodiments of the invention will be apparent to those skilled in the art from a consideration of the specification or practice of the invention disclosed herein. It is intended that the specification and examples be considered as exemplary only, with the true scope of the invention being indicated by the following claims.

### REFERENCES

Agger R, Crowley M T, Witmer-Pack M D. 1990. The surface of dendritic cells in the mouse as studied with monoclonal antibodies. Int Rev Immunol 6: 89-101.
Altman J. 1969. Autoradiographic and histological studies of postnatal neurogenesis. IV. Cell proliferation and migration in the anterior forebrain, with special reference to persisting neurogenesis in the olfactory bulb. J Comp Neurol 137: 433-157.
Atlas Navigator, YTIG, Inc. 2000. Atlas from Comparative Cytoarchitectonic Atlas of the 129/Sv and C57BL/6 Mouse Brains. New York: Elsevier.
Austyn J M, Gordon S. 1981. F4/80, a monoclonal antibody directed specifically against the mouse macrophage. Eur J Immunol 11: 805-815.
Bailey S L, Carpentier P A, McMahon E J, Begolka W S, Miller S D. 2006. Innate and adaptive immune responses of the central nervous system. Crit. Rev Immunol 26: 149-188.
Becher B, Antel J P. 1996. Comparison of phenotypic and functional properties of immediately ex vivo and cultured human adult microglia. Glia 18: 1-10.
Bonifaz L, Bonnyay D, Mahnke K, Rivera M, Nussenzweig M C, Steinman R M. 2002. Efficient targeting of protein antigen to the dendritic cell receptor DEC-205 in the steady state leads to antigen presentation on major histocompatibility complex class I products and peripheral CD8+ T cell tolerance. J Exp Med 196: 1627-1638.
Borsello T, Clarke P G, Hirt L, Vercelli A, Repici M, Schorderet D F, Bogousslavsky J, Bonny C (2003) A peptide inhibitor of c-Jun N-terminal kinase protects against excitotoxicity and cerebral ischemia. Nat Med 9:1180-1186.
Brocker t. Riedinger M. Karjalainen K. 1997 Driving gene expression specifically in dendritic cells. Adv Exp Med Biol 417: 55-57.
Brown J P, Couillard-Després S, Cooper-Kuhn C M, Winkler J, Aigner L, Kuhn H G. 2003. Transient expression of doublecortin during adult neurogenesis. J Comp Neurol 467: 1-10.
Butovsky O, Talpalar A E, Ben-Yaakov K, Schwartz M. 2005. Activation of microglia by aggregated beta-amyloid or lipopolysaccharide impairs MHC-II expression and renders them cytotoxic whereas IFN-gamma and IL-4 render them protective. Mol Cell Neurosci 29: 381-393.
Butovsky O, Koronyo-Hamaoui M, Kunis G, Ophir E, Landa G, Cohen H, Schwartz M. 2006a. Glatiramer acetate fights against Alzheimer's disease by inducing dendritic-like microglia expressing insulin-like growth factor 1. Proc Natl Acad Sci USA 103: 11784-11789.
Butovsky O, Ziv Y, Schwartz A, Landa G, Talpalar A E, Pluchino S, Martino G, Schwartz M. 2006b. Microglia activated by IL-4 or IFN-gamma differentially induce neurogenesis and oligodendrogenesis from adult stem/progenitor cells. Mol Cell Neurosci 31: 149-160.
Butovsky O, Kunis G, Koronyo-Hamaoui M, Schwartz M. 2007. Selective ablation of bone marrow-derived dendritic cells increases amyloid plaques in a mouse Alzheimer's disease model. Eur J Neurosci 26: 413-416.
Carson M J, Reilly C R, Sutcliffe J G, Lo D. 1998. Mature microglia resemble immature antigen-presenting cells. Glia 22: 72-85.
Chan J, Aoki C, Pickel V M. 1990. Optimization of differential immunogold-silver and peroxidase labeling with maintenance of ultrastructure in brain sections before plastic embedding. J Neurosci Methods 33: 113-127.
Chang S C, Kao M, Fu M, Lin C. 2001. Modulation of NO and cytokines in microglia cells by CU/ZN-superoxide dismutase. Free Radic Biol Med 31: 1084-1089.
Cho S, Park E M, Febbraio M, Anrather J, Park L, Racchumi G, Silverstein R L, Tadecola C (2005) The class B scavenger receptor CD36 mediates free radical production and tissue injury in cerebral ischemia. J Neurosci 25:2504-2512.
Cohen I R, Schwartz M. 1999. Autoimmune maintenance and neuroprotection of the central nervous system. J Neuroimmunol 100: 111-114.
Cohen P E, Hardy M P, Pollard J W. 1997. Colony-stimulating factor-1 plays a major role in the development of reproductive function in male mice. Mol Endocrinol 11: 1636-1650.
Curtin J F, King G D, Barcia C, Liu C, Hubert F X, Guillonneau C, Josien R, Anegon I, Lowenstein P R, Castro M G. 2006. Fms-like tyrosine kinase 3 ligand recruits plasmacytoid dendritic cells to the brain. J Immunol 176: 3566-3577.
El-Nefiawy N, Abdel-Hakim K, Kanayama N, Terao T. 2005. Dendritic cell differentiation and maturation in embryonic rat liver: immunohistochemistry and electron microscopy with reference to dendritic cell contacts. Anat Histol Embryol 34: 72-79.
Encinas J M, Vaahtokari A, Enikolopov G. 2006. Fluoxetine targets early progenitor cells in the adult brain. Proc Natl Acad Sci USA 103: 8233-8238.
Farrar C E, Huang C S, Clarke S G, Houser C R. 2005. Increased cell proliferation and granule cell number in the dentate gyrus of protein repair-deficient mice. J Comp Neurol 493: 524-537.
Fischer H G, Bielinsky A K. 1999. Antigen presentation function of brain-derived dendriform cells depends on astrocyte help. Int Immunol 11: 1265-2574.
Fischer H G, Reichmann G. 2001. Brain dendritic cells and macrophages/microglia in central nervous system inflammation. J Immunol 166: 2717-2726.
Fischer H G, Bonifas U, Reichmann G. 2000. Phenotype and functions of brain dendritic cells emerging during chronic infection of mice with Toxoplasma gondii. J Immunol 164: 4826-4834.
Franklin K B, Paxinos G. 1997. The mouse brain in stereotaxic coordinates. San Diego: Academic Press.
Ganong W F. 2000. Circumventricular organs: definition and role in the regulation of endocrine and autonomic function. Clin Exp Pharmacol Physiol 27: 422-427.
Gleeson J G, Allen K M, Fox J W, Lamperti E D, Berkovic S, Scheffer I, Cooper E C, Dobyns W B, Minnerath S R, Ross M E, Walsh C A. 1998. Doublecortin, a brain-specific gene mutated in human X-linked lissencephaly and double cortex syndrome, encodes a putative signaling protein. Cell 92: 63-72.
Gould E, Cameron H A. 1996. Regulation of neuronal birth, migration and death in the rat dentate gyrus. Dev Neurosci 18: 22-35.
Gregerson D S, Sam T N, McPherson S W. 2004. The antigen-presenting activity of fresh, adult parenchymal microglia and perivascular cells from retina. J Immunol 172: 6587-6597.
Greter M, Heppner F L, Lemos M P, Odermatt B M, Goebels N, Laufer T, Noelle R J, Becher B. 2005. Dendritic cells permit immune invasion of the CNS in an animal model of multiple sclerosis. Nat Med 11: 328-334.
Hassan N F, Rifat S, Campbell D E, McCawley L J, Douglas S D. 1991. Isolation and flow cytometric characterization of newborn mouse brain-derived microglia maintained in vitro. J Leukoc Biol 50: 86-92.
Hatterer E, Davoust N, Didier-Bazes M, Vuaillat C, Malcus C, Belin M F, Nataf S. 2006. How to drain without lymphatics? Dendritic cells migrate from the cerebrospinal fluid to the B-cell follicles of cervical lymph nodes. Blood 107: 806-812.
Hsu S M, Raine L, Fanger H. 1981. Use of avidin-biotin-peroxidase complex (ABC) in immunoperoxidase techniques: a comparison between ABC and unlabeled antibody (PAP) procedures. J Histochem Cytochem 29: 577-580.
Hume D A, Perry V H, Gordon S. 1983. Immunohistochemical localization of a macrophage-specific antigen in developing mouse retina: phagocytosis of dying neurons and differentiation of microglia cells to form a regular array in the plexiform layers. J Cell Biol 97: 253-257.
Hume D A, Perry V H, Gordon S. 1984. The mononuclear phagocyte system of the mouse defined by immunohistochemical localisation of antigen F4/80: macrophages associated with epithelia. Anat Rec 210: 503-512.
Iribarren P, Cui Y H, Le Y, Wang J M. 2002. The role of dendritic cells in neurodegenerative diseases. Arch Immunol Ther Exp 50: 187-96.
Ito D, Imai Y, Ohsawa K, Nakajima K, Fukuuchi Y, Kohsaka S. 1998. Microglia-specific localisation of a novel calcium binding protein, Iba1. Brain Res Mol Brain Res 57: 1-9.
Ito D, Tanaka K, Suzuki S, Dembo T, Fukuuchi Y. 2001. Enhanced Expression of Iba1, Ionized Calcium-Binding Adapter Molecule 1, After Transient Focal Cerebral Ischemia In Rat Brain. Stroke 32(5):1208-15.
Jessberger S, Romer B, Babu H, Kempermann G. 2005. Seizures induce proliferation and dispersion of doublecortin-positive hippocampal progenitor cells. Exp Neurol 196: 342-351.
Jouandet M L, Hartenstein V. 1983. Basal telencephalic origins of the anterior commissure of the rat. Exp Brain Res 50: 183-192.
Jung S, Unutmaz D, Wong P, Sano G, De los Santos K, Sparwasser T, Wu S, Vuthoori S, Ko K, Zavala F, Pamer E G, Littman D R, Lang R A. 2002. In vivo depletion of CD11c+ dendritic cells abrogates priming of CD8+ T cells by exogenous cell-associated antigens. Immunity 17: 211-220.
Karman J, Ling C, Sandor M, Fabry Z. 2004. Initiation of immune responses in brain is promoted by local dendritic cells. J Immunol 173: 2353-2361.
Karman J, Chu H H, Co D O, Seroogy C M, Sandor M, Fabry Z. 2006. Dendritic cells amplify T cell-mediated immune responses in the central nervous system. J Immunol 177: 7750-7760.
Kawano T, Anrather J, Zhou P, Park L, Wang G, Frys K A, Kunz A, Cho S, Orio M, Iadecola C (2006) Prostaglandin E(2) EP1 receptors: downstream effectors of COX-2 neurotoxicity. Nat Med 12:225-229.
Kipnis J, Schwartz M. 2005. Controlled autoimmunity in CNS maintenance and repair: naturally occurring CD4+ CD25+ regulatory T-cells at the crossroads of health and disease. Neuromol Med 7: 197-206.
Kunz A, Park L, Abe T, Gallo E F, Anrather J, Zhou P, Iadecola C (2007) Neurovascular protection by ischemic tolerance: role of nitric oxide and reactive oxygen species. J Neurosci 27:7083-7093.
Lein E S, Hawrylycz M J, Ao N, Ayres M, Bensinger A, Bernard A, Boe A F, Boguski M S, Brockway K S, Byrnes E J, Chen L, Chen L, Chen T M, Chin M C, Chong J, Crook B E, Czaplinska A, Dang C N, Datta S, Dee N R, Desaki A L, Desta T, Diep E, Dolbeare T A, Donelan M J, Dong H W, Dougherty J G, Duncan B J, Ebbert A J, Eichele G, Estin L K, Faber C, Facer B A, Fields R, Fischer S R, Fliss T P, Frensley C, Gates S N, Glattfelder K J, Halverson K R, Hart M R, Hohmann J G, Howell M P, Jeung D P, Johnson R A, Karr P T, Kawal R, Kidney J M, Knapik R H, Kuan C L, Lake J H, Laramee A R, Larsen K D, Lau C, Lemon T A, Liang A J, Liu Y, Luong L T, Michaels J, Morgan J J, Morgan R J, Mortrud M T, Mosqueda N F, Ng L L, Ng R, Orta G J, Overly C C, Pak T H, Parry S E, Pathak S D, Pearson O C, Puchalski R B, Riley Z L, Rockett H R, Rowland S A, Royall J J, Ruiz M J, Sarno N R, Schaffnit K, Shapovalova N V, Sivisay T, Slaughterbeck C R, Smith S C, Smith K A, Smith B I, Sodt A J, Stewart N N, Stumpf K R, Sunkin S M, Supram M, Tam A, Teemer C D, Thaller C, Thompson C L, Varnam L R, Visel A, Whitlock R M, Wohnoutka P E, Wolkey C K, Wong V Y, Wood M, Yaylaoglu M B, Young R C, Youngstrom B L, Yuan X F, Zhang B, Zwingman T A, Jones A R. 2007. Genome-wide atlas of gene expression in the adult mouse brain. Nature 445: 168-176.
Lin H H, Faunce D E, Stacey M, Terajewicz A, Nakamura T, Zhang-Hoover J, Kerley M, Mucenski M L, Gordon S, Stein-Streilein J. 2005. The macrophage F4/80 receptor is required for the induction of antigen-specific efferent regulatory T cells in peripheral tolerance. J Exp Med 201: 1615-1625.
Lind D, Franken S, Kappler J, Jankowski J, Schilling K. 2005. Characterization of the neuronal marker NeuN as a multiply phosphorylated antigen with discrete subcellular localization. J Neurosci Res 79: 295-302.
Lindquist R L, Shakhar G, Dudziak D, Wardemann H, Eisenreich T, Dustin M L, Nussenzweig M C. 2004. Visualizing dendritic cell networks in vivo. Nat Immunol 5: 1243-1250.
Liu X F, Fawcett J R, Thorne R G, DeFor T A, Frey W H 2nd. 2001. Intranasal administration of insulin-like growth factor-I bypasses the blood-brain barrier and protects against focal cerebral ischemic damage. J Neurol Sci 187: 91-97.
Livak K J, Schmittgen T D. 2001. Analysis of relative gene expression data using real-time quantitative PCR and the 22deltadelta CT method. Methods 25: 402-408.
Lothman E W, Collins R C. 1981. Kainic acid induced limbic seizures: metabolic, behavioral, electroencephalographic and neuropathological correlates. Brain Res 218: 299-318.
Lowhagen P, Johansson B B, Nordborg C. 1994. The nasal route of cerebrospinal fluid drainage in man. A light-microscope study. Neuropathol Appl Neurobiol 20: 543-550.
McClintock M K, Jacob S, Zelano B, Hayreh D J. 2001. Pheromones and vasanas: the functions of social chemosignals. Nebr Symp Motiv 47: 75-112.
McMahon E J, Bailey S L, Miller S D. 2006. CNS dendritic cells: critical participants in CNS inflammation? Neurochem Int 49: 195-203.
McMenamin P G. 1999. Distribution and phenotype of dendritic cells and resident tissue macrophages in the dura mater, leptomeninges, and choroid plexus of the rat brain as demonstrated in wholemount preparations. J Comp Neurol 405: 553-62.
Menalled and Chesselet, 2002. “Mouse models of Huntington's disease.” Trends Pharmacol Sci. 23(1):32-9
Mercier F, Kitasako J T, Hatton G I. 2002. Anatomy of the brain neurogenic zones revisited: fractones and the fibroblast/macrophage network. J Comp Neurol 451: 170-88.
Metlay J P, Witmer-Pack M D, Agger R, Crowley M T, Lawless D, Steinman R M. 1990. The distinct leukocyte integrins of mouse spleen dendritic cells as identified with new hamster monoclonal antibodies. J Exp Med 171: 1753-1771.
Mikami Y, Okano H, Sakaguchi M, Nakamura M, Shimazaki T, Okano H J, Kawakami Y, Toyama Y, Toda M. 2004. Implantation of dendritic cells in injured adult spinal cord results in activation of endogenous neural stem/progenitor cells leading to de novo neurogenesis and functional recovery. J Neurosci Res 76: 453-465.
Mullen R J, Buck C R, Smith A M. 1992. NeuN, a neuronal specific nuclear protein in vertebrates. Development 116: 201-211.
Muramoto T, Kitamoto T, Tateishi J, Goto I. 1992. The sequential development of abnormal prion protein accumulation in mice with Creutzfeldt-Jakob disease. Am J Pathol 140: 1411-1420.
Nacher J, Crespo C, McEwen B S. 2001. Double cortin expression in the adult rat telencephalon. Eur J Neurosci 14: 629-44.
Nagai T, Keiji I, Park E S. 2002. A variant of yellow fluorescent protein with fast and efficient maturation for cell-biological applications. Nat Biotechnol 20: 87-90.
Nair M P, Schwartz S A, Mahajan S D, Tsiao C, Chawda R P, Whitney R, Don Sykes B B, Hewitt R. 2004. Drug abuse and neuropathogenesis of HIV infection: role of DC-SIGN and IDO. J Neuroimmunol 157: 56-60.
Newman T A, Galea I, van Rooijen N, Perry V H. 2005. Blood-derived dendritic cells in an acute brain injury. J Neuroimmunol 166: 167-172.
Pachter J S, de Vries H E, Fabry Z. 2003. The blood-brain barrier and its role in immune privilege in the central nervous system. J Neuropathol Exp Neurol 62: 593-604.
Parent J M, Lowenstein D H. 2002. Seizure-induced neurogenesis: are more new neurons good for an adult brain? Prog Brain Res 135: 121-31.
Pashenkov M, Teleshova N, Link H. 2003. Inflammation in the central nervous system: the role for dendritic cells. Brain Pathol 13: 23-33.
Paxinos G, Franklin K B J. 2001. The mouse brain in sterotaxic coordinates. San Diego: Academic Press.
Peretto P, Merighi A, Fasolo A, Bonfanti L. 1999. The subependymal layer in rodents: a site of structural plasticity and cell migration in the adult mammalian brain. Brain Res Bull 49: 221-243.
Perry V H, Hume D A, Gordon S. 1985. Immunohistochemical localization of macrophages and microglia in the adult and developing mouse brain. Neuroscience 15: 313-326.
Peters A, Palay S L, Webster HdeF. 1991. The fine structure of the nervous system, 3rd ed. New York: Oxford University Press.
Pile-Spellman J M, McKusick K A, Strauss H W, Cooney J, Taveras J M. 1984. Experimental in vivo imaging of the cranial perineural lymphatic pathway. AJNR Am J Neuroradiol 5: 539-545.
Plesnila N, Zinkel S, Le D A, Amin-Hanjani S, Wu Y, Qiu J, Chiarugi A, Thomas S S, Kohane D S, Korsmeyer S J, Moskowitz M A (2001) BID mediates neuronal cell death after oxygen/glucose deprivation and focal cerebral ischemia. Proc Natl Acad Sci USA 98:15318-15323.
Raivich G, Bohatschek M, Kloss C U, Werner A, Jones L L, Kreutzberg G W. 1999. Neuroglial activation repertoire in the injured brain: graded response, molecular mechanisms and cues to physiological function. Brain Res Rev 30: 77-105.
Ramirez-Castillejo C, Nacher J, Molowny A, Ponsoda X, Lopez-Garcia C. 2002. PSA-NCAM immunocytochemistry in the cerebral cortex and other telencephalic areas of the lizard Podarcis hispanica: differential expression during medial cortex neuronal regeneration. J Comp Neurol 453: 145-156.
Rasband W S. 1997-2004. Image J. Bethesda, Md.: National Institutes of Health.
Reichmann G, Schroeter M, Jander S, Fischer H G. 2002. Dendritic cells and dendritic-like microglia in focal cortical ischemia of the mouse brain. J Neuroimmunol 129: 125-132.
Rosen H, Gordon S. 1987. Monoclonal antibody to the murine type 3 complement receptor inhibits adhesion of myelomonocytic cells in vitro and the inflammatory cell recruitment in vivo. J Exp Med 166: 1685-1701.
Rosicarelli B, Serafini B, Sbriccoli M, Lu M, Cardone F, Pocchiari M, Aloisi F. 2005. Migration of dendritic cells into the brain in a mouse model of prion disease. J Neuroimmunol 165: 114-120.
Sanchez-Madrid F, Thompson S S, Springer T A. 1983. Mapping of antigenic and functional epitopes on the alpha- and beta-subunits of two related mouse glycoproteins involved in cell interactions, LFA-1 and Mac 1. J Exp Med 158: 586-602.
Santambrogio L, Belyanskaya S L, Fischer F R, Cipriani B, Brosnan C F, Ricciardi-Castagnoli P, Stern L J, Strominger J L, Riese R. 2001. Developmental plasticity of CNS microglia. Proc Natl Acad Sci USA 98: 6295-6300.
Sasmono R T, Oceandy D, Pollard J W, Tong W, Pavli P, Wainwright B J, Ostrowski M C, Himes S R, Hume D A. 2003. A macrophage colony-stimulating factor receptor-green fluorescent protein transgene is expressed throughout the mononuclear phagocyte system of the mouse. Blood 101: 1155-1163.
Schwartz M, Yoles E. 2006. Immune-based therapy for spinal cord repair: autologous macrophages and beyond. J Neurotrauma 23: 360-370.
Schwartz M, Butovsky O, Bruck W, Hanisch U K. 2006. Microglia phenotype: is the commitment reversible? Trends Neurosci 29: 68-74.
Serafini B, Rosicarelli B, Magliozzi R, Stigliano E, Capello E, Mancardi G L, Aloisi F. 2006. Dendritic cells in multiple sclerosis lesions: maturation stage, myelin uptake, and interaction with proliferating T cells. J Neuropathol Exp Neurol 65: 124-141.
Sidman R L. 1971. Atlas of the mouse brain and spinal cord. Cambridge, Mass.: Harvard University Press.
Sierra A, Gottfried-Blackmore A C, McEwen B S, Bulloch K. 2007. Microglia derived from aging mice exhibit an altered inflammatory profile. Glia 55: 412-424.
Stallcup W B, Dahlin K, Healy P. 1990. Interaction of the NG2 chondroitin sulfate proteoglycan with type VI collagen. J Cell Bio 111: 3177-3188.
Steinman R M. 2003. Some interfaces of dendritic cell biology. APMIS 111: 675-697.
Steinman R, Inaba K. 1989. Immunogenicity: role of dendritic cells. Bioessays 10: 145-152.
Steinman R M, Witmer M D, Nussenzweig M C, Chen L L, Schlesinger S, Cohn Z A. 1980. Dendritic cells of the mouse: identification and characterization. J Invest Dermatol 75: 14-16.
Stichel C C, Luebbert H. 2006. Inflammatory processes in the aging mouse brain: participation of dendritic cells and T-cells. Neurobiol Aging (in press).
Suter T, Biollaz G, Gatto D, Bernasconi L, Herren T, Reith W, Fontana A. 2003. The brain as an immune privileged site: dendritic cells of the central nervous system inhibit T cell activation. Eur J Immunol 33: 2998-3006.
Thorne R G, Pronk G J, Padmanabhan V, Frey W H 2nd. 2004. Delivery of insulin-like growth factor-I to the rat brain and spinal cord along olfactory and trigeminal pathways following intranasal administration. Neuroscience 127: 481-496.
Trifilo M J, Lane T E. 2004. The CC chemokine ligand 3 regulates CD11c+CD11b+CD8alpha-dendritic cell maturation and activation following viral infection of the central nervous system: implications for a role in T cell activation. Virology 327: 8-15.
Unal-Cevik I, Kilinç M, Gürsoy-Ozdemir Y, Gurer G, Dalkara T. 2004. Loss of NeuN immunoreactivity after cerebral ischemia does not indicate neuronal cell loss: a cautionary note. Brain Res 1015: 169-174.
Walter B A, Valera V A, Takahashi S, Ushiki T. 2006. The olfactory route for cerebrospinal fluid drainage into the peripheral lymphatic system. Neuropathol Appl Neurobiol 32: 388-396.
Yue X, Favot P, Dunn T L, Cassady A I, Hume D A. 1993. Expression of mRNA encoding the macrophage colony-stimulating factor receptor (c-fms) is controlled by a constitutive promoter and tissuespecific transcription elongation. Mol Cell Biol. 1993; 13: 3191-3201.
Ziv Y, Avidan H, Pluchino S, Martino G, Schwartz M. 2006a. Synergy between immune cells and adult neural stem/progenitor cells promotes functional recovery from spinal cord injury. Proc Natl Acad Sci USA 103: 13174-13179.
Ziv Y, Ron N, Butovsky O, Landa G, Sudai E, Greenberg N, Cohen H, Kipnis J, Schwartz M. 2006b. Immune cells contribute to the maintenance of neurogenesis and spatial learning abilities in adulthood. Nat Neurosci 9: 268-275.

We claim:

1. An isolated brain dendritic cell from a mammal, wherein the mammal is not suffering from an inflammatory disease or condition.

2. The isolated brain dendritic cell of claim 1, which cell is characterized by a marker phenotype selected from the group consisting of CD11c+, CD11b+, Iba-1+, CD45+, F4/80+, NeuN−, DCX−, NG2−, GFAP−, and combinations thereof.

3. The isolated brain dendritic cell of claim 2, which cell is characterized by a marker expression phenotype that is {CD11c+, CD11b+, Iba-1+, CD45+, F4/80+, NeuN−, DCX−, NG2−, and GFAP−}.

4. A method of identifying brain dendritic cells comprising steps of:
(a) providing a transgenic animal that expresses a detectable agent under the control of the CD11c promoter;
(b) detecting the agent in brain cells or brain tissue;
(c) determining, based on the presence of the agent in a given cell, that the cell is a brain dendritic cell.

5. The method of claim 4, wherein the detectable agent is a fluorescent protein.

6. The method of claim 5, wherein the detectable agent is yellow fluorescent protein.

7. The method of claim 4, further comprising determining expression of a marker selected from the group consisting of CD11b, Iba-1, CD45, F4/80, and combinations thereof, wherein expression of the marker is an indicator that the cell is a brain dendritic cell.

8. The method of claim 4, further comprising determining expression of a marker selected from the group consisting of NeuN, NG2 proteoglycan, GFAP, and combinations thereof, wherein expression of the marker is an indicator that the cell is not a brain dendritic cell.

9. The method of any one of claims 4-8, further comprising a step of determining expression of one or more additional markers expressed by the cells.

10. The method of claim 9, further comprising a step of determining that one or more of the additional markers are not expressed by other brain cells.

11. A method for identifying brain dendritic cells in a brain tissue sample comprising steps of:
(a) providing a map that indicates the distribution of brain dendritic cells in the brain of an animal at a given age;
(b) providing an image of the brain tissue sample from a test animal, wherein the tissue sample comprises at least one cell suspected of being a brain dendritic cells;
(c) comparing the image of the brain tissue sample with the map, wherein the map indicates distribution of brain dendritic cells at an age comparable to that of the age of the test animal; and
(d) identifying, based on the comparison, that the at least one cell is a brain dendritic cell.

12. The method of claim 11, wherein the map is part of a series of maps comprising an atlas, wherein maps in the series represent distribution maps of brain dendritic cells at various ages.

13. The method of claim 11, wherein the tissue sample has been processed to detect a a marker selected from the group consisting of CD11c, CD11b, Iba-1, CD45, F4/80, and combinations thereof.

14. A method for isolating brain dendritic cells, comprising steps of:
(a) identifying brain dendritic cells according to the method of claim 11; and
(b) isolating the identified brain dendritic cells from the sample.

15. A method of isolating one or more brain dendritic cells, the method comprising steps of:
(a) providing a transgenic mammal that expresses a detectable agent under the control the CD11c promoter;
(b) detecting the agent in brain cells or brain tissue; and
(c) isolating cells that express the agent from cells that do not express the agent.

16. The method of claims 15, wherein the detectable agent is a fluorescent molecule.

17. The method of claim 16, wherein the detectable agent is yellow fluorescent protein.

18. The method of claim 16, wherein the step of isolating comprises performed fluorescence-activated cell sorting.

19. The method of claims 15, wherein the step of isolating comprises performing laser capture microdissection.

20. A method of isolating one or more brain dendritic cells, the method comprising steps of methods of isolating generally comprise steps of:
(a) providing a starting population of cells derived from brain tissue; and
(b) sorting the population of cells into subpopulations based on characteristics of brain dendritic cells, wherein at least one of the subpopulations comprises a substantially higher proportion of brain dendritic cells than that of the starting population.

21. A method of identifying genes that are differentially expressed in brain dendritic cells comprising steps of:
(a) obtaining RNA from brain dendritic cells; and
(b) detecting or identifying one or more genes that are differentially expressed in brain dendritic cells as compared to a control sample.

22. The method of claim 21, wherein the step of isolating is performed during a particular period of development.

23. The method of claim 21, wherein the step of isolating is performed during a period during which a particular developmental process is known to take place.

24. The method of claim 23, wherein the developmental process is selected from the group consisting of neurogenesis, gliogenesis, synaptogenesis, embryogenesis, and apoptosis.

25. The method of claim 24, wherein the brain dendritic cells are isolated from an animal with a condition selected from the group consisting of ischemic injury, excitotoxic injury, autoimmune disorders, and combinations thereof.

26. A method for detecting or identifying genes involved in a neurological disease comprising a step of detecting or identifying one or more genes that are differentially regulated in an animal that is a model for a neurodegenerative disease and that contains detectably labeled brain dendritic cells as compared to a control sample, wherein the control sample comprises RNA obtained from cells from an animal that is not a model for the neurological disease.

27. The method of claim 26, wherein the transgenic animal bears a transgene for a detectable agent under the control of the CD11c promoter.

28. The method of claim 27, wherein the detectable agent is a fluorescent molecule.

29. The method of claim 28, wherein the detectable agent is yellow fluorescent protein.

30. The method of claim 29, wherein the neurological disease is selected from the group consisting of Alzheimer's Disease, Parkinson's Disease, Huntington's Disease, multiple sclerosis, and combinations thereof.

31. A method of identifying agents that modulate brain dendritic cells, the method comprising steps of:
(a) providing a sample that contains brain dendritic cells;
(b) contacting the sample with a test agent;
(c) determining whether the test agent modulates one or more aspects of brain dendritic cell development, activity, gene expression, and localization; and
(d) identifying, based on the determination, that the test agent as a modulator of brain dendritic cells.

32. The method of claim 31, wherein the aspect comprises an activity selected from the group consisting of expression of MHC (major histocompatibility complex) molecules, cytokines, cytokine receptors, and combinations thereof.

33. The method of claim 32, wherein the expression is induced by a cytokine.

34. The method of claim 33, wherein the cytokine is interferon gamma.

35. The method of claim 34, wherein the activity comprises expression of MHC class II molecules.

36. The method of claim 32, wherein the activity comprises expression of a cytokine selected from the group consisting of: TNFα, IL-6, nitric oxide, and combinations thereof.

37. The method of claim 36, wherein the aspect comprises expression of a gene selected from the group consisting of resistin-like alpha, CCL17, CxCL9, CD209a (DC-Sign), H2-Eb1, Spp1 (Osteopontin), Axl, H2-Aa, H2-Al, CxCl2 (MIP-2), Clec7a (Dectin-1), CCR2, Itgax (CD11c), IGF-1, CD36, and combinations thereof.
